(19) United States
(12) Patent Application Publication
(10) Pub. No.: US 2012/0324604 A1
(43) Pub. Date: Dec. 20, 2012

(54) PESTICIDAL MIXTURES CONTAINING ISOXAZOLINE DERIVATIVES AND A FUNGICIDE

(75) Inventors: Ana Cristina Dutton, Stein (CH); Jerome Yves Cassayre, Stein (CH); Ulrich Johannes Haas, Stein (CH); Andrew John Leadbeater, Stein (CH)

(73) Assignee: SYNGENTA CROP PROTECTION LLC, Greensboro, NC (US)

(21) Appl. No.: 13/581,177

(22) PCT Filed: Feb. 3, 2011
(86) PCT No.: PCT/EP2011/051512

§ 371 Date: Aug. 24, 2012

(30) Foreign Application Priority Data
Feb. 25, 2010 (EP) 10250336.4
May 7, 2010 (GB) 1007689.1
May 28, 2010 (EP) 10164234.6
Oct. 12, 2010 (EP) 10187269.5

(51) Int. Cl. A01N 43/80; A01P 7/04; A01H 5/10; A01P 5/00; A01P 9/00; A01N 43/54; A01P 7/02

(52) U.S. Cl. 800/298; 514/269; 514/378

(57) ABSTRACT

The present invention relates to pesticidal mixtures comprising a component A and a component B, wherein component A is a compound of formula (I) wherein A¹, A², R¹, R², R³, R⁴ and R⁵ are as defined in claim 1, and one of Y¹ and Y² is S, SO or SO₂ and the other is CH₂ and component B is a fungicide as defined in claim 1, The present invention also relates to methods of using said mixtures for the control of plant pests.

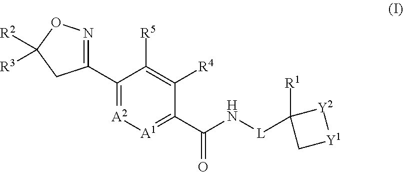

The present invention relates to mixtures of pesticidally active ingredients and to methods of using the mixtures in the field of agriculture.

WO 2009/080250 discloses that certain isoxazoline compounds have insecticidal activity.

The present invention provides pesticidal mixtures comprising a component A and a component B, wherein component A is a compound of formula I

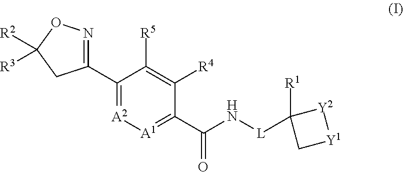

wherein
one of Y¹ and Y² is S, SO or SO₂ and the other is CH₂;
L is a direct bond or methylene;
A¹ and A² are C—H, or one of A¹ and A² is C—H and the other is N;
R¹ is hydrogen or methyl;
R² is chlorodifluoromethyl or trifluoromethyl;
R³ is 3,5-dibromo-phenyl, 3,5-dichloro-phenyl, 3,4-dichloro-phenyl, or 3,4,5-trichloro-phenyl;
R⁴ is methyl;
R⁵ is hydrogen;
or R⁴ and R⁵ together form a bridging 1,3-butadiene group;
and component B is a fungicide selected from
a strobilurin fungicide including those selected from the group consisting of: Azoxystrobin, Dimoxystrobin, Enestrobin, Fluoxastrobin, Kresoxim-methyl, Metominostrobin, Orysastrobin, Picoxystrobin, Pyraclostrobin and Trifloxystrobin;
an azole fungicide including those selected from the group consisting of: Azaconazole, Bromuconazole, Cyproconazole, Difenoconazole, Diniconazole, Diniconazole-M, Epoxiconazole, Fenbuconazole, Fluquinconazole, Flusilazole, Flutriafol, Hexaconazole, Imazalil, Imibenconazole, Ipconazole, Metconazole, Myclobutanil, Oxpoconazole, Pefurazoate, Penconazole, Prochloraz, Propiconazole, Prothioconazole, Simeconazole, Tebuconazole, Tetraconazole, Triadimefon, Triadimenol, Triflumizole, Triticonazole, Diclobutrazol, Etaconazole, Furconazole, Furconazole-cis, Thiabendazole and Quinconazole;
a phenyl pyrrole fungicide including those selected from the group consisting of: Fenpiclonil and Fludioxonil;
an anilino-pyrimidine fungicide including those selected from the group consisting of: Cyprodinil, Mepanipyrim and Pyrimethanil;
a morpholine fungicide including those selected from the group consisting of: Aldimorph, Dodemorph, Fenpropimorph, Tridemorph, Fenpropidin and Spiroxamine;
a carboxamide fungicide including those selected from the group consisting of: Isopyrazam, Sedaxane, Bixafen, Penthiopyrad, Fluxapyroxad, Boscalid, Penflufen, Fluopyram, a compound of formula II

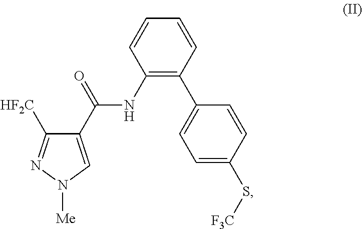

a compound of formula III

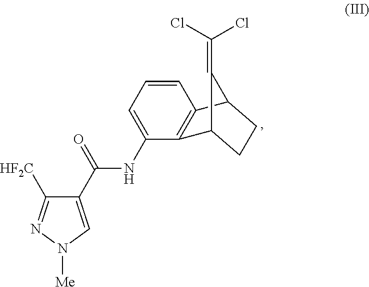

and a compound of formula IV

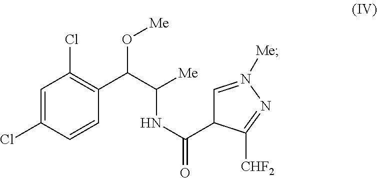

a carboxylic acid amide fungicide including those selected from the group consisting of: Mandipropamid, Benthiavalicarb and Dimethomorph;
Chlorothalonil, Fluazinam, Dithianon, Metrafenone, Tricyclazole, Mefenoxam, Metalaxyl, Acibenzolar, Mancozeb, Ametoctradine and Cyflufenamid.

Compounds of formula I are known to have insecticidal activity, whereas compounds of component B are known to have fungicidal activity. Certain active ingredient mixtures of a compound of formula I and a fungicide can enhance the spectrum of action with respect to the pest to be controlled, e.g. the animal pest and/or the fungal pest. For example, the combination of A and B may cause an increase in the insecticidal action of component A and/or an increase in the fungicidal action of component B which would be expected from each component when used alone. This allows, on the one hand, a substantial broadening of the spectrum of pests that can be controlled and, on the other hand, increased safety in use through lower rates of application.

However, besides the actual synergistic action with respect to pest control, the pesticidal mixtures according to the invention can have further advantageous properties which can also be described, in a wider sense, as synergistic activity. Examples of such advantageous properties that may be mentioned are: a broadening of the spectrum of activity; a reduction in the rate of application of the active ingredients; adequate pest control with the aid of the mixtures according to the invention, sometimes even at a rate of application at which the individual compounds are totally ineffective; advantageous behaviour during formulation and/or upon application, for example upon grinding, sieving, emulsifying, dissolving or dispensing; increased storage stability; improved stability to light; more advantageous degradability; improved toxicological and/or ecotoxicological behaviour; improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigor, and early germination; or any other advantages familiar to a person skilled in the art.

The compounds of formula I and their manufacturing processes are known from WO 2009/080250. The components B are known and have the following CAS numbers: Azoxystrobin (131860-33-8), Dimoxystrobin (149961-52-4), Enestrobin (238410-11-2), Fluoxastrobin (193740-76-0), Kresoxim-methyl (143390-89-0), Metominostrobin (133408-50-1), Orysastrobin (248593-16-0), Picoxystrobin (117428-22-5), Pyraclostrobin (175013-18-0), trifloxystrobin (141517-21-7), Azaconazole (60207-31-0), Bromuconazole (116255-48-2), Cyproconazole (94361-06-5), Difenoconazole (119446-68-3), Diniconazole (83657-24-3), Diniconazole-M (83657-18-5), Epoxiconazole (13385-98-8), Fenbuconazole (114369-43-6), Fluquinconazole (136426-54-5), Flusilazole (85509-19-9), Flutriafol (76674-21-0), Hexaconazole (79983-71-4), Imazalil (58594-72-2), Imibenconazole (86598-92-7), Ipconazole (125225-28-7), Metconazole (125116-23-6), Myclobutanil (88671-89-0), Oxpoconazole (174212-12-5), Pefurazoate (58011-68-0), Penconazole (66246-88-6), Prochloraz (67747-09-5), Propiconazole (60207-90-1), Prothioconazole (178928-70-6), Simeconazole (149508-90-7), Tebuconazole (107534-96-3), Tetraconazole (112281-77-3), Triadimefon (43121-43-3), Triadimenol (55219-65-3), Triflumizole (99387-89-0), Triticonazole (131983-72-7), Diclobutrazol (76738-62-0), Etaconazole (60207-93-4), Fluconazole (86386-73-4), Fluconazole-cis (112839-32-4), Thiabendazole (148-79-8), Quinconazole (103970-75-8), Fenpiclonil (74738-17-3), Fludioxonil (131341-86-1), Cyprodinil (121552-61-2), Mepanipyrim (110235-47-7), Pyrimethanil (53112-28-0), Aldimorph (91315-15-0), Dodemorph (1593-77-7), Fenpropimorph (67564-91-4), Tridemorph (81412-43-3), Fenpropidin (67306-00-7), Spiroxamine (118134-30-8), Isopyrazam (881685-58-1), Sedaxane (874967-67-6), Bixafen (581809-46-3), Penthiopyrad (183675-82-3), Fluxapyroxad (907204-31-3), Boscalid (188425-85-6), Penflufen (494793-67-8), Fluopyram (658066-35-4), Mandipropamid (374726-62-2), Benthiavalicarb (413615-35-7), Dimethomorph (110488-70-5), Chlorothalonil (1897-45-6), Fluazinam (79622-59-6), Dithianon (3347-22-6), Metrafenone (220899-03-6), Tricyclazole (41814-78-2), Mefenoxam (70630-17-0), Metalaxyl (57837-19-1), Acibenzolar (126448-41-7) (Acibenzolar-5-methyl (126448-41-7)), Mancozeb (8018-01-7), Ametoctradine (865318-97-4) Cyflufenamid (180409-60-3), and Kresoxim-methyl (143390-89-0). The compound of formula II is known from WO 2008/053044, the compound of formula III is known from WO 2007/048556, the compound of formula IV is known from WO 2010/000612.

Reference to the above components B includes reference to their salts and any usual derivatives, such as ester derivatives. In particular, reference to Acibenzolar includes reference to, and is preferably, Acibenzolar-S-methyl.

The combinations according to the invention may also comprise more than one of the active components B, if, for example, a broadening of the spectrum of pest control is desired. For instance, it may be advantageous in the agricultural practice to combine two or three components B with any of the compounds of formula I, or with any preferred member of the group of compounds of formula I. The mixtures of the invention may also comprise other active ingredients in addition to components A and B. In other embodiments the mixtures of the invention may include only components A and B as pesticidally active ingredients, e.g. no more than two pesticidally active ingredients.
In one preferred group of compounds of formula I Y¹ is S and Y² is CH₂.
In another preferred group of compounds of formula I Y¹ is SO and Y² is CH₂.
In another preferred group of compounds of formula I Y¹ is SO₂ and Y² is CH₂.
In another preferred group of compounds of formula I Y² is S and Y¹ is CH₂.
In another preferred group of compounds of formula I Y² is SO and Y¹ is CH₂.
In another preferred group of compounds of formula I Y² is SO₂ and Y¹ is CH₂.
In yet another preferred group of compounds of formula I L is a direct bond or methylene; one of Y¹ and Y² is S and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond or methylene; one of Y¹ and Y² is SO and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond or methylene; one of Y¹ and Y² is SO₂ and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond or methylene; one of Y¹ and Y² is S and the other is CH₂; A¹ and A² are C—H; R¹ is hydrogen or methyl; R² is trifluoromethyl; R³ is 3,5-dichloro-phenyl; and R⁴ is methyl; and R⁴ and R⁵ together form a bridging 1,3-butadiene group.
In yet another preferred group of compounds of formula I L is a direct bond or methylene; one of Y¹ and Y² is SO and the other is CH₂; A¹ and A² are C—H; R¹ is hydrogen or methyl; R² is trifluoromethyl; R³ is 3,5-dichloro-phenyl; and R⁴ is methyl; and R⁴ and R⁵ together form a bridging 1,3-butadiene group.
In yet another preferred group of compounds of formula I L is a direct bond or methylene; one of Y¹ and Y² is SO₂ and the other is CH₂; A¹ and A² are C—H; R¹ is hydrogen or methyl; R² is trifluoromethyl; R³ is 3,5-dichloro-phenyl; and R⁴ is methyl; and R⁴ and R⁵ together form a bridging 1,3-butadiene group.
In yet another preferred group of compounds of formula I L is a direct bond or methylene; one of Y¹ and Y² is S and the other is CH₂; A¹ is C—H; A² is N; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond or methylene; one of Y¹ and Y² is SO and the other is CH₂; A¹ is C—H; A² is N; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond or methylene; one of Y¹ and Y² is SO₂ and the other is CH₂; A¹ is C—H; A² is N; R¹ is hydrogen or methyl; R² is trifluoromethyl; R³ is 3,5-dichloro-phenyl; R⁴ is methyl; and R⁵ is hydrogen.
In yet another preferred group of compounds of formula I L is a direct bond; Y¹ is S, SO or SO₂; Y² is CH₂; A¹ is C—H; A² is C—H; R¹ is hydrogen; R² is trifluoromethyl; R³ is 3,5-dichloro-phenyl; R⁴ is methyl; and R⁵ is hydrogen.
In yet another preferred group of compounds of formula I L is a direct bond; Y¹ is S, SO or SO₂; Y² is CH₂; A¹ is C—H; A² is C—H; R¹ is methyl; R² is trifluoromethyl; R³ is 3,5-dichloro-phenyl; R⁴ is methyl; and R⁵ is hydrogen.
In yet another preferred group of compounds of formula I L is methylene; Y¹ is CH₂; Y² is S, SO or SO₂; A¹ is C—H; A² is C—H; R¹ is hydrogen; R² is trifluoromethyl; R³ is 3,5-dichloro-phenyl; R⁴ is methyl; and R⁵ is hydrogen.
In yet another preferred group of compounds of formula I L is methylene; Y¹ is CH₂; Y² is S, SO or SO₂; A¹ is C—H; A² is C—H; R¹ is methyl; R² is trifluoromethyl; R³ is 3,5-dichloro-phenyl; R⁴ is methyl; and R⁵ is hydrogen
Preferably when L is a direct bond Y² is CH₂ and Y¹ is S, SO or SO₂ and when L is methylene Y² is S, SO or SO₂ and Y¹ is CH₂.

Each substituent definition in each alternative preferred groups of compounds of formula I may be juxtaposed with any substituent definition in any other preferred group of compounds, in any combination.

Compounds of formula I include at least one chiral centre and may exist as compounds of formula I* or compounds of formula I**.

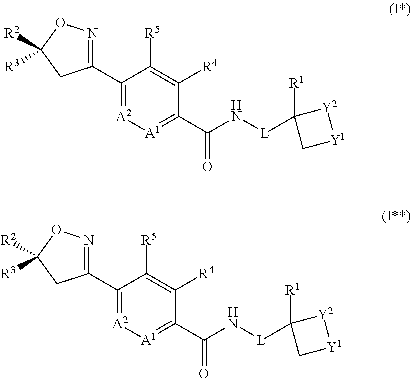

Compounds of formula I** are more biologically active than compounds of formula I* (confirmed by X-ray analysis). Component A may be a mixture of compounds I* and I** in any ratio e.g. in a molar ratio of 1:99 to 99:1, e.g. 10:1 to 1:10, e.g. a substantially 50:50 molar ratio. Preferably component A is a racemic mixture of the compounds of formula I** and I* or is enantiomerically enriched for the compound of formula I**. For example, when component A is an enantiomerically enriched mixture of formula I**, the molar proportion of compound I** compared to the total amount of both enantiomers is for example greater than 50%, e.g. at least 55, 60, 65, 70, 75, 80, 85, 90, 95, 96, 97, 98, or at least 99%.

Preferred compounds of formula I are shown in the Table below.

TABLE A
Compounds of formula (Ia)
(Ia)

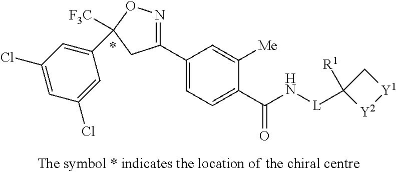

| Comp | Stereochemistry | | | | |
|---|---|---|---|---|---|
| No. | at * | L | R¹ | Y¹ | Y² |
| 1 | racemic mixture | bond | CH₃ | S | CH₂ |
| 2 | racemic mixture | bond | CH₃ | SO (cis) | CH₂ |
| 3 | racemic mixture | bond | CH₃ | SO (trans) | CH₂ |
| 4 | racemic mixture | bond | CH₃ | SO₂ | CH₂ |
| 5 | racemic mixture | bond | H | S | CH₂ |
| 6 | racemic mixture | bond | H | SO (cis) | CH₂ |
| 7 | racemic mixture | bond | H | SO (trans) | CH₂ |
| 8 | racemic mixture | bond | H | SO₂ | CH₂ |
| 9 | racemic mixture | CH₂ | CH₃ | CH₂ | S |
| 10 | racemic mixture | CH₂ | CH₃ | CH₂ | SO (cis) |
| 11 | racemic mixture | CH₂ | CH₃ | CH₂ | SO (trans) |
| 12 | racemic mixture | CH₂ | CH₃ | CH₂ | SO₂ |
| 13 | racemic mixture | CH₂ | H | CH₂ | S |
| 14 | racemic mixture | CH₂ | H | CH₂ | SO (cis) |
| 15 | racemic mixture | CH₂ | H | CH₂ | SO (trans) |
| 16 | racemic mixture | CH₂ | H | CH₂ | SO₂ |
| 19 | S | bond | CH₃ | S | CH₂ |
| 20 | S | bond | CH₃ | SO (cis) | CH₂ |
| 21 | S | bond | CH₃ | SO (trans) | CH₂ |
| 22 | S | bond | CH₃ | SO₂ | CH₂ |
| 23 | S | bond | H | S | CH₂ |
| 24 | S | bond | H | SO (cis) | CH₂ |
| 25 | S | bond | H | SO (trans) | CH₂ |
| 26 | S | bond | H | SO₂ | CH₂ |
| 27 | S | CH₂ | CH₃ | CH₂ | S |
| 28 | S | CH₂ | CH₃ | CH₂ | SO (cis) |
| 29 | S | CH₂ | CH₃ | CH₂ | SO (trans) |
| 30 | S | CH₂ | CH₃ | CH₂ | SO₂ |
| 31 | S | CH₂ | H | CH₂ | S |
| 32 | S | CH₂ | H | CH₂ | SO (cis) |
| 33 | S | CH₂ | H | CH₂ | SO (trans) |
| 34 | S | CH₂ | H | CH₂ | SO₂ |

The present invention includes all isomers of compounds of formula (I), salts and N-oxides thereof, including enantiomers, diastereomers and tautomers. Component A may be a mixture of any type of isomer of a compound of formula I, or may be substantially a single type of isomer. For example, where Y¹ or Y² is SO, component A may be a mixture of the cis and trans isomer in any ratio, e.g. in a molar ratio of 1:99 to 99:1, e.g. 10:1 to 1:10, e.g. a substantially 50:50 molar ratio. For example, in trans enriched mixtures of the compound of formula I, e.g. when Y¹ or Y² is SO, the molar proportion of the trans compound in the mixture compared to the total amount of both cis and trans is for example greater than 50%, e.g. at least 55, 60, 65, 70, 75, 80, 85, 90, 95, 96, 97, 98, or at least 99%. Likewise, in cis enriched mixtures of the compound of formula I (preferred), e.g. when Y¹ or Y² is SO, the molar proportion of the cis compound in the mixture compared to the total amount of both cis and trans is for example greater than 50%, e.g. at least 55, 60, 65, 70, 75, 80, 85, 90, 95, 96, 97, 98, or at least 99%. The compound of formula I may be enriched for the trans sulphoxide. Likewise, the compound of formula I may be enriched for the cis sulphoxide. Y¹ or Y² is SO for compounds 2, 3, 6, 7, 10, 11, 14, 15, 20, 21, 24, 25, 28, 29, 32 and 33 in Table A. Each may be a mixture which is enriched for the cis or trans isomer respectively.

In one embodiment of the invention component B is a compound selected from the group consisting of Azoxystrobin, Isopyrazam, Chlorothalonil, Cyproconazole, Difenoconaozle, Mandipropamid, Mefenoxam, Metalaxyl, Sedaxane, Acibenzolar (including Acibenzolar-S-methyl), Fludioxonil, Cyprodinil, Penconazole, Propiconazole, Mancozeb, Prothioconazole, Pyraclostrobin, Boscalid, Bixafen, Fluopyram, Penthiopyrad, Thiabendazole, Fluazinam, Fenpropidin, Cyclufenamid, Tebuconaozle, Trifoxystrobin, Fluxapyroxad, Penflufen, Fluoxastrobin, Kresoxim-methyl, Benthiavalicarb, Dimethomorph, a compound of formula II, a compound of formula III and a compound of formula IV.

Preferably component B is a compound selected from the group consisting of Azoxystrobin, Isopyrazam, Chlorothalonil, Cyroconazole, Difenoconaozle, Mandipropamid, Mefenoxam, Metalaxyl, Sedaxane, Acibenzolar (including Acibenzolar-S-methyl), Fludioxonil, Cyprodinil, Penconazole, Propiconazole, Mancozeb, Prothioconazole, Pyraclostrobin, Boscalid, Bixafen, Fluopyram, Penthiopyrad, Thiabendazole, a compound of formula III and a compound of formula IV.

More preferably component B is a compound selected from the group consisting of Azoxystrobin, Isopyrazam, Chlorothalonil, Cyroconazole, Difenoconaozle, Mandipropamid, Mefenoxam, Metalaxyl, Sedaxane, Acibenzolar (including Acibenzolar-S-methyl), Fludioxonil, Thiabendazole, a compound of formula III and a compound of formula IV. More preferably component B is a compound selected from the group consisting of Azoxystrobin, Isopyrazam, Chlorothalonil, Cyproconazole, Difenoconaozle, Mandipropamid, Mefenoxam, a compound of formula III and a compound of formula IV.

The invention also relates to the following combinations:

A mixture of a compound from Table A and Azoxystrobin.

A mixture of a compound from Table A and Isopyrazam.

A mixture of a compound from Table A and Chlorothalonil.

A mixture of a compound from Table A and Cyproconazole.

A mixture of a compound from Table A and Difenoconaozle.

A mixture of a compound from Table A and Mandipropamid.

A mixture of a compound from Table A and Mefenoxam.

A mixture of a compound from Table A and Metalaxyl.

A mixture of a compound from Table A and Sedaxane.

A mixture of a compound from Table A and Acibenzolar.

A mixture of a compound from Table A and Acibenzolar-S-Methyl.

A mixture of a compound from Table A and a compound of formula II.

A mixture of a compound from Table A and a compound of formula III.

A mixture of a compound from Table A and a compound of formula IV.

A mixture of a compound from Table A and Fludioxonil.

A mixture of a compound from Table A and Cyprodinil.

A mixture of a compound from Table A and Penconazole.

A mixture of a compound from Table A and Propiconazole.

A mixture of a compound from Table A and Mancozeb.

A mixture of a compound from Table A and Prothioconazole.

A mixture of a compound from Table A and Pyraclostrobin.

A mixture of a compound from Table A and Boscalid.

A mixture of a compound from Table A and Bixafen.

A mixture of a compound from Table A and Fluopyram.

A mixture of a compound from Table A and Penthiopyrad.

A mixture of a compound from Table A and Thiabendazole.

A mixture of a compound from Table A and Fluazinam.

A mixture of a compound from Table A and Fenpropidin.

A mixture of a compound from Table A and Cyclufenamid.

A mixture of a compound from Table A and Tebuconaozle.

A mixture of a compound from Table A and Trifoxystrobin.

A mixture of a compound from Table A and Fluoxastrobin.

A mixture of a compound from Table A and Kresoxim-methyl.

A mixture of a compound from Table A and Benthiavalicarb.

A mixture of a compound from Table A and Dimethomorph.

A mixture of a compound from Table A and Ametoctradine.

A mixture of a compound from Table A and Fluxapyroxad.

A mixture of a compound from Table A and Penflufen.

The present invention also relates to a method of controlling phytopathogenic diseases on useful plants or on propagation material thereof, which comprises applying to the useful plants, the locus thereof or propagation material thereof a combination of components A and B; a method of controlling insects, acarines, nematodes or molluscs which comprises applying to a pest, to a locus of a pest, or to a plant susceptible to attack by a pest a combination of components A and B; a seed comprising a pesticidal mixture of components A and B; a method comprising coating a seed with a mixture of components A and B.

Components A and B may be provided and/or used in amounts such that they are capable of synergistic pest control. For example, the present invention includes pesticidal mixtures comprising a component A and a component B in a synergistically effective amount; agricultural compositions comprising a mixture of component A and B in a synergistically effective amount; the use of a mixture of component A and B in a synergistically effective amount for combating animal pests; the use of a mixture of component A and B in a synergistically effective amount for combating phytopathogenic fungi; a method of combating animal pests which comprises contacting the animal pests, their habit, breeding ground, food supply, plant, seed, soil, area, material or environment in which the animal pests are growing or may grow, or the materials, plants, seeds, soils, surfaces or spaces to be protected from animal attack or infestation with a mixture of component A and B in a synergistically effective amount; a method for protecting crops from attack or infestation by animal pests and/or phythopathogenic fungi, which comprises contacting a crop with a mixture of component A and B in a synergistically effective amount; a method for the protection of seeds from soil insects and of the seedlings' roots and shoots from soil and foliar insects and/or phythopathogenic fungi comprising contacting the seeds before sowing and/or after pre-germination with a mixture of component A and B in a synergistically effective amount; seeds comprising, e.g. coated with, a mixture of component A and B in a synergistically effective amount; a method comprising coating a seed with a mixture of component A and B in a synergistically effective amount; a method of controlling phytopathogenic, e.g. fungal, diseases on useful plants or on propagation material thereof, which comprises applying to the useful plants, the locus thereof or propagation material thereof a combination of components A and B in a synergistically effective amount. In such applications the mixtures of A and B will normally be applied in a fungicidally effective amount. The invention also provides a method of controlling insects, acarines, nematodes or molluscs which comprises applying to a pest, to a locus of a pest, or to a plant susceptible to attack by a pest a combination of components A and B in a synergistically effective amount. In such applications mixtures of A and B will normally be applied in an insecticidally, acaricidally, nematicidally or molluscicidally effective amount. In application components A and B may be applied simultaneously or separately.

The active ingredient combinations are effective against harmful microorganisms, such as microorganisms, that cause phytopathogenic diseases, in particular against phytopathogenic fungi and bacteria. The active ingredient combinations are effective especially against phytopathogenic fungi belonging to the following classes: Ascomycetes (e.g. Venturia, Podosphaera, Erysiphe, Monilinia, Mycosphaerella, Uncinula); Basidiomycetes (e.g. the genus Hemileia, Rhizoctonia, Phakopsora, Puccinia, Ustilago, Tilletia); Fungi imperfecti (also known as Deuteromycetes; e.g. Botrytis, Helminthosporium, Rhynchosporium, Fusarium, Septoria, Cercospora, Alternaria, Pyricularia and Pseudocercosporella); Oomycetes (e.g. Phytophthora, Peronospora, Pseudoperonospora, Albugo, Bremia, Pythium, Pseudosclerospora, Plasmopara).

The mixtures of the present invention can be used to control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as animal pests. The animal pests which may be controlled by the use of the invention compounds include those animal pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies). The mixtures of the invention are particularly effective against insects, acarines and/or nematodes.

According to the invention “useful plants” typically comprise the following species of plants: grape vines; cereals, such as wheat, barley, rye or oats; beet, such as sugar beet or fodder beet; fruits, such as pomes, stone fruits or soft fruits, for example apples, pears, plums, peaches, almonds, cherries, strawberries, raspberries or blackberries; leguminous plants, such as beans, lentils, peas or soybeans; oil plants, such as rape, mustard, poppy, olives, sunflowers, coconut, castor oil plants, cocoa beans or groundnuts; cucumber plants, such as marrows, cucumbers or melons; fibre plants, such as cotton, flax, hemp or jute; citrus fruit, such as oranges, lemons, grapefruit or mandarins; vegetables, such as spinach, lettuce, asparagus, cabbages, carrots, onions, tomatoes, potatoes, cucurbits or paprika; lauraceae, such as avocados, cinnamon or camphor; maize; tobacco; nuts; coffee; sugar cane; tea; vines; hops; durian; bananas; natural rubber plants; turf or ornamentals, such as flowers, shrubs, broad-leaved trees or evergreens, for example conifers. This list does not represent any limitation.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). An example for a truncated toxin is a truncated CryIA(b), which is expressed in the Bt11 maize from Syngenta Seed SAS, as described below. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810)

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a CryIIIB(b1) toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

The term “useful plants” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Useful plants include soybean, corn, sugarcane, alfalfa, brassicas, oilseed rape (e.g. canola), potatoes (including sweet potatoes), cotton, rice, coffee, citrus, almonds, fruiting vegetables (e.g. tomatoes, pepper, chili, eggplant, cucumber, squash etc.), tea, bulb vegetables (e.g. onion, leek etc.), grapes, pome fruit (e.g. apples, pears etc.), and stone fruit (e.g. pears, plums etc.). Useful plants of elevated interest in connection with present invention are cereals; soybean; rice; oil seed rape; pome fruits; stone fruits; peanuts; coffee; tea; strawberries; turf; vines and vegetables, such as tomatoes, potatoes, cucurbits and lettuce.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of a plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds. Fungicides that are of particular interest for treating seeds include Fludioxonil, Thiabendazole, Sedaxane, Mefenoxam and Metalaxyl. Accordingly, in one embodiment component B is selected from Fludioxonil, Thiabendazole, Sedaxane, Mefenoxam and Metalaxyl.

A further aspect of the instant invention is a method of protecting natural substances of plant and/or animal origin, which have been taken from the natural life cycle, and/or their processed forms against attack of fungi and/or animal pests, which comprises applying to said natural substances of plant and/or animal origin or their processed forms a combination of components A and B in a synergistically effective amount.

According to the instant invention, the term “natural substances of plant origin, which have been taken from the natural life cycle” denotes plants or parts thereof which have been harvested from the natural life cycle and which are in the freshly harvested form. Examples of such natural substances of plant origin are stalks, leafs, tubers, seeds, fruits or grains. According to the instant invention, the term “processed form of a natural substance of plant origin” is understood to denote a form of a natural substance of plant origin that is the result of a modification process. Such modification processes can be used to transform the natural substance of plant origin in a more storable form of such a substance (a storage good). Examples of such modification processes are pre-drying, moistening, crushing, comminuting, grounding, compressing or roasting. Also falling under the definition of a processed form of a natural substance of plant origin is timber, whether in the form of crude timber, such as construction timber, electricity pylons and barriers, or in the form of finished articles, such as furniture or objects made from wood.

According to the instant invention, the term “natural substances of animal origin, which have been taken from the natural life cycle and/or their processed forms” is understood to denote material of animal origin such as skin, hides, leather, furs, hairs and the like.

The combinations according the present invention can prevent disadvantageous effects such as decay, discoloration or mould.

A preferred embodiment is a method of protecting natural substances of plant origin, which have been taken from the natural life cycle, and/or their processed forms against attack of fungi and/or animal pests, which comprises applying to said natural substances of plant and/or animal origin or their processed forms a combination of components A and B in a synergistically effective amount.

A further preferred embodiment is a method of protecting fruits, preferably pomes, stone fruits, soft fruits and citrus fruits, which have been taken from the natural life cycle, and/or their processed forms, which comprises applying to said fruits and/or their processed forms a combination of components A and B in a synergistically effective amount.

The combinations of the present invention may also be used in the field of protecting industrial material against attack of fungi. According to the instant invention, the term “industrial material” denotes non-living materials which have been prepared for use in industry. For example, industrial materials which are intended to be protected against attack of fungi can be glues, sizes, paper, board, textiles, carpets, leather, wood, constructions, paints, plastic articles, cooling lubricants, aquaeous hydraulic fluids and other materials which can be infested with, or decomposed by, microorganisms. Cooling and heating systems, ventilation and air conditioning systems and parts of production plants, for example cooling-water circuits, which may be impaired by multiplication of microorganisms may also be mentioned from amongst the materials to be protected. The combinations according the present invention can prevent disadvantageous effects such as decay, discoloration or mold.

The combinations of the present invention may also be used in the field of protecting technical material against attack of fungi. According to the instant invention, the term “technical material” includes paper; carpets; constructions; cooling and heating systems; ventilation and air conditioning systems and the like. The combinations according the present invention can prevent disadvantageous effects such as decay, discoloration or mold.

The combinations according to the present invention are particularly effective against powdery mildews; rusts; leafspot species; early blights and molds; especially against Septoria, Puccinia, Erysiphe, Pyrenophora and Tapesia in cereals; Phakopsora in soybeans; Hemileia in coffee; Phragmidium in roses; Alternaria in potatoes, tomatoes and cucurbits; Sclerotinia in turf, vegetables, sunflower and oil seed rape; black rot, red fire, powdery mildew, grey mold and dead arm disease in vine; Botrytis cinerea in fruits; Monilinia spp. in fruits and Penicillium spp. in fruits.

The combinations according to the present invention are furthermore particularly effective against seedborne and soilborne diseases, such as Alternaria spp., Ascochyta spp., Botrytis cinerea, Cercospora spp., Claviceps purpurea, Cochliobolus sativus, Colletotrichum spp., Epicoccum spp., Fusarium graminearum, Fusarium moniliforme, Fusarium oxysporum, Fusarium proliferatum, Fusarium solani, Fusarium subglutinans, Gaumannomyces graminis, Helminthosporium spp., Microdochium nivale, Phoma spp., Pyrenophora graminea, Pyricularia oryzae, Rhizoctonia solani, Rhizoctonia cerealis, Sclerotinia spp., Septoria spp., Sphacelotheca reilliana, Tilletia spp., Typhula incarnata, Urocystis occulta, Ustilago spp. or Verticillium spp.; in particular against pathogens of cereals, such as wheat, barley, rye or oats; maize; rice; cotton; soybean; turf; sugarbeet; oil seed rape; potatoes; pulse crops, such as peas, lentils or chickpea; and sunflower. The combinations according to the present invention are furthermore particularly effective against post harvest diseasese such as Botrytis cinerea, Colletotrichum musae, Curvularia lunata, Fusarium semitecum, Geotrichum candidum, Monilinia fructicola, Monilinia fructigena, Monilinia laxa, Mucor piriformis, Penicilium italicum, Penicilium solitum, Penicillium digitatum or Penicillium expansum in particular against pathogens of fruits, such as pomefruits, for example apples and pears, stone fruits, for example peaches and plums, citrus, melons, papaya, kiwi, mango, berries, for example strawberries, avocados, pomegranates and bananas, and nuts.

The combinations according to the invention are particularly useful for controlling the following plant diseases:

Alternaria species in fruit and vegetables,
Ascochyta species in pulse crops,
Botrytis cinerea in strawberries, tomatoes, sunflower, pulse crops, vegetables and grapes,
Cercospora arachidicola in peanuts,
Cochliobolus sativus in cereals,
Colletotrichum species in pulse crops,
Erysiphe species in cereals,
Erysiphe cichoracearum and Sphaerotheca fuliginea in cucurbits,
Fusarium species in cereals and maize,
Gäumannomyces graminis in cereals and lawns,
Helminthosporium species in maize, rice and potatoes,
Hemileia vastatrix on coffee,
Microdochium species in wheat and rye,
Phakopsora species in soybean,
Puccinia species in cereals, broadleaf crops and perrenial plants,
Pseudocercosporella species in cereals,
Phragmidium mucronatum in roses,
Podosphaera species in fruits,
Pyrenophora species in barley,
Pyricularia oryzae in rice,
Ramularia collo-cygni in barley,
Rhizoctonia species in cotton, soybean, cereals, maize, potatoes, rice and lawns,
Rhynchosporium secalis in barley and rye,
Sclerotinia species in lawns, lettuce, vegetables and oil seed rape,
Septoria species in cereals, soybean and vegetables,
Sphacelotheca reilliana in maize,
Tilletia species in cereals,
Uncinula necator, Guignardia bidwellii and Phomopsis viticola in vines,
Urocystis occulta in rye,
Ustilago species in cereals and maize,
Venturia species in fruits,
Monilinia species on fruits,
Penicillium species on citrus and apples.

The combinations according to the present invention are furthermore particularly effective against the following animal pests: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus lotus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulfureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans (vinegar eelworm), Trichostrongylus spp. (gastro intestinal nematodes) and Deroceras reticulatum (slug).

The amount of a combination of the invention to be applied, will depend on various factors, such as the compounds employed; the subject of the treatment, such as, for example plants, soil or seeds; the type of treatment, such as, for example spraying, dusting or seed dressing; the purpose of the treatment, such as, for example prophylactic or therapeutic; the type of fungi and/or animal pest to be controlled or the application time.

The mixtures comprising a compound of formula I, e.g. those selected from table A, and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Table A and the active ingredients as described above is not essential for working the present invention.

Synergistic activity is present when the fungicidal and/or animal pesticidal activity of the composition of A+B is greater than the sum of the fungicidal and/or pesticidal activities of A and B.

The method of the invention comprises applying to the useful plants, the locus thereof or propagation material thereof in admixture or separately, a synergistically effective aggregate amount of a component A and a component B.

Some of said combinations according to the invention have a systemic action and can be used as foliar, soil and seed treatment pesticides.

With the combinations according to the invention it is possible to inhibit or destroy the phytopathogenic microorganisms and/or animal pests which occur in plants or in parts of plants (fruit, blossoms, leaves, stems, tubers, roots) in different useful plants, while at the same time the parts of plants which grow later are also protected from attack by phytopathogenic microorganisms and/or animal pests.

The combinations of the present invention are of particular interest for controlling a large number of fungi and/or animal pests in various useful plants or their seeds, especially in field crops such as potatoes, tobacco and sugarbeets, and wheat, rye, barley, oats, rice, maize, lawns, cotton, soybeans, oil seed rape, pulse crops, sunflower, coffee, sugarcane, fruit and ornamentals in horticulture and viticulture, in vegetables such as cucumbers, beans and cucurbits.

The combinations according to the invention are applied by treating the fungi and/or animal pests, the useful plants, the locus thereof, the propagation material thereof, the natural substances of plant and/or animal origin, which have been taken from the natural life cycle, and/or their processed forms, or the industrial materials threatened by fungus and/or animal pests, attack with a combination of components A and B in a synergistically effective amount.

The combinations according to the invention may be applied before or after infection or contamination of the useful plants, the propagation material thereof, the natural substances of plant and/or animal origin, which have been taken from the natural life cycle, and/or their processed forms, or the industrial materials by the fungi and/or animal pests.

When applied to the useful plants the compound of formula I is applied at a rate of 1 to 500 g a.i./ha in association with 1 to 5000 g a.i./ha, particularly 1 to 2000 g a.i./ha, of a compound of component B, depending on the class of chemical employed as component B.

Generally for plant propagation material, such as seed treatment, application rates can vary from 0.001 to 10 g/kg of seeds of active ingredients for compounds of formula I. When the combinations of the present invention are used for treating seed, rates of 0.001 to 5 g of a compound of formula I per kg of seed, preferably from 0.01 to 1 g per kg of seed, and 0.001 to 50 g of a compound of component B, per kg of seed, preferably from 0.01 to 10 g per kg of seed, are generally sufficient.

The weight ratio of A to B may generally be between 1000:1 and 1:1000. In other embodiments that weight ratio of A to B may be between 500:1 to 1:500, for example between 100:1 to 1:100, for example between 1:50 to 50:1, for example 1:20 to 20:1.

The invention also provides pesticidal mixtures comprising a combination of components A and B as mentioned above in a synergistically effective amount, together with an agriculturally acceptable carrier, and optionally a surfactant.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules. A typical a tank-mix formulation for seed treatment application comprises 0.25 to 80%, especially 1 to 75%, of the desired ingredients, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mix formulation. A typical pre-mix formulation for seed treatment application comprises 0.5 to 99.9%, especially 1 to 95%, of the desired ingredients, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula I together with a compound of component B, and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

A synergistic effect exists whenever the action of an active ingredient combination is greater than the sum of the actions of the individual components.

The action to be expected E for a given active ingredient combination obeys the so-called COLBY formula and can be calculated as follows (COLBY, S.R. “Calculating synergistic and antagonistic responses of herbicide combination”. Weeds, Vol. 15, pages 20-22; 1967):

ppm=milligrams of active ingredient (=a.i.) per liter of spray mixture
X=% action by active ingredient A) using p ppm of active ingredient
Y=% action by active ingredient B) using q ppm of active ingredient.

According to COLBY, the expected (additive) action of active ingredients A)+B) using p+q ppm of active ingredient is

$$E = X + Y - \frac{X \cdot Y}{100}$$

If the action actually observed (O) is greater than the expected action (E), then the action of the combination is super-additive, i.e. there is a synergistic effect. In mathematical terms the synergism factor SF corresponds to O/E. In the agricultural practice an SF of ≧1.2 indicates significant improvement over the purely complementary addition of activities (expected activity), while an SF of ≦0.9 in the practical application routine signals a loss of activity compared to the expected activity.

Tables 1 to 198 show mixtures and compositions of the present invention demonstrating control on a wide range of fungi. In some cases the presence of the compound of formula I notably increases the fungicidal action of the fungicide. Given that the compounds of formula I does not usually have any fungicidal action of their own, this is a surprising effect. Noteworthy are mixtures comprising A1 and Azoxystrobin, Isopyrazam, Chlorothalonil, Cyproconazole, Difenoconaozle, Mandipropamid, Mefenoxam, a compound of formula III or a compound of formula IV; mixtures comprising A5 and Azoxystrobin, Isopyrazam, Chlorothalonil, Cyproconazole, Difenoconaozle, Mandipropamid, Mefenoxam, a compound of formula III or a compound of formula IV; mixtures comprising A6 and Azoxystrobin, Isopyrazam, Chlorothalonil, Cyproconazole, Difenoconaozle, Mandipropamid, Mefenoxam, a compound of formula III or a compound of formula IV; mixtures comprising A6 and A7 and Azoxystrobin, Isopyrazam, Chlorothalonil, Cyproconazole, Difenoconaozle, Mandipropamid, Mefenoxam, a compound of formula III or a compound of formula IV, mixtures comprising A8 and Azoxystrobin, Isopyrazam, Chlorothalonil, Cyproconazole, Difenoconaozle, Mandipropamid, Mefenoxam, a compound of formula III or a compound of formula IV.

Pythium ultimum (Damping Off):

Mycelial fragments of the fungus, prepared from a fresh liquid culture, were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 48 hours. Application rates are shown in the Tables.

TABLE 1

| | | AVERAGE | | | |
|---|---|---|---|---|---|
| PPM Al | | DEAD IN % | | | |
| | Azoxy- | | Azoxy- | EXPECTED | ACTUAL |
| A1 | strobin | A1 | strobin | CONTROL | CONTROL |
| 20 | 0.5 | 0 | 20 | 20 | 20 |
| 20 | 1 | 0 | 20 | 20 | 20 |
| 10 | 0.5 | 0 | 20 | 20 | 20 |
| 5 | 0.25 | 0 | 0 | 0 | 20* |
| 20 | 2 | 0 | 50 | 50 | 50 |
| 10 | 1 | 0 | 20 | 20 | 20 |
| 5 | 0.5 | 0 | 20 | 20 | 20 |
| 10 | 2 | 0 | 50 | 50 | 50 |
| 5 | 1 | 0 | 20 | 20 | 20 |
| 2.5 | 0.5 | 0 | 20 | 20 | 20 |
| 1.25 | 0.25 | 0 | 0 | 0 | 20* |
| 5 | 2 | 0 | 50 | 50 | 50 |
| 2.5 | 1 | 0 | 20 | 20 | 20 |
| 1.25 | 0.5 | 0 | 20 | 20 | 20 |
| 0.625 | 0.25 | 0 | 0 | 0 | 20* |
| 0.3125 | 0.125 | 0 | 0 | 0 | 20* |

TABLE 2

| | | AVERAGE | | | |
|---|---|---|---|---|---|
| PPM Al | | DEAD IN % | | | |
| | Chloro- | | Chloro- | EXPECTED | ACTUAL |
| A1 | thalonil | A1 | thalonil | CONTROL | CONTROL |
| 20 | 2 | 0 | 20 | 20 | 0 |
| 10 | 2 | 0 | 20 | 20 | 0 |
| 5 | 2 | 0 | 20 | 20 | 0 |

TABLE 3

| | | | | EX- | |
|---|---|---|---|---|---|
| | | | | PECT- | ACTU- |
| | | AVERAGE | | ED | AL |
| PPM Al | | DEAD IN % | | CON- | CON- |
| A1 | Mefenoxam | A1 | Mefenoxam | TROL | TROL |
| 20 | 0.5 | 0 | 90 | 90 | 100* |
| 10 | 0.25 | 0 | 70 | 70 | 70 |
| 5 | 0.125 | 0 | 50 | 50 | 70* |
| 2.5 | 0.0625 | 0 | 20 | 20 | 20 |
| 1.25 | 0.03125 | 0 | 20 | 20 | 20 |
| 0.625 | 0.015625 | 0 | 20 | 20 | 20 |
| 10 | 0.5 | 0 | 90 | 90 | 90 |
| 5 | 0.25 | 0 | 70 | 70 | 90* |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 0.3125 | 0.015625 | 0 | 20 | 20 | 20 |
| 5 | 0.5 | 0 | 90 | 90 | 90 |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 0.15625 | 0.015625 | 0 | 20 | 20 | 20 |
| 2.5 | 0.5 | 0 | 90 | 90 | 90 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 0.078125 | 0.015625 | 0 | 20 | 20 | 0 |
| 1.25 | 0.5 | 0 | 90 | 90 | 90 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 20 | 20 | 20 |

TABLE 4

| | | AVERAGE | | | ACTUAL |
|---|---|---|---|---|---|
| PPM AI | | DEAD IN % | | EXPECTED | CON- |
| A5 | Azoxystrobin | A5 | Azoxystrobin | CONTROL | TROL |
| 20 | 0.5 | 0 | 50 | 50 | 50 |
| 10 | 0.25 | 0 | 20 | 20 | 20 |
| 20 | 1 | 0 | 70 | 70 | 70 |
| 10 | 0.5 | 0 | 50 | 50 | 20 |
| 5 | 0.25 | 0 | 20 | 20 | 0 |
| 10 | 1 | 0 | 70 | 70 | 70 |
| 5 | 0.5 | 0 | 50 | 50 | 20 |
| 2.5 | 0.25 | 0 | 20 | 20 | 0 |
| 5 | 1 | 0 | 70 | 70 | 90* |
| 2.5 | 0.5 | 0 | 50 | 50 | 50 |
| 1.25 | 0.25 | 0 | 20 | 20 | 20 |
| 5 | 2 | 0 | 100 | 100 | 100 |
| 2.5 | 1 | 0 | 70 | 70 | 70 |
| 1.25 | 0.5 | 0 | 50 | 50 | 50 |
| 0.625 | 0.25 | 0 | 20 | 20 | 20 |

TABLE 5

| | | AVERAGE | | | |
|---|---|---|---|---|---|
| PPM AI | | DEAD IN % | | EXPECTED | ACTUAL |
| A5 | Chlorothalonil | A5 | Chlorothalonil | CONTROL | CONTROL |
| 20 | 2 | 0 | 50 | 50 | 50 |
| 10 | 2 | 0 | 50 | 50 | 50 |
| 5 | 1 | 0 | 0 | 0 | 20* |
| 5 | 2 | 0 | 50 | 50 | 70* |

TABLE 6

| | | AVERAGE | | | ACTUAL |
|---|---|---|---|---|---|
| PPM AI | | DEAD IN % | | EXPECTED | CON- |
| A5 | Mefenoxam | A5 | Mefenoxam | CONTROL | TROL |
| 10 | 0.25 | 0 | 90 | 90 | 90 |
| 5 | 0.125 | 0 | 70 | 70 | 70 |
| 2.5 | 0.0625 | 0 | 50 | 50 | 50 |
| 1.25 | 0.03125 | 0 | 50 | 50 | 50 |
| 0.625 | 0.015625 | 0 | 20 | 20 | 20 |
| 5 | 0.25 | 0 | 90 | 90 | 90 |
| 2.5 | 0.125 | 0 | 70 | 70 | 70 |
| 1.25 | 0.0625 | 0 | 50 | 50 | 50 |
| 0.625 | 0.03125 | 0 | 50 | 50 | 50 |
| 0.3125 | 0.015625 | 0 | 20 | 20 | 20 |
| 2.5 | 0.25 | 0 | 90 | 90 | 90 |
| 1.25 | 0.125 | 0 | 70 | 70 | 70 |
| 0.625 | 0.0625 | 0 | 50 | 50 | 50 |
| 0.3125 | 0.03125 | 0 | 50 | 50 | 50 |
| 0.15625 | 0.015625 | 0 | 20 | 20 | 20 |
| 1.25 | 0.25 | 0 | 90 | 90 | 90 |
| 0.625 | 0.125 | 0 | 70 | 70 | 70 |
| 0.3125 | 0.0625 | 0 | 50 | 50 | 50 |
| 0.15625 | 0.03125 | 0 | 50 | 50 | 20 |
| 0.078125 | 0.015625 | 0 | 20 | 20 | 20 |
| 0.625 | 0.25 | 0 | 90 | 90 | 90 |
| 0.3125 | 0.125 | 0 | 70 | 70 | 70 |
| 0.15625 | 0.0625 | 0 | 50 | 50 | 50 |
| 0.078125 | 0.03125 | 0 | 50 | 50 | 20 |
| 0.0390625 | 0.015625 | 0 | 20 | 20 | 20 |

TABLE 7

| | | AVERAGE | | | |
|---|---|---|---|---|---|
| PPM AI | | DEAD IN % | | EXPECTED | ACTUAL |
| A6 | Azoxystrobin | A6 | Azoxystrobin | CONTROL | CONTROL |
| 20 | 0.5 | 0 | 20 | 20 | 0 |
| 10 | 0.25 | 0 | 20 | 20 | 0 |
| 20 | 1 | 0 | 20 | 20 | 20 |
| 10 | 0.5 | 0 | 20 | 20 | 0 |
| 5 | 0.25 | 0 | 20 | 20 | 0 |
| 20 | 2 | 0 | 20 | 20 | 20 |
| 10 | 1 | 0 | 20 | 20 | 20 |
| 5 | 0.5 | 0 | 20 | 20 | 0 |
| 2.5 | 0.25 | 0 | 20 | 20 | 0 |
| 10 | 2 | 0 | 20 | 20 | 20 |
| 5 | 1 | 0 | 20 | 20 | 20 |
| 2.5 | 0.5 | 0 | 20 | 20 | 0 |
| 1.25 | 0.25 | 0 | 20 | 20 | 0 |

TABLE 8

| | | AVERAGE | | | |
|---|---|---|---|---|---|
| PPM AI | | DEAD IN % | | EXPECTED | ACTUAL |
| A6 | Chlorothalonil | A6 | Chlorothalonil | CONTROL | CONTROL |
| 20 | 2 | 0 | 50 | 50 | 90* |
| 10 | 1 | 0 | 0 | 0 | 50* |
| 10 | 2 | 0 | 50 | 50 | 50 |
| 5 | 1 | 0 | 0 | 0 | 50* |
| 5 | 2 | 0 | 50 | 50 | 70* |
| 2.5 | 1 | 0 | 0 | 0 | 70* |

TABLE 9

| | | AVERAGE | | | |
|---|---|---|---|---|---|
| PPM AI | | DEAD IN % | | EXPECTED | ACTUAL |
| A6 | Mefenoxam | A6 | Mefenoxam | CONTROL | CONTROL |
| 10 | 0.25 | 0 | 70 | 70 | 70 |
| 5 | 0.125 | 0 | 70 | 70 | 70 |
| 2.5 | 0.0625 | 0 | 50 | 50 | 50 |
| 10 | 0.5 | 0 | 100 | 100 | 90 |
| 5 | 0.25 | 0 | 70 | 70 | 90* |
| 2.5 | 0.125 | 0 | 70 | 70 | 70 |
| 1.25 | 0.0625 | 0 | 50 | 50 | 50 |
| 0.625 | 0.03125 | 0 | 20 | 20 | 20 |
| 5 | 0.5 | 0 | 100 | 100 | 90 |
| 2.5 | 0.25 | 0 | 70 | 70 | 90* |
| 1.25 | 0.125 | 0 | 70 | 70 | 70 |
| 0.625 | 0.0625 | 0 | 50 | 50 | 50 |
| 0.3125 | 0.03125 | 0 | 20 | 20 | 0 |
| 2.5 | 0.5 | 0 | 100 | 100 | 90 |
| 1.25 | 0.25 | 0 | 70 | 70 | 70 |
| 0.625 | 0.125 | 0 | 70 | 70 | 50 |
| 0.3125 | 0.0625 | 0 | 50 | 50 | 50 |
| 0.15625 | 0.03125 | 0 | 20 | 20 | 20 |
| 0.625 | 0.25 | 0 | 70 | 70 | 90* |
| 0.3125 | 0.125 | 0 | 70 | 70 | 70 |
| 0.15625 | 0.0625 | 0 | 50 | 50 | 50 |
| 0.078125 | 0.03125 | 0 | 20 | 20 | 20 |

TABLE 10

| | | AVERAGE | | | |
|---|---|---|---|---|---|
| PPM AI | | DEAD IN % | | EXPECTED | ACTUAL |
| A8 | Azoxystrobin | A8 | Azoxystrobin | CONTROL | CONTROL |
| 20 | 0.5 | 0 | 20 | 20 | 20 |
| 20 | 1 | 0 | 20 | 20 | 20 |
| 10 | 0.5 | 0 | 20 | 20 | 20 |
| 20 | 2 | 0 | 20 | 20 | 20 |
| 10 | 1 | 0 | 20 | 20 | 20 |
| 5 | 0.5 | 0 | 20 | 20 | 0 |
| 10 | 2 | 0 | 20 | 20 | 20 |
| 5 | 1 | 0 | 20 | 20 | 20 |
| 2.5 | 0.5 | 0 | 20 | 20 | 0 |
| 5 | 2 | 0 | 20 | 20 | 20 |
| 2.5 | 1 | 0 | 20 | 20 | 20 |
| 1.25 | 0.5 | 0 | 20 | 20 | 0 |

TABLE 11

| | | AVERAGE | | | |
|---|---|---|---|---|---|
| PPM AI | | DEAD IN % | | EXPECTED | ACTUAL |
| A8 | Chlorothalonil | A8 | Chlorothalonil | CONTROL | CONTROL |
| 20 | 1 | 0 | 0 | 0 | 90* |
| 5 | 0.25 | 0 | 0 | 0 | ne |
| 20 | 2 | 0 | 70 | 70 | 70 |
| 10 | 2 | 0 | 70 | 70 | 50 |
| 5 | 1 | 0 | 0 | 0 | 20* |
| 2.5 | 0.5 | 0 | 0 | 0 | 20* |
| 5 | 2 | 0 | 70 | 70 | 50 |
| 2.5 | 1 | 0 | 0 | 0 | 20* |

TABLE 12

| | | AVERAGE | | | |
|---|---|---|---|---|---|
| PPM AI | | DEAD IN % | | EXPECTED | ACTUAL |
| A8 | Mefenoxam | A8 | Mefenoxam | CONTROL | CONTROL |
| 20 | 0.5 | 0 | 90 | 90 | 90 |
| 10 | 0.25 | 0 | 70 | 70 | 70 |
| 5 | 0.125 | 0 | 70 | 70 | 50 |
| 2.5 | 0.0625 | 0 | 50 | 50 | 20 |
| 1.25 | 0.03125 | 0 | 20 | 20 | 20 |
| 10 | 0.5 | 0 | 90 | 90 | 100* |
| 5 | 0.25 | 0 | 70 | 70 | 90* |
| 2.5 | 0.125 | 0 | 70 | 70 | 50 |
| 1.25 | 0.0625 | 0 | 50 | 50 | 50 |
| 0.625 | 0.03125 | 0 | 20 | 20 | 20 |
| 0.3125 | 0.015625 | 0 | 0 | 0 | 20* |
| 5 | 0.5 | 0 | 90 | 90 | 90 |
| 2.5 | 0.25 | 0 | 70 | 70 | 70 |
| 1.25 | 0.125 | 0 | 70 | 70 | 50 |
| 0.625 | 0.0625 | 0 | 50 | 50 | 50 |
| 0.3125 | 0.03125 | 0 | 20 | 20 | 20 |
| 2.5 | 0.5 | 0 | 90 | 90 | 90 |
| 1.25 | 0.25 | 0 | 70 | 70 | 90* |
| 0.625 | 0.125 | 0 | 70 | 70 | 70 |
| 0.3125 | 0.0625 | 0 | 50 | 50 | 50 |
| 0.15625 | 0.03125 | 0 | 20 | 20 | 20 |
| 5 | 2 | 0 | 100 | 100 | 100 |
| 2.5 | 1 | 0 | 100 | 100 | 100 |
| 1.25 | 0.5 | 0 | 90 | 90 | 90 |
| 0.625 | 0.25 | 0 | 70 | 70 | 70 |
| 0.3125 | 0.125 | 0 | 70 | 70 | 50 |
| 0.15625 | 0.0625 | 0 | 50 | 50 | 50 |
| 0.078125 | 0.03125 | 0 | 20 | 20 | 20 |

Rhizoctonia solani (Foot Rot, Damping-Off):

Mycelial fragments of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 48 hours. Application rates are shown in the Tables.

TABLE 13

| | | AVERAGE | | | ACTUAL |
|---|---|---|---|---|---|
| PPM AI | | DEAD IN % | | EXPECTED | CON- |
| A1 | Azoxystrobin | A1 | Azoxystrobin | CONTROL | TROL |
| 20 | 0.5 | 0 | 20 | 20 | 0 |
| 10 | 0.25 | 0 | 20 | 20 | 0 |
| 5 | 0.125 | 0 | 20 | 20 | 0 |
| 2.5 | 0.0625 | 0 | 20 | 20 | 0 |
| 20 | 1 | 0 | 20 | 20 | 20 |
| 10 | 0.5 | 0 | 20 | 20 | 20 |
| 5 | 0.25 | 0 | 20 | 20 | 20 |
| 2.5 | 0.125 | 0 | 20 | 20 | 20 |
| 1.25 | 0.0625 | 0 | 20 | 20 | 0 |
| 20 | 2 | 0 | 20 | 20 | 20 |
| 10 | 1 | 0 | 20 | 20 | 20 |
| 5 | 0.5 | 0 | 20 | 20 | 20 |
| 2.5 | 0.25 | 0 | 20 | 20 | 20 |
| 1.25 | 0.125 | 0 | 20 | 20 | 20 |
| 0.625 | 0.0625 | 0 | 20 | 20 | 20 |
| 10 | 2 | 0 | 20 | 20 | 20 |
| 5 | 1 | 0 | 20 | 20 | 20 |
| 2.5 | 0.5 | 0 | 20 | 20 | 20 |
| 1.25 | 0.25 | 0 | 20 | 20 | 20 |
| 0.625 | 0.125 | 0 | 20 | 20 | 20 |
| 0.3125 | 0.0625 | 0 | 20 | 20 | 0 |
| 5 | 2 | 0 | 20 | 20 | 20 |
| 2.5 | 1 | 0 | 20 | 20 | 20 |
| 1.25 | 0.5 | 0 | 20 | 20 | 20 |
| 0.625 | 0.25 | 0 | 20 | 20 | 20 |
| 0.3125 | 0.125 | 0 | 20 | 20 | 20 |

TABLE 14

| | | AVERAGE | | | ACTUAL |
|---|---|---|---|---|---|
| PPM AI | | DEAD IN % | | EXPECTED | CON- |
| A1 | Isopyrazam | A1 | Isopyrazam | CONTROL | TROL |
| 10 | 0.25 | 0 | 90 | 90 | 100* |
| 5 | 0.125 | 0 | 70 | 70 | 90* |
| 2.5 | 0.0625 | 0 | 50 | 50 | 50 |
| 5 | 0.25 | 0 | 90 | 90 | 100* |
| 2.5 | 0.125 | 0 | 70 | 70 | 90* |
| 1.25 | 0.0625 | 0 | 50 | 50 | 50 |
| 2.5 | 0.25 | 0 | 90 | 90 | 90 |
| 1.25 | 0.125 | 0 | 70 | 70 | 90* |
| 0.625 | 0.0625 | 0 | 50 | 50 | 70* |
| 0.3125 | 0.03125 | 0 | 0 | 0 | 20* |
| 0.15625 | 0.015625 | 0 | 0 | 0 | 20* |
| 1.25 | 0.25 | 0 | 90 | 90 | 100* |
| 0.625 | 0.125 | 0 | 70 | 70 | 90* |
| 0.3125 | 0.0625 | 0 | 50 | 50 | 90* |
| 0.15625 | 0.03125 | 0 | 0 | 0 | 90* |
| 0.078125 | 0.015625 | 0 | 0 | 0 | 70* |
| 0.0390625 | 0.0078125 | 0 | 0 | 0 | 50* |
| 0.625 | 0.25 | 0 | 90 | 90 | 100* |
| 0.3125 | 0.125 | 0 | 70 | 70 | 100* |
| 0.15625 | 0.0625 | 0 | 50 | 50 | 90* |
| 0.078125 | 0.03125 | 0 | 0 | 0 | 90* |
| 0.0390625 | 0.015625 | 0 | 0 | 0 | 70* |

TABLE 15

| | | AVERAGE | | | |
|---|---|---|---|---|---|
| PPM AI | | DEAD IN % | | EXPECTED | ACTUAL |
| A1 | Chlorothalonil | A1 | Chlorothalonil | CONTROL | CONTROL |
| 10 | 2 | 0 | 0 | 0 | 90* |

TABLE 16

| | | AVERAGE | | | |
|---|---|---|---|---|---|
| PPM AI | | DEAD IN % | | EXPECTED | ACTUAL |
| A1 | Cyproconazole | A1 | Cyproconazole | CONTROL | CONTROL |
| 20 | 0.5 | 0 | 0 | 0 | 20* |
| 20 | 1 | 0 | 20 | 20 | 50* |
| 20 | 2 | 0 | 70 | 70 | 70 |
| 10 | 1 | 0 | 20 | 20 | 20 |
| 10 | 2 | 0 | 70 | 70 | 70 |
| 5 | 1 | 0 | 20 | 20 | 20 |
| 5 | 2 | 0 | 70 | 70 | 70 |
| 2.5 | 1 | 0 | 20 | 20 | 20 |
| 1.25 | 0.5 | 0 | 0 | 0 | 20* |

TABLE 17

| | | AVERAGE | | | |
|---|---|---|---|---|---|
| | | DEAD IN % | | | |
| PPM AI | | | COM- | | |
| | COMPOUND | | POUND | EXPECTED | ACTUAL |
| A1 | III | A1 | III | CONTROL | CONTROL |
| 1.25 | 0.03125 | 0 | 90 | 90 | 90 |
| 0.625 | 0.015625 | 0 | 20 | 20 | 50* |
| 0.625 | 0.03125 | 0 | 90 | 90 | 90 |
| 0.3125 | 0.015625 | 0 | 20 | 20 | 20 |
| 0.3125 | 0.03125 | 0 | 90 | 90 | 100* |
| 0.15625 | 0.015625 | 0 | 20 | 20 | 90* |
| 0.078125 | 0.0078125 | 0 | 0 | 0 | 50* |
| 0.15625 | 0.03125 | 0 | 90 | 90 | 100* |
| 0.078125 | 0.015625 | 0 | 20 | 20 | 100* |
| 0.0390625 | 0.0078125 | 0 | 0 | 0 | 100* |
| 0.078125 | 0.03125 | 0 | 90 | 90 | 100* |
| 0.0390625 | 0.015625 | 0 | 20 | 20 | 100* |

TABLE 18

| | | AVERAGE | | | |
|---|---|---|---|---|---|
| PPM AI | | DEAD IN % | | EXPECTED | ACTUAL |
| A5 | Isopyrazam | A5 | Isopyrazam | CONTROL | CONTROL |
| 10 | 0.25 | 0 | 90 | 90 | 100* |
| 5 | 0.125 | 0 | 70 | 70 | 70 |
| 2.5 | 0.0625 | 0 | 20 | 20 | 20 |
| 5 | 0.25 | 0 | 90 | 90 | 100* |
| 2.5 | 0.125 | 0 | 70 | 70 | 70 |
| 1.25 | 0.0625 | 0 | 20 | 20 | 50* |
| 2.5 | 0.25 | 0 | 90 | 90 | 100* |
| 1.25 | 0.125 | 0 | 70 | 70 | 70 |
| 0.625 | 0.0625 | 0 | 20 | 20 | 20 |
| 1.25 | 0.25 | 0 | 90 | 90 | 100* |
| 0.625 | 0.125 | 0 | 70 | 70 | 90* |
| 0.3125 | 0.0625 | 0 | 20 | 20 | 50* |
| 0.625 | 0.25 | 0 | 90 | 90 | 100* |
| 0.3125 | 0.125 | 0 | 70 | 70 | 90* |
| 0.15625 | 0.0625 | 0 | 20 | 20 | 20 |

TABLE 19

| | | AVERAGE | | | |
|---|---|---|---|---|---|
| PPM AI | | DEAD IN % | | EXPECTED | ACTUAL |
| A5 | Chlorothalonil | A5 | Chlorothalonil | CONTROL | CONTROL |
| 2.5 | 1 | 0 | 0 | 0 | 20* |

TABLE 20

| | | AVERAGE | | | |
|---|---|---|---|---|---|
| PPM AI | | DEAD IN % | | EXPECTED | ACTUAL |
| A5 | Cyproconazole | A5 | Cyproconazole | CONTROL | CONTROL |
| 20 | 1 | 0 | 90 | 90 | 90 |
| 10 | 0.5 | 0 | 20 | 20 | 20 |
| 10 | 1 | 0 | 90 | 90 | 90 |
| 5 | 0.5 | 0 | 20 | 20 | 50* |
| 5 | 1 | 0 | 90 | 90 | 90 |
| 2.5 | 0.5 | 0 | 20 | 20 | 50* |
| 2.5 | 1 | 0 | 90 | 90 | 90 |
| 1.25 | 0.5 | 0 | 20 | 20 | 50* |

TABLE 21

| | | AVERAGE | | | |
|---|---|---|---|---|---|
| PPM AI | | DEAD IN % | | EXPECTED | ACTUAL |
| A5 | Difenoconazole | A5 | Difenoconazole | CONTROL | CONTROL |
| 20 | 1 | 0 | 50 | 50 | 90* |
| 10 | 1 | 0 | 50 | 50 | 90* |
| 5 | 1 | 0 | 50 | 50 | 90* |
| 2.5 | 1 | 0 | 50 | 50 | 90* |

TABLE 22

| PPM AI | | AVERAGE | | | |
|---|---|---|---|---|---|
| | COM- | DEAD IN % | | | |
| | POUND | | COMPOUND | EXPECTED | ACTUAL |
| A5 | III | A5 | III | CONTROL | CONTROL |
| 1.25 | 0.03125 | 0 | 90 | 90 | 90 |
| 0.625 | 0.015625 | 0 | 70 | 70 | 0 |
| 0.625 | 0.03125 | 0 | 90 | 90 | 90 |
| 0.3125 | 0.015625 | 0 | 70 | 70 | 70 |
| 0.3125 | 0.03125 | 0 | 90 | 90 | 100* |
| 0.15625 | 0.015625 | 0 | 70 | 70 | 50 |
| 0.15625 | 0.03125 | 0 | 90 | 90 | 90 |
| 0.078125 | 0.015625 | 0 | 70 | 70 | 70 |
| 0.078125 | 0.03125 | 0 | 90 | 90 | 100* |
| 0.0390625 | 0.015625 | 0 | 70 | 70 | 70 |

TABLE 23

| | | AVERAGE | | | ACTUAL |
|---|---|---|---|---|---|
| PPM AI | | DEAD IN % | | EXPECTED | CON- |
| A6 | Azoxystrobin | A6 | Azoxystrobin | CONTROL | TROL |
| 20 | 0.5 | 0 | 20 | 20 | 20 |
| 10 | 0.25 | 0 | 20 | 20 | 20 |
| 5 | 0.125 | 0 | 20 | 20 | 20 |
| 20 | 1 | 0 | 20 | 20 | 20 |
| 10 | 0.5 | 0 | 20 | 20 | 20 |
| 5 | 0.25 | 0 | 20 | 20 | 20 |
| 2.5 | 0.125 | 0 | 20 | 20 | 20 |
| 20 | 2 | 0 | 20 | 20 | 20 |
| 10 | 1 | 0 | 20 | 20 | 20 |
| 5 | 0.5 | 0 | 20 | 20 | 20 |
| 2.5 | 0.25 | 0 | 20 | 20 | 20 |
| 1.25 | 0.125 | 0 | 20 | 20 | 20 |
| 10 | 2 | 0 | 20 | 20 | 20 |
| 5 | 1 | 0 | 20 | 20 | 20 |
| 2.5 | 0.5 | 0 | 20 | 20 | 20 |
| 1.25 | 0.25 | 0 | 20 | 20 | 20 |
| 0.625 | 0.125 | 0 | 20 | 20 | 20 |
| 5 | 2 | 0 | 20 | 20 | 20 |
| 2.5 | 1 | 0 | 20 | 20 | 20 |
| 1.25 | 0.5 | 0 | 20 | 20 | 20 |
| 0.625 | 0.25 | 0 | 20 | 20 | 20 |
| 0.3125 | 0.125 | 0 | 20 | 20 | 20 |

TABLE 24

| | | AVERAGE | | | |
|---|---|---|---|---|---|
| PPM AI | | DEAD IN % | | EXPECTED | ACTUAL |
| A6 | Isopyrazam | A6 | Isopyrazam | CONTROL | CONTROL |
| 5 | 0.125 | 0 | 90 | 90 | 90 |
| 2.5 | 0.0625 | 0 | 70 | 70 | 70 |
| 1.25 | 0.03125 | 0 | 50 | 50 | 50 |
| 2.5 | 0.125 | 0 | 90 | 90 | 90 |
| 1.25 | 0.0625 | 0 | 70 | 70 | 70 |
| 0.625 | 0.03125 | 0 | 50 | 50 | 50 |
| 1.25 | 0.125 | 0 | 90 | 90 | 90 |
| 0.625 | 0.0625 | 0 | 70 | 70 | 70 |
| 0.3125 | 0.03125 | 0 | 50 | 50 | 50 |
| 0.625 | 0.125 | 0 | 90 | 90 | 90 |
| 0.3125 | 0.0625 | 0 | 70 | 70 | 70 |
| 0.15625 | 0.03125 | 0 | 50 | 50 | 50 |
| 0.3125 | 0.125 | 0 | 90 | 90 | 90 |
| 0.15625 | 0.0625 | 0 | 70 | 70 | 70 |
| 0.078125 | 0.03125 | 0 | 50 | 50 | 0 |

TABLE 25

| | | AVERAGE | | | |
|---|---|---|---|---|---|
| PPM AI | | DEAD IN % | | EXPECTED | ACTUAL |
| A6 | Chlorothalonil | A6 | Chlorothalonil | CONTROL | CONTROL |
| 20 | 0.5 | 0 | 70 | 70 | 100* |
| 20 | 1 | 0 | 90 | 90 | 100* |
| 10 | 0.5 | 0 | 70 | 70 | 100* |
| 10 | 1 | 0 | 90 | 90 | 100* |
| 5 | 0.5 | 0 | 70 | 70 | 100* |
| 5 | 1 | 0 | 90 | 90 | 100* |
| 2.5 | 0.5 | 0 | 70 | 70 | 90* |
| 2.5 | 1 | 0 | 90 | 90 | 100* |
| 1.25 | 0.5 | 0 | 70 | 70 | 90* |

TABLE 26

| | | AVERAGE | | | ACTUAL |
|---|---|---|---|---|---|
| PPM AI | | DEAD IN % | | EXPECTED | CON- |
| A6 | Cyproconazole | A6 | Cyproconazole | CONTROL | TROL |
| 20 | 0.5 | 0 | 50 | 50 | 50 |
| 10 | 0.25 | 0 | 20 | 20 | 20 |
| 20 | 1 | 0 | 90 | 90 | 100* |
| 10 | 0.5 | 0 | 50 | 50 | 50 |
| 5 | 0.25 | 0 | 20 | 20 | 0 |
| 10 | 1 | 0 | 90 | 90 | 100* |
| 5 | 0.5 | 0 | 50 | 50 | 50 |
| 2.5 | 0.25 | 0 | 20 | 20 | 20 |
| 5 | 1 | 0 | 90 | 90 | 100* |
| 2.5 | 0.5 | 0 | 50 | 50 | 50 |
| 1.25 | 0.25 | 0 | 20 | 20 | 20 |
| 2.5 | 1 | 0 | 90 | 90 | 90 |
| 1.25 | 0.5 | 0 | 50 | 50 | 50 |
| 0.625 | 0.25 | 0 | 20 | 20 | 20 |

TABLE 27

| | | AVERAGE | | | ACTUAL |
|---|---|---|---|---|---|
| PPM AI | | DEAD IN % | | EXPECTED | CON- |
| A6 | Difenoconazole | A6 | Difenoconazole | CONTROL | TROL |
| 20 | 0.5 | 0 | 50 | 50 | 70* |
| 10 | 0.25 | 0 | 20 | 20 | 20 |
| 10 | 0.5 | 0 | 50 | 50 | 50 |
| 5 | 0.25 | 0 | 20 | 20 | 20 |
| 5 | 0.5 | 0 | 50 | 50 | 70* |
| 2.5 | 0.25 | 0 | 20 | 20 | 20 |
| 2.5 | 0.5 | 0 | 50 | 50 | 70* |
| 1.25 | 0.25 | 0 | 20 | 20 | 20 |
| 1.25 | 0.5 | 0 | 50 | 50 | 50 |
| 0.625 | 0.25 | 0 | 20 | 20 | 20 |

TABLE 28

| PPM AI | | AVERAGE | | | |
|---|---|---|---|---|---|
| | COM- | DEAD IN % | | | |
| | POUND | | COMPOUND | EXPECTED | ACTUAL |
| A6 | III | A6 | III | CONTROL | CONTROL |
| 0.625 | 0.015625 | 0 | 90 | 90 | 90 |
| 0.3125 | 0.0078125 | 0 | 20 | 20 | 20 |
| 0.3125 | 0.015625 | 0 | 90 | 90 | 90 |
| 0.15625 | 0.0078125 | 0 | 20 | 20 | 20 |
| 0.15625 | 0.015625 | 0 | 90 | 90 | 90 |
| 0.078125 | 0.0078125 | 0 | 20 | 20 | 20 |
| 0.078125 | 0.015625 | 0 | 90 | 90 | 90 |
| 0.0390625 | 0.0078125 | 0 | 20 | 20 | 50* |
| 0.0390625 | 0.015625 | 0 | 90 | 90 | 90 |

TABLE 29

| | | AVERAGE | | | ACTUAL |
|---|---|---|---|---|---|
| PPM AI | | DEAD IN % | | EXPECTED | CON- |
| A8 | Azoxystrobin | A8 | Azoxystrobin | CONTROL | TROL |
| 20 | 0.5 | 0 | 20 | 20 | 20 |
| 10 | 0.25 | 0 | 20 | 20 | 20 |
| 5 | 0.125 | 0 | 20 | 20 | 20 |
| 20 | 1 | 0 | 20 | 20 | 20 |
| 10 | 0.5 | 0 | 20 | 20 | 20 |
| 5 | 0.25 | 0 | 20 | 20 | 20 |
| 2.5 | 0.125 | 0 | 20 | 20 | 20 |
| 20 | 2 | 0 | 20 | 20 | 20 |
| 10 | 1 | 0 | 20 | 20 | 20 |
| 5 | 0.5 | 0 | 20 | 20 | 20 |
| 2.5 | 0.25 | 0 | 20 | 20 | 20 |
| 1.25 | 0.125 | 0 | 20 | 20 | 20 |
| 10 | 2 | 0 | 20 | 20 | 20 |
| 5 | 1 | 0 | 20 | 20 | 20 |
| 2.5 | 0.5 | 0 | 20 | 20 | 20 |
| 1.25 | 0.25 | 0 | 20 | 20 | 20 |
| 0.625 | 0.125 | 0 | 20 | 20 | 20 |
| 5 | 2 | 0 | 20 | 20 | 20 |
| 2.5 | 1 | 0 | 20 | 20 | 20 |
| 1.25 | 0.5 | 0 | 20 | 20 | 20 |
| 0.625 | 0.25 | 0 | 20 | 20 | 20 |
| 0.3125 | 0.125 | 0 | 20 | 20 | 0 |

TABLE 30

| | | AVERAGE | | | |
|---|---|---|---|---|---|
| PPM AI | | DEAD IN % | | EXPECTED | ACTUAL |
| A8 | Isopyrazam | A8 | Isopyrazam | CONTROL | CONTROL |
| 5 | 0.125 | 0 | 100 | 100 | 90 |
| 2.5 | 0.0625 | 0 | 70 | 70 | 70 |
| 1.25 | 0.03125 | 0 | 20 | 20 | 20 |
| 2.5 | 0.125 | 0 | 100 | 100 | 90 |
| 1.25 | 0.0625 | 0 | 70 | 70 | 70 |
| 0.625 | 0.03125 | 0 | 20 | 20 | 20 |
| 1.25 | 0.125 | 0 | 100 | 100 | 90 |
| 0.625 | 0.0625 | 0 | 70 | 70 | 70 |
| 0.3125 | 0.03125 | 0 | 20 | 20 | 50* |
| 0.625 | 0.125 | 0 | 100 | 100 | 90 |
| 0.3125 | 0.0625 | 0 | 70 | 70 | 70 |
| 0.15625 | 0.03125 | 0 | 20 | 20 | 50* |
| 0.15625 | 0.0625 | 0 | 70 | 70 | 70 |
| 0.078125 | 0.03125 | 0 | 20 | 20 | 20 |
| 0.0390625 | 0.015625 | 0 | 0 | 0 | 0 |

TABLE 31

| | | AVERAGE | | | ACTUAL |
|---|---|---|---|---|---|
| PPM AI | | DEAD IN % | | EXPECTED | CON- |
| A8 | Cyproconazole | A8 | Cyproconazole | CONTROL | TROL |
| 20 | 0.5 | 0 | 50 | 50 | 50 |
| 10 | 0.25 | 0 | 20 | 20 | 20 |
| 20 | 1 | 0 | 90 | 90 | 90 |
| 10 | 0.5 | 0 | 50 | 50 | 50 |
| 5 | 0.25 | 0 | 20 | 20 | 0 |
| 10 | 1 | 0 | 90 | 90 | 100* |
| 5 | 0.5 | 0 | 50 | 50 | 50 |
| 2.5 | 0.25 | 0 | 20 | 20 | 20 |
| 5 | 1 | 0 | 90 | 90 | 100* |
| 2.5 | 0.5 | 0 | 50 | 50 | 50 |
| 1.25 | 0.25 | 0 | 20 | 20 | 20 |
| 5 | 2 | 0 | 100 | 100 | 100 |
| 2.5 | 1 | 0 | 90 | 90 | 100* |
| 1.25 | 0.5 | 0 | 50 | 50 | 50 |
| 0.625 | 0.25 | 0 | 20 | 20 | 20 |

TABLE 32

| | | AVERAGE | | | ACTUAL |
|---|---|---|---|---|---|
| PPM AI | | DEAD IN % | | EXPECTED | CON- |
| A8 | Difenoconazole | A8 | Difenoconazole | CONTROL | TROL |
| 20 | 0.5 | 0 | 70 | 70 | 50 |
| 10 | 0.25 | 0 | 20 | 20 | 20 |
| 20 | 1 | 0 | 100 | 100 | 90 |
| 10 | 0.5 | 0 | 70 | 70 | 70 |
| 5 | 0.25 | 0 | 20 | 20 | 20 |
| 20 | 2 | 0 | 100 | 100 | 100 |
| 10 | 1 | 0 | 100 | 100 | 100 |
| 5 | 0.5 | 0 | 70 | 70 | 50 |
| 2.5 | 0.25 | 0 | 20 | 20 | 20 |
| 10 | 2 | 0 | 100 | 100 | 100 |
| 5 | 1 | 0 | 100 | 100 | 90 |
| 2.5 | 0.5 | 0 | 70 | 70 | 70 |
| 1.25 | 0.25 | 0 | 20 | 20 | 20 |
| 5 | 2 | 0 | 100 | 100 | 100 |
| 2.5 | 1 | 0 | 100 | 100 | 100 |
| 1.25 | 0.5 | 0 | 70 | 70 | 70 |
| 0.625 | 0.25 | 0 | 20 | 20 | 20 |

TABLE 33

| PPM AI | | AVERAGE | | | |
|---|---|---|---|---|---|
| | COM- | DEAD IN % | | | |
| | POUND | | COMPOUND | EXPECTED | ACTUAL |
| A8 | III | A8 | III | CONTROL | CONTROL |
| 0.625 | 0.015625 | 0 | 70 | 70 | 90* |
| 0.3125 | 0.0078125 | 0 | 50 | 50 | 70* |
| 0.3125 | 0.015625 | 0 | 70 | 70 | 70 |
| 0.15625 | 0.0078125 | 0 | 50 | 50 | 50 |
| 0.15625 | 0.015625 | 0 | 70 | 70 | 90* |
| 0.078125 | 0.0078125 | 0 | 50 | 50 | 0 |
| 0.078125 | 0.015625 | 0 | 70 | 70 | 90* |
| 0.0390625 | 0.0078125 | 0 | 50 | 50 | 70* |
| 0.0390625 | 0.015625 | 0 | 70 | 70 | 90* |

Gaeumannomyces graminis:

Mycelial fragments of the fungus from cryogenic storage were directly mixed into nutrient broth (potato dextrose broth). After placing a (DMSO) solution of the test compounds into a microliter plate (96-well format) the nutrient broth containing the fungal spores was added. The test plates were incubated at 24° C. and the inhibition of growth was determined photometrically after 48 hours at 620 nm Application rates are shown in the Tables.

TABLE 34

| | | AVERAGE | | | |
|---|---|---|---|---|---|
| | | DEAD IN % | | | |
| PPM AI | | | Azoxy- | EXPECTED | ACTUAL |
| A1 | Azoxystrobin | A1 | strobin | CONTROL | CONTROL |
| 20 | 0.5 | 0 | 50 | 50 | 90* |
| 10 | 0.25 | 0 | 20 | 20 | 70* |
| 5 | 0.125 | 0 | 20 | 20 | 50* |
| 2.5 | 0.0625 | 0 | 0 | 0 | 20* |
| 1.25 | 0.03125 | 0 | 0 | 0 | 20* |
| 0.625 | 0.015625 | 0 | 0 | 0 | 20* |
| 0.3125 | 0.0078125 | 0 | 0 | 0 | 20* |
| 0.15625 | 0.00390625 | 0 | 0 | 0 | 20* |
| 20 | 1 | 0 | 50 | 50 | 50 |
| 10 | 0.5 | 0 | 50 | 50 | 20 |
| 5 | 0.25 | 0 | 20 | 20 | 20 |
| 2.5 | 0.125 | 0 | 20 | 20 | 20 |
| 1.25 | 0.0625 | 0 | 0 | 0 | 20* |
| 0.625 | 0.03125 | 0 | 0 | 0 | 20* |
| 0.3125 | 0.015625 | 0 | 0 | 0 | 20* |
| 0.15625 | 0.0078125 | 0 | 0 | 0 | 20* |
| 0.078125 | 0.00390625 | 0 | 0 | 0 | 20* |
| 20 | 2 | 0 | 50 | 50 | 50 |
| 10 | 1 | 0 | 50 | 50 | 20 |
| 5 | 0.5 | 0 | 50 | 50 | 0 |
| 2.5 | 0.25 | 0 | 20 | 20 | 0 |
| 1.25 | 0.125 | 0 | 20 | 20 | 0 |
| 10 | 2 | 0 | 50 | 50 | 20 |
| 5 | 1 | 0 | 50 | 50 | 20 |
| 2.5 | 0.5 | 0 | 50 | 50 | 20 |
| 1.25 | 0.25 | 0 | 20 | 20 | 20 |
| 0.625 | 0.125 | 0 | 20 | 20 | 20 |
| 0.3125 | 0.0625 | 0 | 0 | 0 | 20* |
| 0.15625 | 0.03125 | 0 | 0 | 0 | 20* |
| 0.078125 | 0.015625 | 0 | 0 | 0 | 20* |
| 0.0390625 | 0.0078125 | 0 | 0 | 0 | 20* |
| 5 | 2 | 0 | 50 | 50 | 50 |
| 2.5 | 1 | 0 | 50 | 50 | 50 |
| 1.25 | 0.5 | 0 | 50 | 50 | 20 |
| 0.625 | 0.25 | 0 | 20 | 20 | 20 |
| 0.3125 | 0.125 | 0 | 20 | 20 | 20 |
| 0.15625 | 0.0625 | 0 | 0 | 0 | 20* |
| 0.078125 | 0.03125 | 0 | 0 | 0 | 20* |
| 0.0390625 | 0.015625 | 0 | 0 | 0 | 20* |

TABLE 35

| PPM AI | | AVERAGE | | | |
|---|---|---|---|---|---|
| | Iso- | DEAD IN % | | EXPECTED | ACTUAL |
| A1 | pyrazam | A1 | Isopyrazam | CONTROL | CONTROL |
| 20 | 0.5 | 0 | 0 | 0 | 50* |
| 10 | 0.25 | 0 | 0 | 0 | 20* |

TABLE 36

| PPM AI | | AVERAGE | | | |
|---|---|---|---|---|---|
| | Chloro- | DEAD IN % | | EXPECTED | ACTUAL |
| A1 | thalonil | A1 | Chlorothalonil | CONTROL | CONTROL |
| 20 | 0.5 | 0 | 20 | 20 | 50* |
| 10 | 0.25 | 0 | 0 | 0 | 20* |
| 20 | 1 | 0 | 50 | 50 | 50 |
| 10 | 0.5 | 0 | 20 | 20 | 20 |
| 20 | 2 | 0 | 90 | 90 | 70 |
| 10 | 1 | 0 | 50 | 50 | 50 |
| 5 | 0.5 | 0 | 20 | 20 | 20 |
| 10 | 2 | 0 | 90 | 90 | 100* |
| 5 | 1 | 0 | 50 | 50 | 50 |
| 2.5 | 0.5 | 0 | 20 | 20 | 20 |
| 5 | 2 | 0 | 90 | 90 | 100* |
| 2.5 | 1 | 0 | 50 | 50 | 50 |
| 1.25 | 0.5 | 0 | 20 | 20 | 20 |

TABLE 37

| | | AVERAGE | | | |
|---|---|---|---|---|---|
| PPM AI | | DEAD IN % | | EXPECTED | ACTUAL |
| A1 | Cyproconazole | A1 | Cyproconazole | CONTROL | CONTROL |
| 20 | 0.5 | 0 | 50 | 50 | 50 |
| 10 | 0.25 | 0 | 20 | 20 | 20 |
| 20 | 1 | 0 | 70 | 70 | 90* |
| 10 | 0.5 | 0 | 50 | 50 | 50 |
| 5 | 0.25 | 0 | 20 | 20 | 20 |
| 20 | 2 | 0 | 90 | 90 | 90 |
| 10 | 1 | 0 | 70 | 70 | 90* |
| 5 | 0.5 | 0 | 50 | 50 | 50 |
| 2.5 | 0.25 | 0 | 20 | 20 | 20 |
| 10 | 2 | 0 | 90 | 90 | 90 |
| 5 | 1 | 0 | 70 | 70 | 90* |
| 2.5 | 0.5 | 0 | 50 | 50 | 50 |
| 5 | 2 | 0 | 90 | 90 | 90 |
| 2.5 | 1 | 0 | 70 | 70 | 70 |
| 1.25 | 0.5 | 0 | 50 | 50 | 20 |

TABLE 38

| | | AVERAGE | | | |
|---|---|---|---|---|---|
| PPM AI | | DEAD IN % | | EXPECTED | ACTUAL |
| A1 | Difenoconazole | A1 | Difenoconazole | CONTROL | CONTROL |
| 20 | 0.5 | 0 | 0 | 0 | 50* |
| 10 | 0.25 | 0 | 0 | 0 | 20* |

TABLE 39

| | | AVERAGE | | | ACTUAL |
|---|---|---|---|---|---|
| PPM AI | | DEAD IN % | | EXPECTED | CON- |
| A1 | Mandipropamid | A1 | Mandipropamid | CONTROL | TROL |
| 20 | 0.5 | 0 | 0 | 0 | 50* |
| 10 | 0.25 | 0 | 0 | 0 | 20* |

TABLE 40

| | | AVERAGE | | | |
|---|---|---|---|---|---|
| PPM AI | | DEAD IN % | | EXPECTED | ACTUAL |
| A1 | Mefenoxam | A1 | Mefenoxam | CONTROL | CONTROL |
| 20 | 0.5 | 0 | 0 | 0 | 50* |
| 10 | 0.25 | 0 | 0 | 0 | 20* |

TABLE 41

| | | AVERAGE | | | |
|---|---|---|---|---|---|
| PPM AI | | DEAD IN % | | | ACTUAL |
| | COM- | | COM- | EXPECTED | CON- |
| A1 | POUND III | A1 | POUND III | CONTROL | TROL |
| 20 | 0.5 | 0 | 100 | 100 | 70 |
| 10 | 0.25 | 0 | 100 | 100 | 70 |
| 5 | 0.125 | 0 | 90 | 90 | 50 |
| 2.5 | 0.0625 | 0 | 70 | 70 | 50 |
| 1.25 | 0.03125 | 0 | 20 | 20 | 20 |
| 0.625 | 0.015625 | 0 | 20 | 20 | 20 |
| 20 | 1 | 0 | 100 | 100 | 70 |
| 10 | 0.5 | 0 | 100 | 100 | 70 |
| 5 | 0.25 | 0 | 100 | 100 | 70 |
| 2.5 | 0.125 | 0 | 90 | 90 | 70 |
| 1.25 | 0.0625 | 0 | 70 | 70 | 70 |
| 0.625 | 0.03125 | 0 | 20 | 20 | 20 |
| 0.3125 | 0.015625 | 0 | 20 | 20 | 0 |
| 20 | 2 | 0 | 100 | 100 | 90 |
| 10 | 1 | 0 | 100 | 100 | 90 |
| 5 | 0.5 | 0 | 100 | 100 | 90 |
| 2.5 | 0.25 | 0 | 100 | 100 | 90 |
| 1.25 | 0.125 | 0 | 90 | 90 | 90 |
| 0.625 | 0.0625 | 0 | 70 | 70 | 90* |
| 0.3125 | 0.03125 | 0 | 20 | 20 | 70* |
| 0.15625 | 0.015625 | 0 | 20 | 20 | 50* |
| 0.078125 | 0.0078125 | 0 | 0 | 0 | 20* |
| 0.0390625 | 0.00390625 | 0 | 0 | 0 | 20* |
| 5 | 1 | 0 | 100 | 100 | 90 |
| 2.5 | 0.5 | 0 | 100 | 100 | 90 |
| 1.25 | 0.25 | 0 | 100 | 100 | 90 |
| 0.625 | 0.125 | 0 | 90 | 90 | 90 |
| 0.3125 | 0.0625 | 0 | 70 | 70 | 90* |
| 0.15625 | 0.03125 | 0 | 20 | 20 | 90* |
| 0.078125 | 0.015625 | 0 | 20 | 20 | 90* |
| 0.0390625 | 0.0078125 | 0 | 0 | 0 | 90* |
| 0.3125 | 0.125 | 0 | 90 | 90 | 100* |
| 0.15625 | 0.0625 | 0 | 70 | 70 | 100* |
| 0.078125 | 0.03125 | 0 | 20 | 20 | 100* |
| 0.0390625 | 0.015625 | 0 | 20 | 20 | 100* |

TABLE 42

| PPM AI | | AVERAGE | | | |
|---|---|---|---|---|---|
| | COM- | DEAD IN % | | | |
| | POUND | | COM- | EXPECTED | ACTUAL |
| A1 | IV | A1 | POUND IV | CONTROL | CONTROL |
| 20 | 0.5 | 0 | 0 | 0 | 50* |
| 10 | 0.25 | 0 | 0 | 0 | 20* |

TABLE 43

| PPM AI | | AVERAGE | | | |
|---|---|---|---|---|---|
| | Azoxy- | DEAD IN % | | EXPECTED | ACTUAL |
| A5 | strobin | A5 | Azoxystrobin | CONTROL | CONTROL |
| 1.25 | 0.03125 | 0 | 100 | 100 | 90 |
| 0.625 | 0.015625 | 0 | 90 | 90 | 90 |
| 0.3125 | 0.015625 | 0 | 90 | 90 | 100* |
| 0.15625 | 0.0078125 | 0 | 0 | 0 | 20* |
| 0.15625 | 0.015625 | 0 | 90 | 90 | 100* |
| 0.078125 | 0.0078125 | 0 | 0 | 0 | 20* |
| 0.078125 | 0.015625 | 0 | 90 | 90 | 100* |
| 0.0390625 | 0.0078125 | 0 | 0 | 0 | 20* |
| 0.0390625 | 0.015625 | 0 | 90 | 90 | 100* |

TABLE 44

| | | AVERAGE | | | |
|---|---|---|---|---|---|
| PPM AI | | DEAD IN % | | EXPECTED | ACTUAL |
| A5 | Isopyrazam | A5 | Isopyrazam | CONTROL | CONTROL |
| 20 | 0.5 | 0 | 0 | 0 | 20* |
| 20 | 1 | 0 | 0 | 0 | 50* |

TABLE 45

| | | AVERAGE | | | |
|---|---|---|---|---|---|
| PPM AI | | DEAD IN % | | EXPECTED | ACTUAL |
| A5 | Chlorothalonil | A5 | Chlorothalonil | CONTROL | CONTROL |
| 20 | 0.5 | 0 | 20 | 20 | 100* |
| 10 | 0.25 | 0 | 0 | 0 | 20* |
| 20 | 1 | 0 | 100 | 100 | 100 |
| 10 | 0.5 | 0 | 20 | 20 | 50* |
| 5 | 0.5 | 0 | 20 | 20 | 20 |
| 2.5 | 0.5 | 0 | 20 | 20 | 50* |
| 1.25 | 0.5 | 0 | 20 | 20 | 90* |

TABLE 46

| | | AVERAGE | | | ACTUAL |
|---|---|---|---|---|---|
| PPM AI | | DEAD IN % | | EXPECTED | CON- |
| A5 | Cyproconazole | A5 | Cyproconazole | CONTROL | TROL |
| 20 | 0.5 | 0 | 90 | 90 | 70 |
| 10 | 0.25 | 0 | 20 | 20 | 20 |
| 10 | 0.5 | 0 | 90 | 90 | 70 |
| 5 | 0.25 | 0 | 20 | 20 | 20 |
| 5 | 0.5 | 0 | 90 | 90 | 90 |
| 2.5 | 0.25 | 0 | 20 | 20 | 20 |
| 2.5 | 0.5 | 0 | 90 | 90 | 90 |
| 1.25 | 0.25 | 0 | 20 | 20 | 20 |
| 1.25 | 0.5 | 0 | 90 | 90 | 90 |
| 0.625 | 0.25 | 0 | 20 | 20 | 50* |

TABLE 47

| | | AVERAGE | | | |
|---|---|---|---|---|---|
| PPM AI | | DEAD IN % | | EXPECTED | ACTUAL |
| A5 | Difenoconazole | A5 | Difenoconazole | CONTROL | CONTROL |
| 20 | 0.5 | 0 | 0 | 0 | 20* |
| 20 | 1 | 0 | 20 | 20 | 90* |
| 20 | 2 | 0 | 90 | 90 | 90 |
| 10 | 1 | 0 | 20 | 20 | 20 |
| 10 | 2 | 0 | 90 | 90 | 90 |
| 5 | 1 | 0 | 20 | 20 | 50* |
| 5 | 2 | 0 | 90 | 90 | 70 |
| 2.5 | 1 | 0 | 20 | 20 | 50* |
| 1.25 | 0.5 | 0 | 0 | 0 | 20* |

TABLE 48

| PPM AI | | AVERAGE | | | |
|---|---|---|---|---|---|
| | Mandi- | DEAD IN % | | EXPECTED | ACTUAL |
| A5 | propamid | A5 | Mandipropamid | CONTROL | CONTROL |
| 20 | 0.5 | 0 | 0 | 0 | 20* |
| 20 | 1 | 0 | 0 | 0 | 20* |

TABLE 49

| | | AVERAGE DEAD | | | |
|---|---|---|---|---|---|
| PPM AI | | IN % | | EXPECTED | ACTUAL |
| A5 | Mefenoxam | A5 | Mefenoxam | CONTROL | CONTROL |
| 20 | 0.5 | 0 | 0 | 0 | 20* |
| 20 | 1 | 0 | 0 | 0 | 20* |

TABLE 50

| | | AVERAGE | | | |
|---|---|---|---|---|---|
| | | DEAD IN % | | | |
| PPM AI | | | COM- | | |
| | COMPOUND | | POUND | EXPECTED | ACTUAL |
| A5 | III | A5 | III | CONTROL | CONTROL |
| 5 | 0.125 | 0 | 90 | 90 | 90 |
| 2.5 | 0.0625 | 0 | 90 | 90 | 50 |
| 1.25 | 0.03125 | 0 | 50 | 50 | 20 |
| 2.5 | 0.125 | 0 | 90 | 90 | 100* |
| 1.25 | 0.0625 | 0 | 90 | 90 | 70 |
| 0.625 | 0.03125 | 0 | 50 | 50 | 20 |
| 0.3125 | 0.015625 | 0 | 0 | 0 | 0 |
| 0.15625 | 0.0078125 | 0 | 0 | 0 | 0 |
| 0.078125 | 0.00390625 | 0 | 0 | 0 | 0 |
| 1.25 | 0.125 | 0 | 90 | 90 | 100* |
| 0.625 | 0.0625 | 0 | 90 | 90 | 90 |
| 0.3125 | 0.03125 | 0 | 50 | 50 | 20 |
| 0.625 | 0.125 | 0 | 90 | 90 | 90 |
| 0.3125 | 0.0625 | 0 | 90 | 90 | 90 |
| 0.15625 | 0.03125 | 0 | 50 | 50 | 50 |
| 0.3125 | 0.125 | 0 | 90 | 90 | 100* |
| 0.15625 | 0.0625 | 0 | 90 | 90 | 100* |
| 0.078125 | 0.03125 | 0 | 50 | 50 | 50 |
| 0.0390625 | 0.015625 | 0 | 0 | 0 | 0 |

TABLE 51

| PPM AI | | AVERAGE | | | |
|---|---|---|---|---|---|
| | COM- | DEAD IN % | | | |
| | POUND | | COMPOUND | EXPECTED | ACTUAL |
| A5 | IV | A5 | IV | CONTROL | CONTROL |
| 20 | 0.5 | 0 | 0 | 0 | 20* |
| 20 | 1 | 0 | 0 | 0 | 20* |
| 10 | 0.5 | 0 | 0 | 0 | 20* |

TABLE 52

| | | | | EXPECT- | AC- |
|---|---|---|---|---|---|
| | | AVERAGE DEAD | | ED | TUAL |
| PPM AI | | IN % | | CON- | CON- |
| A6 | Azoxystrobin | A6 | Azoxystrobin | TROL | TROL |
| 20 | 0.5 | 0 | 90 | 90 | 90 |
| 10 | 0.25 | 0 | 70 | 70 | 70 |
| 5 | 0.125 | 0 | 70 | 70 | 50 |
| 2.5 | 0.0625 | 0 | 50 | 50 | 20 |
| 1.25 | 0.03125 | 0 | 50 | 50 | 20 |
| 0.625 | 0.015625 | 0 | 50 | 50 | 20 |
| 0.3125 | 0.0078125 | 0 | 20 | 20 | 20 |
| 0.15625 | 0.00390625 | 0 | 20 | 20 | 20 |
| 20 | 1 | 0 | 90 | 90 | 90 |
| 10 | 0.5 | 0 | 90 | 90 | 90 |
| 5 | 0.25 | 0 | 70 | 70 | 70 |
| 2.5 | 0.125 | 0 | 70 | 70 | 50 |
| 1.25 | 0.0625 | 0 | 50 | 50 | 50 |
| 0.625 | 0.03125 | 0 | 50 | 50 | 50 |
| 0.3125 | 0.015625 | 0 | 50 | 50 | 50 |
| 0.15625 | 0.0078125 | 0 | 20 | 20 | 20 |
| 0.078125 | 0.00390625 | 0 | 20 | 20 | 20 |
| 20 | 2 | 0 | 90 | 90 | 90 |
| 10 | 1 | 0 | 90 | 90 | 90 |
| 5 | 0.5 | 0 | 90 | 90 | 90 |
| 2.5 | 0.25 | 0 | 70 | 70 | 50 |
| 1.25 | 0.125 | 0 | 70 | 70 | 50 |
| 0.625 | 0.0625 | 0 | 50 | 50 | 50 |
| 0.3125 | 0.03125 | 0 | 50 | 50 | 50 |
| 0.15625 | 0.015625 | 0 | 50 | 50 | 50 |
| 0.078125 | 0.0078125 | 0 | 20 | 20 | 20 |
| 0.0390625 | 0.00390625 | 0 | 20 | 20 | 20 |
| 10 | 2 | 0 | 90 | 90 | 90 |
| 5 | 1 | 0 | 90 | 90 | 90 |
| 2.5 | 0.5 | 0 | 90 | 90 | 70 |
| 1.25 | 0.25 | 0 | 70 | 70 | 70 |
| 0.625 | 0.125 | 0 | 70 | 70 | 50 |
| 0.3125 | 0.0625 | 0 | 50 | 50 | 50 |
| 0.15625 | 0.03125 | 0 | 50 | 50 | 50 |
| 0.078125 | 0.015625 | 0 | 50 | 50 | 50 |
| 0.0390625 | 0.0078125 | 0 | 20 | 20 | 50* |
| 5 | 2 | 0 | 90 | 90 | 90 |
| 2.5 | 1 | 0 | 90 | 90 | 70 |
| 1.25 | 0.5 | 0 | 90 | 90 | 70 |
| 0.625 | 0.25 | 0 | 70 | 70 | 70 |
| 0.3125 | 0.125 | 0 | 70 | 70 | 70 |
| 0.15625 | 0.0625 | 0 | 50 | 50 | 50 |
| 0.078125 | 0.03125 | 0 | 50 | 50 | 50 |
| 0.0390625 | 0.015625 | 0 | 50 | 50 | 50 |

TABLE 53

| | | AVERAGE | | EX- | |
|---|---|---|---|---|---|
| PPM AI | | DEAD IN %, | | PECTED | ACTUAL |
| A6 | Isopyrazam | A6 | Isopyrazam | CONTROL | CONTROL |
| 20 | 1 | 0 | 20 | 20 | 20 |
| 20 | 2 | 0 | 50 | 50 | 20 |
| 10 | 1 | 0 | 20 | 20 | 20 |
| 10 | 2 | 0 | 50 | 50 | 20 |
| 5 | 1 | 0 | 20 | 20 | 0 |
| 5 | 2 | 0 | 50 | 50 | 50 |
| 2.5 | 1 | 0 | 20 | 20 | 0 |

TABLE 54

| | | AVERAGE DEAD | | | |
|---|---|---|---|---|---|
| PPM AI | | IN % | | EXPECTED | ACTUAL |
| A6 | Chlorothalonil | A6 | Chlorothalonil | CONTROL | CONTROL |
| 20 | 0.5 | 0 | 50 | 50 | 70* |
| 10 | 0.25 | 0 | 0 | 0 | 20* |
| 20 | 1 | 0 | 90 | 90 | 100* |
| 10 | 0.5 | 0 | 50 | 50 | 70* |
| 10 | 1 | 0 | 90 | 90 | 90 |
| 5 | 0.5 | 0 | 50 | 50 | 20 |
| 5 | 1 | 0 | 90 | 90 | 100* |
| 2.5 | 0.5 | 0 | 50 | 50 | 70* |
| 5 | 2 | 0 | 100 | 100 | 100 |
| 2.5 | 1 | 0 | 90 | 90 | 90 |
| 1.25 | 0.5 | 0 | 50 | 50 | 50 |

TABLE 55

| | | AVERAGE DEAD | | | |
|---|---|---|---|---|---|
| PPM AI | | IN % | | EXPECTED | ACTUAL |
| A6 | Cyproconazole | A6 | Cyproconazole | CONTROL | CONTROL |
| 20 | 0.5 | 0 | 50 | 50 | 50 |
| 20 | 1 | 0 | 70 | 70 | 70 |
| 10 | 0.5 | 0 | 50 | 50 | 50 |
| 20 | 2 | 0 | 90 | 90 | 90 |
| 10 | 1 | 0 | 70 | 70 | 70 |
| 5 | 0.5 | 0 | 50 | 50 | 50 |
| 10 | 2 | 0 | 90 | 90 | 90 |
| 5 | 1 | 0 | 70 | 70 | 70 |
| 2.5 | 0.5 | 0 | 50 | 50 | 50 |
| 5 | 2 | 0 | 90 | 90 | 90 |
| 2.5 | 1 | 0 | 70 | 70 | 70 |
| 1.25 | 0.5 | 0 | 50 | 50 | 50 |

TABLE 56

| | | AVERAGE DEAD | | EX- | ACTUAL |
|---|---|---|---|---|---|
| PPM AI | | IN % | | PECTED | CON- |
| A6 | Mandipropamid | A6 | Mandipropamid | CONTROL | TROL |
| 20 | 1 | 0 | 0 | 0 | 70* |

TABLE 57

| | | AVERAGE | | | |
|---|---|---|---|---|---|
| PPM AI | | DEAD IN % | | EX- | |
| | COM- | | COM- | PECTED | ACTUAL |
| A6 | POUND III | A6 | POUND III | CONTROL | CONTROL |
| 2.5 | 0.0625 | 0 | 90 | 90 | 90 |
| 1.25 | 0.03125 | 0 | 50 | 50 | 50 |
| 1.25 | 0.0625 | 0 | 90 | 90 | 90 |
| 0.625 | 0.03125 | 0 | 50 | 50 | 50 |
| 0.625 | 0.0625 | 0 | 90 | 90 | 90 |
| 0.3125 | 0.03125 | 0 | 50 | 50 | 50 |
| 0.3125 | 0.0625 | 0 | 90 | 90 | 90 |
| 0.15625 | 0.03125 | 0 | 50 | 50 | 70* |
| 0.15625 | 0.0625 | 0 | 90 | 90 | 90 |
| 0.078125 | 0.03125 | 0 | 50 | 50 | 70* |
| 0.0390625 | 0.015625 | 0 | 0 | 0 | 20* |

TABLE 58

| PPM AI | | AVERAGE DEAD | | | |
|---|---|---|---|---|---|
| | COM- | IN % | | | |
| | POUND | | COMPOUND | EXPECTED | ACTUAL |
| A6 | IV | A6 | IV | CONTROL | CONTROL |
| 20 | 1 | 0 | 0 | 0 | 70* |

TABLE 59

| | | | | EXPECT- | AC- |
|---|---|---|---|---|---|
| | | AVERAGE DEAD | | ED | TUAL |
| PPM AI | | IN % | | CON- | CON- |
| A8 | Azoxystrobin | A8 | Azoxystrobin | TROL | TROL |
| 20 | 0.5 | 0 | 70 | 70 | 70 |
| 10 | 0.25 | 0 | 50 | 50 | 70* |
| 5 | 0.125 | 0 | 50 | 50 | 70* |
| 2.5 | 0.0625 | 0 | 50 | 50 | 50 |
| 1.25 | 0.03125 | 0 | 50 | 50 | 50 |
| 0.625 | 0.015625 | 0 | 50 | 50 | 50 |
| 0.3125 | 0.0078125 | 0 | 50 | 50 | 20 |
| 0.15625 | 0.00390625 | 0 | 20 | 20 | 0 |
| 20 | 1 | 0 | 70 | 70 | 90* |
| 10 | 0.5 | 0 | 70 | 70 | 70 |
| 5 | 0.25 | 0 | 50 | 50 | 70* |
| 2.5 | 0.125 | 0 | 50 | 50 | 70* |
| 1.25 | 0.0625 | 0 | 50 | 50 | 50 |
| 0.625 | 0.03125 | 0 | 50 | 50 | 50 |
| 0.3125 | 0.015625 | 0 | 50 | 50 | 50 |
| 0.15625 | 0.0078125 | 0 | 50 | 50 | 20 |
| 0.078125 | 0.00390625 | 0 | 20 | 20 | 0 |
| 20 | 2 | 0 | 90 | 90 | 90 |
| 10 | 1 | 0 | 70 | 70 | 70 |
| 5 | 0.5 | 0 | 70 | 70 | 70 |
| 2.5 | 0.25 | 0 | 50 | 50 | 70* |
| 1.25 | 0.125 | 0 | 50 | 50 | 70* |
| 0.625 | 0.0625 | 0 | 50 | 50 | 50 |
| 0.3125 | 0.03125 | 0 | 50 | 50 | 50 |
| 0.15625 | 0.015625 | 0 | 50 | 50 | 50 |
| 0.078125 | 0.0078125 | 0 | 50 | 50 | 20 |
| 0.0390625 | 0.00390625 | 0 | 20 | 20 | 0 |
| 10 | 2 | 0 | 90 | 90 | 90 |
| 5 | 1 | 0 | 70 | 70 | 70 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 50 | 50 | 50 |
| 0.15625 | 0.03125 | 0 | 50 | 50 | 50 |
| 0.078125 | 0.015625 | 0 | 50 | 50 | 50 |
| 0.0390625 | 0.0078125 | 0 | 50 | 50 | 20 |
| 5 | 2 | 0 | 90 | 90 | 90 |
| 2.5 | 1 | 0 | 70 | 70 | 70 |
| 1.25 | 0.5 | 0 | 70 | 70 | 70 |
| 0.625 | 0.25 | 0 | 50 | 50 | 70* |
| 0.3125 | 0.125 | 0 | 50 | 50 | 50 |
| 0.15625 | 0.0625 | 0 | 50 | 50 | 50 |
| 0.078125 | 0.03125 | 0 | 50 | 50 | 50 |
| 0.0390625 | 0.015625 | 0 | 50 | 50 | 50 |

TABLE 60

| | | AVERAGE DEAD | | | |
|---|---|---|---|---|---|
| PPM AI | | IN % | | EXPECTED | ACTUAL |
| A8 | Chlorothalonil | A8 | Chlorothalonil | CONTROL | CONTROL |
| 20 | 1 | 0 | 70 | 70 | 70 |
| 10 | 1 | 0 | 70 | 70 | 70 |
| 5 | 1 | 0 | 70 | 70 | 70 |
| 2.5 | 0.5 | 0 | 0 | 0 | 20* |
| 2.5 | 1 | 0 | 70 | 70 | 70 |
| 1.25 | 0.5 | 0 | 0 | 0 | 20* |

TABLE 61

| | | AVERAGE DEAD | | | |
|---|---|---|---|---|---|
| PPM AI | | IN % | | EXPECTED | ACTUAL |
| A8 | Cyproconazole | A8 | Cyproconazole | CONTROL | CONTROL |
| 20 | 0.5 | 0 | 50 | 50 | 50 |
| 20 | 1 | 0 | 90 | 90 | 90 |
| 10 | 0.5 | 0 | 50 | 50 | 50 |
| 20 | 2 | 0 | 90 | 90 | 90 |
| 10 | 1 | 0 | 90 | 90 | 90 |
| 5 | 0.5 | 0 | 50 | 50 | 50 |
| 10 | 2 | 0 | 90 | 90 | 90 |
| 5 | 1 | 0 | 90 | 90 | 90 |
| 2.5 | 0.5 | 0 | 50 | 50 | 50 |
| 5 | 2 | 0 | 90 | 90 | 90 |
| 2.5 | 1 | 0 | 90 | 90 | 90 |
| 1.25 | 0.5 | 0 | 50 | 50 | 50 |

TABLE 62

| | | AVERAGE | | | |
|---|---|---|---|---|---|
| PPM AI | | DEAD IN % | | | |
| | COM- | | COM- | EXPECTED | ACTUAL |
| A8 | POUND III | A8 | POUND III | CONTROL | CONTROL |
| 2.5 | 0.0625 | 0 | 90 | 90 | 90 |
| 1.25 | 0.03125 | 0 | 70 | 70 | 50 |
| 0.625 | 0.015625 | 0 | 20 | 20 | 20 |
| 1.25 | 0.0625 | 0 | 90 | 90 | 90 |
| 0.625 | 0.03125 | 0 | 70 | 70 | 50 |
| 0.3125 | 0.015625 | 0 | 20 | 20 | 0 |
| 0.625 | 0.0625 | 0 | 90 | 90 | 90 |
| 0.3125 | 0.03125 | 0 | 70 | 70 | 50 |
| 0.15625 | 0.015625 | 0 | 20 | 20 | 0 |
| 0.3125 | 0.0625 | 0 | 90 | 90 | 90 |
| 0.15625 | 0.03125 | 0 | 70 | 70 | 50 |
| 0.078125 | 0.015625 | 0 | 20 | 20 | 20 |
| 0.15625 | 0.0625 | 0 | 90 | 90 | 100* |
| 0.078125 | 0.03125 | 0 | 70 | 70 | 70 |
| 0.0390625 | 0.015625 | 0 | 20 | 20 | 50* |

Monographella nivalis (syn. Microdochium nivale, Fusarium nivale), Snow Mould, Foot Rot:

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours at 620 nm. Application rates are shown in the Tables.

TABLE 63

| | | | | EXPECT- | OB- |
|---|---|---|---|---|---|
| | | AVERAGE DEAD | | ED | SERVED |
| PPM AI | | IN % | | CON- | CON- |
| A1 | Azoxystrobin | A1 | Azoxystrobin | TROL | TROL |
| 1.25 | 0.03125 | 0 | 90 | 90 | 100* |
| 0.625 | 0.015625 | 0 | 50 | 50 | 70* |
| 0.3125 | 0.0078125 | 0 | 20 | 20 | 20 |
| 0.625 | 0.03125 | 0 | 90 | 90 | 100* |
| 0.3125 | 0.015625 | 0 | 50 | 50 | 50 |
| 0.15625 | 0.0078125 | 0 | 20 | 20 | 20 |
| 0.3125 | 0.03125 | 0 | 90 | 90 | 100* |
| 0.15625 | 0.015625 | 0 | 50 | 50 | 50 |
| 0.078125 | 0.0078125 | 0 | 20 | 20 | 20 |
| 0.15625 | 0.03125 | 0 | 90 | 90 | 100* |
| 0.078125 | 0.015625 | 0 | 50 | 50 | 90* |
| 0.0390625 | 0.0078125 | 0 | 20 | 20 | 50* |
| 0.078125 | 0.03125 | 0 | 90 | 90 | 100* |
| 0.0390625 | 0.015625 | 0 | 50 | 50 | 100* |

TABLE 64

| PPM AI | | AVERAGE | | | |
|---|---|---|---|---|---|
| | Iso- | DEAD IN % | | EXPECTED | OBSERVED |
| A1 | pyrazam | A1 | Isopyrazam | CONTROL | CONTROL |
| 20 | 0.5 | 0 | 0 | 0 | 20* |
| 10 | 0.25 | 0 | 0 | 0 | 20* |

TABLE 65

| PPM AI | | AVERAGE | | | |
|---|---|---|---|---|---|
| | Chlo- | DEAD IN % | | EXPECTED | OBSERVED |
| A1 | rothalonil | A1 | Chlorothalonil | CONTROL | CONTROL |
| 20 | 0.5 | 0 | 70 | 70 | 100* |
| 10 | 0.25 | 0 | 0 | 0 | 50* |
| 10 | 0.5 | 0 | 70 | 70 | 100* |
| 5 | 0.5 | 0 | 70 | 70 | 100* |
| 2.5 | 0.5 | 0 | 70 | 70 | 100* |
| 1.25 | 0.5 | 0 | 70 | 70 | 100** |

TABLE 66

| | | AVERAGE | | | |
|---|---|---|---|---|---|
| PPM AI | | DEAD IN % | | EXPECTED | OBSERVED |
| A1 | Difenoconazole | A1 | Difenoconazole | CONTROL | CONTROL |
| 20 | 0.5 | 0 | 0 | 0 | 20* |
| 20 | 1 | 0 | 20 | 20 | 0 |
| 20 | 2 | 0 | 100 | 100 | 90 |
| 10 | 1 | 0 | 20 | 20 | 0 |
| 10 | 2 | 0 | 100 | 100 | 50 |
| 5 | 1 | 0 | 20 | 20 | 0 |
| 5 | 2 | 0 | 100 | 100 | 100 |
| 2.5 | 1 | 0 | 20 | 20 | 20 |

TABLE 67

| PPM AI | | AVERAGE | | | |
|---|---|---|---|---|---|
| | Mandi- | DEAD IN % | | EXPECTED | OBSERVED |
| A1 | propamid | A1 | Mandipropamid | CONTROL | CONTROL |
| 20 | 0.5 | 0 | 0 | 0 | 20* |
| 10 | 0.25 | 0 | 0 | 0 | 20* |

TABLE 68

| | | AVERAGE | | | |
|---|---|---|---|---|---|
| PPM AI | | DEAD IN % | | EXPECTED | OBSERVED |
| A1 | Mefenoxam | A1 | Mefenoxam | CONTROL | CONTROL |
| 20 | 0.5 | 0 | 0 | 0 | 20* |
| 10 | 0.25 | 0 | 0 | 0 | 20* |

TABLE 69

| PPM AI | | AVERAGE | | | |
|---|---|---|---|---|---|
| | COM- | DEAD IN % | | | |
| | POUND | | COM- | EXPECTED | OBSERVED |
| A1 | III | A1 | POUND III | CONTROL | CONTROL |
| 20 | 0.5 | 0 | 90 | 90 | 100* |
| 10 | 0.25 | 0 | 50 | 50 | 50 |
| 5 | 0.125 | 0 | 20 | 20 | 20 |
| 10 | 0.5 | 0 | 90 | 90 | 70 |
| 5 | 0.25 | 0 | 50 | 50 | 50 |
| 2.5 | 0.125 | 0 | 20 | 20 | 20 |
| 5 | 0.5 | 0 | 90 | 90 | 90 |
| 2.5 | 0.25 | 0 | 50 | 50 | 50 |
| 1.25 | 0.125 | 0 | 20 | 20 | 20 |
| 2.5 | 0.5 | 0 | 90 | 90 | 90 |
| 1.25 | 0.25 | 0 | 50 | 50 | 70* |
| 0.625 | 0.125 | 0 | 20 | 20 | 50* |
| 0.3125 | 0.0625 | 0 | 0 | 0 | 50* |
| 0.15625 | 0.03125 | 0 | 0 | 0 | 20* |
| 0.078125 | 0.015625 | 0 | 0 | 0 | 20* |
| 1.25 | 0.5 | 0 | 90 | 90 | 100* |
| 0.625 | 0.25 | 0 | 50 | 50 | 100* |
| 0.3125 | 0.125 | 0 | 20 | 20 | 90* |
| 0.15625 | 0.0625 | 0 | 0 | 0 | 50* |
| 0.078125 | 0.03125 | 0 | 0 | 0 | 50* |
| 0.0390625 | 0.015625 | 0 | 0 | 0 | 50* |

TABLE 70

| | | AVERAGE | | | |
|---|---|---|---|---|---|
| PPM AI | | DEAD IN % | | | |
| | COMPOUND | | COM- | EXPECTED | OBSERVED |
| A1 | IV | A1 | POUND IV | CONTROL | CONTROL |
| 20 | 0.5 | 0 | 0 | 0 | 20* |
| 20 | 2 | 0 | 50 | 50 | 20 |
| 10 | 2 | 0 | 50 | 50 | 20 |
| 5 | 2 | 0 | 50 | 50 | 50 |

TABLE 71

| | | AVERAGE | | | |
|---|---|---|---|---|---|
| PPM AI | | DEAD IN % | | | |
| | Azoxy- | | Azoxy- | EXPECTED | OBSERVED |
| A5 | strobin | A5 | strobin | CONTROL | CONTROL |
| 0.3125 | 0.0078125 | 0 | 70 | 70 | 90* |
| 0.15625 | 0.00390625 | 0 | 0 | 0 | 0 |
| 0.15625 | 0.0078125 | 0 | 70 | 70 | 70 |
| 0.078125 | 0.0078125 | 0 | 70 | 70 | 70 |
| 0.0390625 | 0.0078125 | 0 | 70 | 70 | 70 |

TABLE 72

| | | AVERAGE | | | |
|---|---|---|---|---|---|
| PPM AI | | DEAD IN % | | EXPECTED | OBSERVED |
| A5 | Difenoconazole | A5 | Difenoconazole | CONTROL | CONTROL |
| 5 | 1 | 0 | 0 | 0 | 20* |
| 2.5 | 1 | 0 | 0 | 0 | 20* |

TABLE 73

| | | AVERAGE | | | |
|---|---|---|---|---|---|
| PPM AI | | DEAD IN % | | | |
| | COMPOUND | | COM- | EXPECTED | OBSERVED |
| A5 | III | A5 | POUND III | CONTROL | CONTROL |
| 20 | 0.5 | 0 | 90 | 90 | 100* |
| 10 | 0.25 | 0 | 50 | 50 | 20 |
| 10 | 0.5 | 0 | 90 | 90 | 100* |
| 5 | 0.25 | 0 | 50 | 50 | 50 |
| 2.5 | 0.125 | 0 | 20 | 20 | 20 |
| 5 | 0.5 | 0 | 90 | 90 | 100* |
| 2.5 | 0.25 | 0 | 50 | 50 | 50 |
| 1.25 | 0.125 | 0 | 20 | 20 | 0 |
| 2.5 | 0.5 | 0 | 90 | 90 | 100* |
| 1.25 | 0.25 | 0 | 50 | 50 | 70* |
| 0.625 | 0.125 | 0 | 20 | 20 | 0 |
| 1.25 | 0.5 | 0 | 90 | 90 | 100* |
| 0.625 | 0.25 | 0 | 50 | 50 | 90* |
| 0.3125 | 0.125 | 0 | 20 | 20 | 0 |

TABLE 74

| | | AVERAGE | | | |
|---|---|---|---|---|---|
| PPM AI | | DEAD IN % | | | |
| | COMPOUND | | COMPOUND | EXPECTED | OBSERVED |
| A5 | IV | A5 | IV | CONTROL | CONTROL |
| 20 | 2 | 0 | 50 | 50 | 20 |
| 10 | 2 | 0 | 50 | 50 | 20 |
| 5 | 2 | 0 | 50 | 50 | 20 |

TABLE 75

| | | AVERAGE | | | |
|---|---|---|---|---|---|
| | | DEAD IN % | | | |
| PPM AI | | | Azoxy- | EXPECTED | OBSERVED |
| A6 | Azoxystrobin | A6 | strobin | CONTROL | CONTROL |
| 0.625 | 0.015625 | 0 | 90 | 90 | 90 |
| 0.3125 | 0.0078125 | 0 | 20 | 20 | 20 |
| 0.3125 | 0.015625 | 0 | 90 | 90 | 90 |
| 0.15625 | 0.0078125 | 0 | 20 | 20 | 20 |
| 0.078125 | 0.00390625 | 0 | 0 | 0 | 0 |
| 0.15625 | 0.015625 | 0 | 90 | 90 | 90 |
| 0.078125 | 0.0078125 | 0 | 20 | 20 | 20 |
| 0.078125 | 0.015625 | 0 | 90 | 90 | 90 |
| 0.0390625 | 0.0078125 | 0 | 20 | 20 | 20 |
| 0.0390625 | 0.015625 | 0 | 90 | 90 | 90 |

TABLE 76

| | | AVERAGE | | | |
|---|---|---|---|---|---|
| PPM AI | | DEAD IN % | | EXPECTED | OBSERVED |
| A6 | Isopyrazam | A6 | Isopyrazam | CONTROL | CONTROL |
| 20 | 0.5 | 0 | 20 | 20 | 20 |
| 10 | 0.25 | 0 | 0 | 0 | 20* |
| 20 | 1 | 0 | 50 | 50 | 50 |
| 10 | 0.5 | 0 | 20 | 20 | 20 |
| 10 | 1 | 0 | 50 | 50 | 50 |
| 5 | 0.5 | 0 | 20 | 20 | 20 |
| 5 | 1 | 0 | 50 | 50 | 50 |
| 2.5 | 0.5 | 0 | 20 | 20 | 20 |
| 2.5 | 1 | 0 | 50 | 50 | 50 |
| 1.25 | 0.5 | 0 | 20 | 20 | 20 |

TABLE 77

| PPM AI | | AVERAGE | | | |
|---|---|---|---|---|---|
| | Chlo- | DEAD IN % | | EXPECTED | OBSERVED |
| A6 | rothalonil | A6 | Chlorothalonil | CONTROL | CONTROL |
| 10 | 0.25 | 0 | 20 | 20 | 20 |
| 5 | 0.25 | 0 | 20 | 20 | 20 |
| 2.5 | 0.25 | 0 | 20 | 20 | 20 |
| 1.25 | 0.25 | 0 | 20 | 20 | 20 |
| 0.625 | 0.25 | 0 | 20 | 20 | 20 |

TABLE 78

| PPM AI | | AVERAGE | | | |
|---|---|---|---|---|---|
| | Di- | DEAD IN % | | EXPECTED | OBSERVED |
| A6 | fenoconazole | A6 | Difenoconazole | CONTROL | CONTROL |
| 20 | 0.5 | 0 | 0 | 0 | 20* |
| 20 | 1 | 0 | 50 | 50 | 50 |
| 10 | 1 | 0 | 50 | 50 | 50 |
| 5 | 1 | 0 | 50 | 50 | 50 |
| 2.5 | 1 | 0 | 50 | 50 | 50 |

TABLE 79

| PPM AI | | AVERAGE | | | |
|---|---|---|---|---|---|
| | COM- | DEAD IN % | | | |
| | POUND | | COMPOUND | EXPECTED | OBSERVED |
| A6 | III | A6 | III | CONTROL | CONTROL |
| 20 | 0.5 | 0 | 90 | 90 | 100* |
| 10 | 0.25 | 0 | 70 | 70 | 70 |
| 5 | 0.125 | 0 | 50 | 50 | 20 |
| 2.5 | 0.0625 | 0 | 20 | 20 | 0 |
| 10 | 0.5 | 0 | 90 | 90 | 100* |
| 5 | 0.25 | 0 | 70 | 70 | 70 |
| 2.5 | 0.125 | 0 | 50 | 50 | 20 |
| 1.25 | 0.0625 | 0 | 20 | 20 | 0 |
| 5 | 0.5 | 0 | 90 | 90 | 100* |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 2.5 | 0.5 | 0 | 90 | 90 | 100 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 1.25 | 0.5 | 0 | 90 | 90 | 100* |
| 0.625 | 0.25 | 0 | 70 | 70 | 90* |
| 0.3125 | 0.125 | 0 | 50 | 50 | 50 |
| 0.15625 | 0.0625 | 0 | 20 | 20 | 20 |

TABLE 80

| | | AVERAGE | | | |
|---|---|---|---|---|---|
| PPM AI | | DEAD IN % | | | |
| | COM- | | COMPOUND | EXPECTED | OBSERVED |
| A6 | POUND IV | A6 | IV | CONTROL | CONTROL |
| 20 | 1 | 0 | 20 | 20 | 20 |
| 20 | 2 | 0 | 70 | 70 | 50 |
| 10 | 1 | 0 | 20 | 20 | 20 |
| 10 | 2 | 0 | 70 | 70 | 70 |
| 5 | 1 | 0 | 20 | 20 | 20 |
| 5 | 2 | 0 | 70 | 70 | 70 |
| 2.5 | 1 | 0 | 20 | 20 | 20 |

TABLE 81

| | | | | EX- | OB- |
|---|---|---|---|---|---|
| | | AVERAGE | | PECTED | SERVED |
| PPM AI | | DEAD IN % | | CON- | CON- |
| A8 | Azoxystrobin | A8 | Azoxystrobin | TROL | TROL |
| 0.625 | 0.015625 | 0 | 90 | 90 | 90 |
| 0.3125 | 0.0078125 | 0 | 20 | 20 | 20 |
| 0.15625 | 0.00390625 | 0 | 20 | 20 | 0 |
| 0.3125 | 0.015625 | 0 | 90 | 90 | 90 |
| 0.15625 | 0.0078125 | 0 | 20 | 20 | 20 |
| 0.078125 | 0.00390625 | 0 | 20 | 20 | 0 |
| 0.15625 | 0.015625 | 0 | 90 | 90 | 50 |
| 0.078125 | 0.0078125 | 0 | 20 | 20 | 20 |
| 0.0390625 | 0.00390625 | 0 | 20 | 20 | 0 |
| 0.078125 | 0.015625 | 0 | 90 | 90 | 90 |
| 0.0390625 | 0.0078125 | 0 | 20 | 20 | 20 |
| 0.0390625 | 0.015625 | 0 | 90 | 90 | 90 |

TABLE 82

| | | AVERAGE | | | |
|---|---|---|---|---|---|
| PPM AI | | DEAD IN % | | EXPECTED | OBSERVED |
| A8 | Isopyrazam | A8 | Isopyrazam | CONTROL | CONTROL |
| 20 | 1 | 0 | 20 | 20 | 0 |
| 20 | 2 | 0 | 20 | 20 | 0 |
| 10 | 1 | 0 | 20 | 20 | 0 |
| 10 | 2 | 0 | 20 | 20 | 0 |
| 5 | 1 | 0 | 20 | 20 | 0 |
| 5 | 2 | 0 | 20 | 20 | 0 |
| 2.5 | 1 | 0 | 20 | 20 | 0 |

TABLE 83

| | | | | | OB- |
|---|---|---|---|---|---|
| | | AVERAGE | | | SERVED |
| PPM AI | | DEAD IN % | | EXPECTED | CON- |
| A8 | Chlorothalonil | A8 | Chlorothalonil | CONTROL | TROL |
| 10 | 0.25 | 0 | 50 | 50 | 20 |
| 5 | 0.25 | 0 | 50 | 50 | 20 |
| 2.5 | 0.25 | 0 | 50 | 50 | 20 |
| 1.25 | 0.25 | 0 | 50 | 50 | 20 |
| 0.625 | 0.25 | 0 | 50 | 50 | 20 |

TABLE 84

| | | AVERAGE | | | |
|---|---|---|---|---|---|
| PPM AI | | DEAD IN % | | EXPECTED | OBSERVED |
| A8 | Difenoconazole | A8 | Difenoconazole | CONTROL | CONTROL |
| 20 | 1 | 0 | 50 | 50 | 20 |
| 10 | 1 | 0 | 50 | 50 | 20 |
| 5 | 1 | 0 | 50 | 50 | 20 |
| 2.5 | 1 | 0 | 50 | 50 | 50 |

TABLE 85

| | | AVERAGE | | | |
|---|---|---|---|---|---|
| PPM AI | | DEAD IN % | | | |
| | COM- | | COM- | EXPECTED | OBSERVED |
| A8 | POUND III | A8 | POUND III | CONTROL | CONTROL |
| 10 | 0.25 | 0 | 70 | 70 | 70 |
| 5 | 0.125 | 0 | 50 | 50 | 20 |
| 2.5 | 0.0625 | 0 | 20 | 20 | 0 |
| 5 | 0.25 | 0 | 70 | 70 | 70 |
| 2.5 | 0.125 | 0 | 50 | 50 | 20 |
| 1.25 | 0.0625 | 0 | 20 | 20 | 0 |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 20 |

TABLE 86

| | | AVERAGE | | | |
|---|---|---|---|---|---|
| PPM AI | | DEAD IN % | | | |
| | COMPOUND | | COMPOUND | EXPECTED | OBSERVED |
| A8 | IV | A8 | IV | CONTROL | CONTROL |
| 20 | 1 | 0 | 20 | 20 | 20 |
| 20 | 2 | 0 | 70 | 70 | 70 |
| 10 | 1 | 0 | 20 | 20 | 20 |
| 10 | 2 | 0 | 70 | 70 | 70 |
| 5 | 1 | 0 | 20 | 20 | 20 |
| 5 | 2 | 0 | 70 | 70 | 70 |
| 2.5 | 1 | 0 | 20 | 20 | 20 |

Botrvtis cinerea (Gray Mould):

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours. Application rates are shown in the Tables.

TABLE 87

| | | AVERAGE | | | |
|---|---|---|---|---|---|
| PPM AI | | DEAD IN % | | EXPECTED | OBSERVED |
| A1 | Azoxystrobin | A1 | Azoxystrobin | CONTROL | CONTROL |
| 20 | 0.5 | 0 | 20 | 20 | 20 |
| 10 | 0.25 | 0 | 0 | 0 | 20* |
| 20 | 1 | 0 | 20 | 20 | 20 |
| 10 | 0.5 | 0 | 20 | 20 | 20 |
| 20 | 2 | 0 | 20 | 20 | 20 |
| 10 | 1 | 0 | 20 | 20 | 20 |
| 5 | 0.5 | 0 | 20 | 20 | 20 |
| 10 | 2 | 0 | 20 | 20 | 20 |
| 5 | 1 | 0 | 20 | 20 | 20 |
| 2.5 | 0.5 | 0 | 20 | 20 | 0 |
| 5 | 2 | 0 | 20 | 20 | 20 |
| 2.5 | 1 | 0 | 20 | 20 | 20 |
| 1.25 | 0.5 | 0 | 20 | 20 | 20 |

TABLE 88

| | | | | | OB- |
|---|---|---|---|---|---|
| | | AVERAGE | | | SERVED |
| PPM AI | | DEAD IN % | | EXPECTED | CON- |
| A1 | Isopyrazam | A1 | Isopyrazam | CONTROL | TROL |
| 20 | 0.5 | 0 | 50 | 50 | 50 |
| 10 | 0.25 | 0 | 50 | 50 | 50 |
| 5 | 0.125 | 0 | 50 | 50 | 50 |
| 2.5 | 0.0625 | 0 | 50 | 50 | 20 |
| 1.25 | 0.03125 | 0 | 20 | 20 | 20 |
| 0.625 | 0.015625 | 0 | 20 | 20 | 20 |
| 0.3125 | 0.0078125 | 0 | 20 | 20 | 0 |
| 20 | 1 | 0 | 70 | 70 | 70 |
| 10 | 0.5 | 0 | 50 | 50 | 50 |
| 5 | 0.25 | 0 | 50 | 50 | 50 |
| 2.5 | 0.125 | 0 | 50 | 50 | 20 |
| 1.25 | 0.0625 | 0 | 50 | 50 | 20 |
| 0.625 | 0.03125 | 0 | 20 | 20 | 20 |
| 0.3125 | 0.015625 | 0 | 20 | 20 | 20 |
| 20 | 2 | 0 | 90 | 90 | 90 |
| 10 | 1 | 0 | 70 | 70 | 70 |
| 5 | 0.5 | 0 | 50 | 50 | 50 |
| 2.5 | 0.25 | 0 | 50 | 50 | 50 |
| 1.25 | 0.125 | 0 | 50 | 50 | 50 |
| 0.625 | 0.0625 | 0 | 50 | 50 | 20 |
| 0.3125 | 0.03125 | 0 | 20 | 20 | 20 |
| 0.15625 | 0.015625 | 0 | 20 | 20 | 20 |
| 0.078125 | 0.0078125 | 0 | 20 | 20 | 20 |
| 0.0390625 | 0.00390625 | 0 | 0 | 0 | 20* |
| 10 | 2 | 0 | 90 | 90 | 90 |
| 5 | 1 | 0 | 70 | 70 | 70 |
| 2.5 | 0.5 | 0 | 50 | 50 | 50 |
| 1.25 | 0.25 | 0 | 50 | 50 | 50 |
| 0.625 | 0.125 | 0 | 50 | 50 | 20 |
| 0.3125 | 0.0625 | 0 | 50 | 50 | 20 |
| 0.15625 | 0.03125 | 0 | 20 | 20 | 20 |
| 0.078125 | 0.015625 | 0 | 20 | 20 | 20 |
| 0.0390625 | 0.0078125 | 0 | 20 | 20 | 20 |
| 5 | 2 | 0 | 90 | 90 | 90 |
| 2.5 | 1 | 0 | 70 | 70 | 70 |
| 1.25 | 0.5 | 0 | 50 | 50 | 50 |
| 0.625 | 0.25 | 0 | 50 | 50 | 50 |
| 0.3125 | 0.125 | 0 | 50 | 50 | 50 |
| 0.15625 | 0.0625 | 0 | 50 | 50 | 20 |
| 0.078125 | 0.03125 | 0 | 20 | 20 | 20 |
| 0.0390625 | 0.015625 | 0 | 20 | 20 | 20 |

TABLE 89

| | | AVERAGE | | | |
|---|---|---|---|---|---|
| PPM AI | | DEAD IN % | | EXPECTED | OBSERVED |
| A1 | Chlorothalonil | A1 | Chlorothalonil | CONTROL | CONTROL |
| 20 | 0.5 | 0 | 0 | 0 | 0 |
| 10 | 0.5 | 0 | 0 | 0 | 20* |
| 5 | 0.5 | 0 | 0 | 0 | 20* |
| 2.5 | 0.5 | 0 | 0 | 0 | 20* |
| 1.25 | 0.5 | 0 | 0 | 0 | 20* |

TABLE 90

| | | AVERAGE | | | |
|---|---|---|---|---|---|
| PPM AI | | DEAD IN % | | EXPECTED | OBSERVED |
| A1 | Cyproconazole | A1 | Cyproconazole | CONTROL | CONTROL |
| 20 | 0.5 | 0 | 20 | 20 | 20 |
| 20 | 1 | 0 | 90 | 90 | 90 |
| 10 | 0.5 | 0 | 20 | 20 | 20 |
| 10 | 1 | 0 | 90 | 90 | 100* |
| 5 | 0.5 | 0 | 20 | 20 | 20 |
| 5 | 1 | 0 | 90 | 90 | 100* |
| 2.5 | 0.5 | 0 | 20 | 20 | 20 |
| 2.5 | 1 | 0 | 90 | 90 | 100* |
| 1.25 | 0.5 | 0 | 20 | 20 | 20 |

TABLE 91

| | | AVERAGE | | | |
|---|---|---|---|---|---|
| PPM AI | | DEAD IN % | | EXPECTED | OBSERVED |
| A1 | Difenoconazole | A1 | Difenoconazole | CONTROL | CONTROL |
| 20 | 1 | 0 | 90 | 90 | 50 |
| 20 | 2 | 0 | 100 | 100 | 90 |
| 10 | 1 | 0 | 90 | 90 | 50 |
| 5 | 1 | 0 | 90 | 90 | 50 |
| 2.5 | 1 | 0 | 90 | 90 | 70 |
| 1.25 | 0.5 | 0 | 0 | 0 | 20* |

TABLE 92

| | | AVERAGE | | | OB- |
|---|---|---|---|---|---|
| PPM AI | | DEAD IN % | | | SERVED |
| | COM- | | COM- | EXPECTED | CON- |
| A1 | POUND III | A1 | POUND III | CONTROL | TROL |
| 20 | 0.5 | 0 | 50 | 50 | 50 |
| 10 | 0.25 | 0 | 50 | 50 | 50 |
| 5 | 0.125 | 0 | 20 | 20 | 20 |
| 2.5 | 0.0625 | 0 | 20 | 20 | 20 |
| 1.25 | 0.03125 | 0 | 20 | 20 | 20 |
| 20 | 1 | 0 | 70 | 70 | 70 |
| 10 | 0.5 | 0 | 50 | 50 | 50 |
| 5 | 0.25 | 0 | 50 | 50 | 50 |
| 2.5 | 0.125 | 0 | 20 | 20 | 20 |
| 1.25 | 0.0625 | 0 | 20 | 20 | 20 |
| 0.625 | 0.03125 | 0 | 20 | 20 | 20 |
| 20 | 2 | 0 | 90 | 90 | 90 |
| 10 | 1 | 0 | 70 | 70 | 70 |
| 5 | 0.5 | 0 | 50 | 50 | 50 |
| 2.5 | 0.25 | 0 | 50 | 50 | 50 |
| 1.25 | 0.125 | 0 | 20 | 20 | 20 |
| 0.625 | 0.0625 | 0 | 20 | 20 | 20 |
| 0.3125 | 0.03125 | 0 | 20 | 20 | 20 |
| 10 | 2 | 0 | 90 | 90 | 90 |
| 5 | 1 | 0 | 70 | 70 | 70 |
| 2.5 | 0.5 | 0 | 50 | 50 | 50 |
| 1.25 | 0.25 | 0 | 50 | 50 | 50 |
| 0.625 | 0.125 | 0 | 20 | 20 | 50* |
| 0.3125 | 0.0625 | 0 | 20 | 20 | 20 |
| 0.15625 | 0.03125 | 0 | 20 | 20 | 20 |
| 0.078125 | 0.015625 | 0 | 0 | 0 | 20* |
| 0.0390625 | 0.0078125 | 0 | 0 | 0 | 20* |
| 5 | 2 | 0 | 90 | 90 | 90 |
| 2.5 | 1 | 0 | 70 | 70 | 70 |
| 1.25 | 0.5 | 0 | 50 | 50 | 70* |
| 0.625 | 0.25 | 0 | 50 | 50 | 70* |
| 0.3125 | 0.125 | 0 | 20 | 20 | 70* |
| 0.15625 | 0.0625 | 0 | 20 | 20 | 50* |
| 0.078125 | 0.03125 | 0 | 20 | 20 | 50* |
| 0.0390625 | 0.015625 | 0 | 0 | 0 | 50* |

TABLE 93

| | | AVERAGE | | | |
|---|---|---|---|---|---|
| PPM AI | | DEAD IN % | | | |
| | COM- | | COM- | EXPECTED | OBSERVED |
| A1 | POUND IV | A1 | POUND IV | CONTROL | CONTROL |
| 20 | 0.5 | 0 | 50 | 50 | 50 |
| 10 | 0.25 | 0 | 50 | 50 | 50 |
| 5 | 0.125 | 0 | 20 | 20 | 20 |
| 2.5 | 0.0625 | 0 | 20 | 20 | 20 |
| 20 | 1 | 0 | 50 | 50 | 50 |
| 10 | 0.5 | 0 | 50 | 50 | 50 |
| 5 | 0.25 | 0 | 50 | 50 | 50 |
| 2.5 | 0.125 | 0 | 20 | 20 | 20 |
| 1.25 | 0.0625 | 0 | 20 | 20 | 20 |
| 20 | 2 | 0 | 70 | 70 | 70 |
| 10 | 1 | 0 | 50 | 50 | 50 |
| 5 | 0.5 | 0 | 50 | 50 | 50 |
| 2.5 | 0.25 | 0 | 50 | 50 | 50 |
| 1.25 | 0.125 | 0 | 20 | 20 | 20 |
| 0.625 | 0.0625 | 0 | 20 | 20 | 20 |
| 10 | 2 | 0 | 70 | 70 | 70 |
| 5 | 1 | 0 | 50 | 50 | 50 |
| 2.5 | 0.5 | 0 | 50 | 50 | 50 |
| 1.25 | 0.25 | 0 | 50 | 50 | 50 |
| 0.625 | 0.125 | 0 | 20 | 20 | 20 |
| 0.3125 | 0.0625 | 0 | 20 | 20 | 0 |
| 5 | 2 | 0 | 70 | 70 | ne |
| 2.5 | 1 | 0 | 50 | 50 | ne |
| 1.25 | 0.5 | 0 | 50 | 50 | ne |
| 0.625 | 0.25 | 0 | 50 | 50 | ne |
| 0.3125 | 0.125 | 0 | 20 | 20 | 50* |
| 0.15625 | 0.0625 | 0 | 20 | 20 | 20 |
| 0.078125 | 0.03125 | 0 | 0 | 0 | 20* |

TABLE 94

| | | AVERAGE | | | |
|---|---|---|---|---|---|
| PPM AI | | DEAD IN % | | EXPECTED | OBSERVED |
| A5 | Azoxystrobin | A5 | Azoxystrobin | CONTROL | CONTROL |
| 20 | 0.5 | 0 | 50 | 50 | 50 |
| 20 | 1 | 0 | 50 | 50 | 50 |
| 10 | 0.5 | 0 | 50 | 50 | 50 |
| 20 | 2 | 0 | 50 | 50 | 50 |
| 10 | 1 | 0 | 50 | 50 | 50 |
| 5 | 0.5 | 0 | 50 | 50 | 50 |
| 10 | 2 | 0 | 50 | 50 | 70* |
| 5 | 1 | 0 | 50 | 50 | 50 |
| 2.5 | 0.5 | 0 | 50 | 50 | 50 |
| 5 | 2 | 0 | 50 | 50 | 70* |
| 2.5 | 1 | 0 | 50 | 50 | 50 |
| 1.25 | 0.5 | 0 | 50 | 50 | 50 |

TABLE 95

| PPM AI | | AVERAGE | | | |
|---|---|---|---|---|---|
| | Iso- | DEAD IN % | | EXPECTED | OBSERVED |
| A5 | pyrazam | A5 | Isopyrazam | CONTROL | CONTROL |
| 20 | 0.5 | 0 | 70 | 70 | 70 |
| 10 | 0.25 | 0 | 50 | 50 | 50 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 0.625 | 0.015625 | 0 | 0 | 0 | 20* |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 0.3125 | 0.015625 | 0 | 0 | 0 | 20* |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 0 | 0 | 20* |

TABLE 96

| PPM AI | | AVERAGE | | | |
|---|---|---|---|---|---|
| | Chlo- | DEAD IN % | | EXPECTED | OBSERVED |
| A5 | rothalonil | A5 | Chlorothalonil | CONTROL | CONTROL |
| 10 | 0.25 | 0 | 50 | 50 | 50 |
| 5 | 0.25 | 0 | 50 | 50 | 50 |
| 2.5 | 0.25 | 0 | 50 | 50 | 50 |
| 1.25 | 0.25 | 0 | 50 | 50 | 70* |
| 0.625 | 0.25 | 0 | 50 | 50 | 70* |
| 0.3125 | 0.125 | 0 | 0 | 0 | 20* |

TABLE 97

| PPM AI | | AVERAGE | | | |
|---|---|---|---|---|---|
| | Cypro- | DEAD IN % | | EXPECTED | OBSERVED |
| A5 | conazole | A5 | Cyproconazole | CONTROL | CONTROL |
| 20 | 0.5 | 0 | 20 | 20 | 20 |
| 20 | 1 | 0 | 100 | 100 | 90 |
| 10 | 0.5 | 0 | 20 | 20 | 20 |
| 10 | 1 | 0 | 100 | 100 | 90 |
| 5 | 0.5 | 0 | 20 | 20 | 20 |
| 5 | 1 | 0 | 100 | 100 | 90 |
| 2.5 | 0.5 | 0 | 20 | 20 | 20 |
| 2.5 | 1 | 0 | 100 | 100 | 90 |
| 1.25 | 0.5 | 0 | 20 | 20 | 20 |

TABLE 98

| PPM AI | | AVERAGE | | | |
|---|---|---|---|---|---|
| | Di- | DEAD N % | | EXPECTED | OBSERVED |
| A5 | fenoconazole | A5 | Difenoconazole | CONTROL | CONTROL |
| 20 | 0.5 | 0 | 20 | 20 | 0 |
| 20 | 1 | 0 | 90 | 90 | 90 |
| 10 | 0.5 | 0 | 20 | 20 | 20 |
| 10 | 1 | 0 | 90 | 90 | 90 |
| 5 | 0.5 | 0 | 20 | 20 | 50* |
| 5 | 1 | 0 | 90 | 90 | 100* |
| 2.5 | 0.5 | 0 | 20 | 20 | 50* |
| 2.5 | 1 | 0 | 90 | 90 | 100* |
| 1.25 | 0.5 | 0 | 20 | 20 | 50* |

TABLE 99

| PPM AI | | AVERAGE | | | |
|---|---|---|---|---|---|
| | COM- | DEAD IN % | | | |
| | POUND | | COM- | EXPECTED | OBSERVED |
| A5 | III | A5 | POUND III | CONTROL | CONTROL |
| 20 | 0.5 | 0 | 90 | 90 | 90 |
| 10 | 0.25 | 0 | 70 | 70 | 70 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 0.625 | 0.015625 | 0 | 20 | 20 | 20 |
| 10 | 0.5 | 0 | 90 | 90 | 90 |
| 5 | 0.25 | 0 | 70 | 70 | 70 |
| 2.5 | 0.125 | 0 | 50 | 50 | 50 |
| 1.25 | 0.0625 | 0 | 20 | 20 | 50* |
| 0.625 | 0.03125 | 0 | 20 | 20 | 20 |
| 0.3125 | 0.015625 | 0 | 20 | 20 | 20 |
| 5 | 0.5 | 0 | 90 | 90 | 90 |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 0.15625 | 0.015625 | 0 | 20 | 20 | 0 |
| 2.5 | 0.5 | 0 | 90 | 90 | 90 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 50* |
| 0.15625 | 0.03125 | 0 | 20 | 20 | 20 |
| 0.078125 | 0.015625 | 0 | 20 | 20 | 20 |
| 1.25 | 0.5 | 0 | 90 | 90 | 90 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 50* |
| 0.078125 | 0.03125 | 0 | 20 | 20 | 20 |
| 0.0390625 | 0.015625 | 0 | 20 | 20 | 20 |

TABLE 100

| PPM AI | | AVERAGE | | | |
|---|---|---|---|---|---|
| | COM- | DEAD IN % | | | |
| | POUND | | COMPOUND | EXPECTED | OBSERVED |
| A5 | IV | A5 | IV | CONTROL | CONTROL |
| 20 | 0.5 | 0 | 70 | 70 | 70 |
| 10 | 0.25 | 0 | 50 | 50 | 50 |
| 5 | 0.125 | 0 | 20 | 20 | 20 |
| 2.5 | 0.0625 | 0 | 20 | 20 | 20 |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 20 | 20 | 20 |
| 1.25 | 0.0625 | 0 | 20 | 20 | 20 |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 20 | 20 | 20 |
| 0.625 | 0.0625 | 0 | 20 | 20 | 20 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 20 | 20 | 20 |
| 0.3125 | 0.0625 | 0 | 20 | 20 | 20 |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 20 | 20 | 20 |
| 0.15625 | 0.0625 | 0 | 20 | 20 | 20 |

TABLE 101

| PPM AI | | AVERAGE | | | |
|---|---|---|---|---|---|
| | Azoxy- | DEAD IN % | | EXPECTED | OBSERVED |
| A6 | strobin | A6 | Azoxystrobin | CONTROL | CONTROL |
| 20 | 0.5 | 0 | 50 | 50 | 50 |
| 10 | 0.25 | 0 | 20 | 20 | 20 |
| 20 | 1 | 0 | 50 | 50 | 50 |
| 10 | 0.5 | 0 | 50 | 50 | 50 |
| 5 | 0.25 | 0 | 20 | 20 | 20 |
| 20 | 2 | 0 | 50 | 50 | 50 |
| 10 | 1 | 0 | 50 | 50 | 50 |
| 5 | 0.5 | 0 | 50 | 50 | 50 |
| 2.5 | 0.25 | 0 | 20 | 20 | 20 |
| 10 | 2 | 0 | 50 | 50 | 50 |
| 5 | 1 | 0 | 50 | 50 | 50 |
| 2.5 | 0.5 | 0 | 50 | 50 | 50 |
| 1.25 | 0.25 | 0 | 20 | 20 | 20 |
| 5 | 2 | 0 | 50 | 50 | 50 |
| 2.5 | 1 | 0 | 50 | 50 | 50 |
| 1.25 | 0.5 | 0 | 50 | 50 | 50 |
| 0.625 | 0.25 | 0 | 20 | 20 | 20 |

TABLE 102

| PPM AI | | AVERAGE | | | |
|---|---|---|---|---|---|
| | Iso- | DEAD IN % | | EXPECTED | OBSERVED |
| A6 | pyrazam | A6 | Isopyrazam | CONTROL | CONTROL |
| 20 | 0.5 | 0 | 70 | 70 | 70 |
| 10 | 0.25 | 0 | 50 | 50 | 50 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 0.625 | 0.015625 | 0 | 20 | 20 | 20 |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 0.3125 | 0.015625 | 0 | 20 | 20 | 20 |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 0.15625 | 0.015625 | 0 | 20 | 20 | 20 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 0.078125 | 0.015625 | 0 | 20 | 20 | 20 |
| 0.0390625 | 0.0078125 | 0 | 0 | 0 | 20* |
| 5 | 2 | 0 | 100 | 100 | 90 |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 20 | 20 | 20 |

TABLE 103

| PPM AI | | AVERAGE | | | |
|---|---|---|---|---|---|
| | Chloro- | DEAD IN % | | EXPECTED | OBSERVED |
| A6 | thalonil | A6 | Chlorothalonil | CONTROL | CONTROL |
| 10 | 0.25 | 0 | 50 | 50 | 50 |
| 5 | 0.25 | 0 | 50 | 50 | 50 |
| 2.5 | 0.25 | 0 | 50 | 50 | 50 |
| 1.25 | 0.25 | 0 | 50 | 50 | 50 |
| 0.625 | 0.25 | 0 | 50 | 50 | 50 |
| 0.3125 | 0.125 | 0 | 0 | 0 | 20* |

TABLE 104

| PPM AI | | AVERAGE | | | |
|---|---|---|---|---|---|
| | Cypro- | DEAD IN % | | EXPECTED | OBSERVED |
| A6 | conazole | A6 | Cyproconazole | CONTROL | CONTROL |
| 20 | 0.5 | 0 | 50 | 50 | 20 |
| 10 | 0.5 | 0 | 50 | 50 | 20 |
| 5 | 0.5 | 0 | 50 | 50 | 20 |
| 2.5 | 0.5 | 0 | 50 | 50 | 20 |
| 1.25 | 0.5 | 0 | 50 | 50 | 50 |

TABLE 105

| | | AVERAGE | | | |
|---|---|---|---|---|---|
| PPM AI | | DEAD IN % | | EXPECTED | OBSERVED |
| A6 | Difenoconazole | A6 | Difenoconazole | CONTROL | CONTROL |
| 20 | 0.5 | 0 | 50 | 50 | 50 |
| 10 | 0.25 | 0 | 0 | 0 | 20* |
| 20 | 1 | 0 | 90 | 90 | 100* |
| 10 | 0.5 | 0 | 50 | 50 | 50 |
| 10 | 1 | 0 | 90 | 90 | 100* |
| 5 | 0.5 | 0 | 50 | 50 | 50 |
| 5 | 1 | 0 | 90 | 90 | 100* |
| 2.5 | 0.5 | 0 | 50 | 50 | 50 |
| 2.5 | 1 | 0 | 90 | 90 | 100* |
| 1.25 | 0.5 | 0 | 50 | 50 | 50 |

TABLE 106

| PPM AI | | AVERAGE | | | |
|---|---|---|---|---|---|
| | COM- | DEAD IN % | | | |
| | POUND | | COM- | EXPECTED | OBSERVED |
| A6 | III | A6 | POUND III | CONTROL | CONTROL |
| 20 | 0.5 | 0 | 70 | 70 | 70 |
| 10 | 0.25 | 0 | 50 | 50 | 50 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 10 | 1 | 0 | 90 | 90 | 90 |
| 5 | 0.5 | 0 | 70 | 70 | 70 |
| 2.5 | 0.25 | 0 | 50 | 50 | 70* |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 0 | 0 | 20* |

TABLE 107

| PPM AI | | AVERAGE | | | |
|---|---|---|---|---|---|
| | COM- | DEAD IN % | | | |
| | POUND | | COMPOUND | EXPECTED | OBSERVED |
| A6 | IV | A6 | IV | CONTROL | CONTROL |
| 20 | 0.5 | 0 | 70 | 70 | 70 |
| 10 | 0.25 | 0 | 50 | 50 | 50 |
| 5 | 0.125 | 0 | 20 | 20 | 20 |
| 2.5 | 0.0625 | 0 | 20 | 20 | 20 |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 20 | 20 | 20 |
| 1.25 | 0.0625 | 0 | 20 | 20 | 20 |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 20 | 20 | 20 |
| 0.625 | 0.0625 | 0 | 20 | 20 | 0 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 20 | 20 | 20 |
| 0.3125 | 0.0625 | 0 | 20 | 20 | 20 |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 20 | 20 | 20 |
| 0.15625 | 0.0625 | 0 | 20 | 20 | 20 |

TABLE 108

| PPM AI | | AVERAGE | | | |
|---|---|---|---|---|---|
| | Azoxy- | DEAD IN % | | EXPECTED | OBSERVED |
| A8 | strobin | A8 | Azoxystrobin | CONTROL | CONTROL |
| 20 | 0.5 | 0 | 50 | 50 | 50 |
| 10 | 0.25 | 0 | 20 | 20 | 20 |
| 20 | 1 | 0 | 50 | 50 | 50 |
| 10 | 0.5 | 0 | 50 | 50 | 50 |
| 5 | 0.25 | 0 | 20 | 20 | 20 |
| 20 | 2 | 0 | 50 | 50 | 50 |
| 10 | 1 | 0 | 50 | 50 | 50 |
| 5 | 0.5 | 0 | 50 | 50 | 50 |
| 2.5 | 0.25 | 0 | 20 | 20 | 20 |
| 10 | 2 | 0 | 50 | 50 | 50 |
| 5 | 1 | 0 | 50 | 50 | 50 |
| 2.5 | 0.5 | 0 | 50 | 50 | 50 |
| 1.25 | 0.25 | 0 | 20 | 20 | 20 |
| 5 | 2 | 0 | 50 | 50 | 50 |
| 2.5 | 1 | 0 | 50 | 50 | 50 |
| 1.25 | 0.5 | 0 | 50 | 50 | 50 |
| 0.625 | 0.25 | 0 | 20 | 20 | 20 |

TABLE 109

| | | AVERAGE | | | |
|---|---|---|---|---|---|
| PPM AI | | DEAD IN % | | | |
| | Iso- | | Iso- | EXPECTED | OBSERVED |
| A8 | pyrazam | A8 | pyrazam | CONTROL | CONTROL |
| 20 | 0.5 | 0 | 70 | 70 | 70 |
| 10 | 0.25 | 0 | 50 | 50 | 50 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 0.625 | 0.015625 | 0 | 20 | 20 | 20 |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 0.3125 | 0.015625 | 0 | 20 | 20 | 20 |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 0.15625 | 0.015625 | 0 | 20 | 20 | 20 |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 0.078125 | 0.015625 | 0 | 20 | 20 | 20 |
| 0.0390625 | 0.0078125 | 0 | 0 | 0 | 20* |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 20 | 20 | 20 |

TABLE 110

| | | AVERAGE | | | |
|---|---|---|---|---|---|
| PPM AI | | DEAD IN % | | | |
| | Chloro- | | Chloro- | EXPECTED | OBSERVED |
| A8 | thalonil | A8 | thalonil | CONTROL | CONTROL |
| 5 | 0.125 | 0 | 20 | 20 | 50* |
| 2.5 | 0.125 | 0 | 20 | 20 | 50* |
| 1.25 | 0.125 | 0 | 20 | 20 | 50* |
| 0.625 | 0.125 | 0 | 20 | 20 | 20 |
| 0.3125 | 0.125 | 0 | 20 | 20 | 20 |

TABLE 111

| | | AVERAGE | | | |
|---|---|---|---|---|---|
| PPM AI | | DEAD IN % | | EXPECTED | OBSERVED |
| A8 | Cyproconazole | A8 | Cyproconazole | CONTROL | CONTROL |
| 20 | 0.5 | 0 | 20 | 20 | 20 |
| 10 | 0.5 | 0 | 20 | 20 | 20 |
| 5 | 0.5 | 0 | 20 | 20 | 20 |
| 2.5 | 0.5 | 0 | 20 | 20 | 20 |
| 1.25 | 0.5 | 0 | 20 | 20 | 20 |

TABLE 112

| | | AVERAGE | | | |
|---|---|---|---|---|---|
| PPM AI | | DEAD IN % | | EXPECTED | OBSERVED |
| A8 | Difenoconazole | A8 | Difenoconazole | CONTROL | CONTROL |
| 20 | 0.5 | 0 | 50 | 50 | 50 |
| 10 | 0.25 | 0 | 0 | 0 | 20* |
| 20 | 1 | 0 | 90 | 90 | 90 |
| 10 | 0.5 | 0 | 50 | 50 | 50 |
| 10 | 1 | 0 | 90 | 90 | 90 |
| 5 | 0.5 | 0 | 50 | 50 | 50 |
| 5 | 1 | 0 | 90 | 90 | 100* |
| 2.5 | 0.5 | 0 | 50 | 50 | 50 |
| 2.5 | 1 | 0 | 90 | 90 | 100* |
| 1.25 | 0.5 | 0 | 50 | 50 | 50 |

TABLE 113

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A8 | III | A8 | III | CONTROL | CONTROL |
| 20 | 0.5 | 0 | 70 | 70 | 70 |
| 10 | 0.25 | 0 | 50 | 50 | 50 |
| 5 | 0.125 | 0 | 50 | 50 | 50 |
| 2.5 | 0.0625 | 0 | 20 | 20 | 20 |
| 1.25 | 0.03125 | 0 | 20 | 20 | 20 |
| 0.625 | 0.015625 | 0 | 20 | 20 | 20 |
| 10 | 0.5 | 0 | 70 | 70 | 70 |
| 5 | 0.25 | 0 | 50 | 50 | 50 |
| 2.5 | 0.125 | 0 | 50 | 50 | 50 |
| 1.25 | 0.0625 | 0 | 20 | 20 | 20 |
| 0.625 | 0.03125 | 0 | 20 | 20 | 20 |
| 0.3125 | 0.015625 | 0 | 20 | 20 | 20 |
| 5 | 0.5 | 0 | 70 | 70 | 70 |
| 2.5 | 0.25 | 0 | 50 | 50 | 50 |
| 1.25 | 0.125 | 0 | 50 | 50 | 50 |
| 0.625 | 0.0625 | 0 | 20 | 20 | 20 |
| 0.3125 | 0.03125 | 0 | 20 | 20 | 20 |
| 0.15625 | 0.015625 | 0 | 20 | 20 | 20 |
| 5 | 1 | 0 | 100 | 100 | 90 |
| 2.5 | 0.5 | 0 | 70 | 70 | 70 |
| 1.25 | 0.25 | 0 | 50 | 50 | 50 |
| 0.625 | 0.125 | 0 | 50 | 50 | 50 |
| 0.3125 | 0.0625 | 0 | 20 | 20 | 20 |
| 0.15625 | 0.03125 | 0 | 20 | 20 | 20 |
| 0.078125 | 0.015625 | 0 | 20 | 20 | 20 |
| 2.5 | 1 | 0 | 100 | 100 | 90 |
| 1.25 | 0.5 | 0 | 70 | 70 | 70 |
| 0.625 | 0.25 | 0 | 50 | 50 | 50 |
| 0.3125 | 0.125 | 0 | 50 | 50 | 50 |
| 0.15625 | 0.0625 | 0 | 20 | 20 | 20 |
| 0.078125 | 0.03125 | 0 | 20 | 20 | 20 |
| 0.0390625 | 0.015625 | 0 | 20 | 20 | 20 |

TABLE 114

| | | AVERAGE | | | |
|---|---|---|---|---|---|
| PPM AI | | DEAD IN % | | | |
| | COM- | | COM- | EXPECTED | OBSERVED |
| A8 | POUND IV | A8 | POUND IV | CONTROL | CONTROL |
| 20 | 0.5 | 0 | 70 | 70 | 70 |
| 10 | 0.25 | 0 | 50 | 50 | 50 |
| 5 | 0.125 | 0 | 20 | 20 | 20 |
| 2.5 | 0.0625 | 0 | 20 | 20 | 20 |
| 1.25 | 0.03125 | 0 | 0 | 0 | 20* |
| 20 | 1 | 0 | 90 | 90 | 90 |
| 10 | 0.5 | 0 | 70 | 70 | 70 |
| 5 | 0.25 | 0 | 50 | 50 | 50 |
| 2.5 | 0.125 | 0 | 20 | 20 | 20 |
| 1.25 | 0.0625 | 0 | 20 | 20 | 20 |
| 0.625 | 0.03125 | 0 | 0 | 0 | 20* |
| 10 | 1 | 0 | 90 | 90 | 90 |
| 5 | 0.5 | 0 | 70 | 70 | 70 |
| 2.5 | 0.25 | 0 | 50 | 50 | 50 |
| 1.25 | 0.125 | 0 | 20 | 20 | 20 |
| 0.625 | 0.0625 | 0 | 20 | 20 | 20 |
| 0.3125 | 0.03125 | 0 | 0 | 0 | 20* |
| 5 | 1 | 0 | 90 | 90 | 90 |
| 2.5 | 0.5 | 0 | 70 | 70 | 70 |
| 1.25 | 0.25 | 0 | 50 | 50 | 50 |
| 0.625 | 0.125 | 0 | 20 | 20 | 20 |
| 0.3125 | 0.0625 | 0 | 20 | 20 | 20 |
| 0.15625 | 0.03125 | 0 | 0 | 0 | 20* |
| 2.5 | 1 | 0 | 90 | 90 | 90 |
| 1.25 | 0.5 | 0 | 70 | 70 | 70 |
| 0.625 | 0.25 | 0 | 50 | 50 | 50 |
| 0.3125 | 0.125 | 0 | 20 | 20 | 20 |
| 0.15625 | 0.0625 | 0 | 20 | 20 | 20 |
| 0.078125 | 0.03125 | 0 | 0 | 0 | 20* |

Glomerella lagenarium (syn. Colletotrichum lagenarium), Anthracnose of Cucurbits:

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours at 620 nm. Application rates are shown in the Tables.

TABLE 115

| | | AVERAGE | | | |
|---|---|---|---|---|---|
| PPM AI | | DEAD IN % | | | |
| | Azoxy- | | Azoxy- | EXPECTED | OBSERVED |
| A1 | strobin | A1 | strobin | CONTROL | CONTROL |
| 1.25 | 0.03125 | 0 | 50 | 50 | 70* |
| 0.625 | 0.015625 | 0 | 20 | 20 | 20 |
| 0.625 | 0.03125 | 0 | 50 | 50 | 70* |
| 0.3125 | 0.015625 | 0 | 20 | 20 | 20 |
| 0.3125 | 0.03125 | 0 | 50 | 50 | 70* |
| 0.15625 | 0.015625 | 0 | 20 | 20 | 20 |
| 0.15625 | 0.03125 | 0 | 50 | 50 | 70* |
| 0.078125 | 0.015625 | 0 | 20 | 20 | 50* |
| 0.0390625 | 0.0078125 | 0 | 0 | 0 | 20* |
| 0.078125 | 0.03125 | 0 | 50 | 50 | 100* |
| 0.0390625 | 0.015625 | 0 | 20 | 20 | 90* |

TABLE 116

| | | AVERAGE | | | |
|---|---|---|---|---|---|
| PPM AI | | DEAD IN % | | EXPECTED | OBSERVED |
| A1 | Isopyrazam | A1 | Isopyrazam | CONTROL | CONTROL |
| 20 | 2 | 0 | 50 | 50 | 0 |
| 10 | 2 | 0 | 50 | 50 | 0 |
| 5 | 2 | 0 | 50 | 50 | 20 |

TABLE 117

| | | AVERAGE | | | |
|---|---|---|---|---|---|
| PPM AI | | DEAD IN % | | | |
| | Chloro- | | Chloro- | EXPECTED | OBSERVED |
| A1 | thalonil | A1 | thalonil | CONTROL | CONTROL |
| 20 | 0.5 | 0 | 70 | 70 | 100* |
| 10 | 0.25 | 0 | 50 | 50 | 50 |
| 5 | 0.125 | 0 | 20 | 20 | 20 |
| 10 | 0.5 | 0 | 70 | 70 | 100* |
| 5 | 0.25 | 0 | 50 | 50 | 50 |
| 2.5 | 0.125 | 0 | 20 | 20 | 20 |
| 5 | 0.5 | 0 | 70 | 70 | 90* |
| 2.5 | 0.25 | 0 | 50 | 50 | 50 |
| 1.25 | 0.125 | 0 | 20 | 20 | 20 |
| 2.5 | 0.5 | 0 | 70 | 70 | 100* |
| 1.25 | 0.25 | 0 | 50 | 50 | 50 |
| 0.625 | 0.125 | 0 | 20 | 20 | 20 |
| 1.25 | 0.5 | 0 | 70 | 70 | 100* |
| 0.625 | 0.25 | 0 | 50 | 50 | 50 |
| 0.3125 | 0.125 | 0 | 20 | 20 | 20 |

TABLE 118

| | | AVERAGE | | | |
|---|---|---|---|---|---|
| PPM AI | | DEAD IN % | | EXPECTED | OBSERVED |
| A1 | Difenoconazole | A1 | Difenoconazole | CONTROL | CONTROL |
| 20 | 0.5 | 0 | 20 | 20 | 20 |
| 20 | 1 | 0 | 20 | 20 | 20 |
| 10 | 0.5 | 0 | 20 | 20 | 20 |
| 20 | 2 | 0 | 50 | 50 | 50 |
| 10 | 1 | 0 | 20 | 20 | 20 |
| 5 | 0.5 | 0 | 20 | 20 | 20 |
| 10 | 2 | 0 | 50 | 50 | 50 |
| 5 | 1 | 0 | 20 | 20 | 20 |
| 2.5 | 0.5 | 0 | 20 | 20 | 0 |
| 5 | 2 | 0 | 50 | 50 | 50 |
| 2.5 | 1 | 0 | 20 | 20 | 20 |
| 1.25 | 0.5 | 0 | 20 | 20 | 20 |

TABLE 119

| | | AVERAGE | | | |
|---|---|---|---|---|---|
| PPM AI | | DEAD IN % | | | |
| | COM- | | COM- | EXPECTED | OBSERVED |
| A1 | POUND III | A1 | POUND III | CONTROL | CONTROL |
| 5 | 0.125 | 0 | 50 | 50 | 100* |
| 2.5 | 0.0625 | 0 | 20 | 20 | 50* |
| 1.25 | 0.03125 | 0 | 0 | 0 | 20* |
| 2.5 | 0.125 | 0 | 50 | 50 | 90* |
| 1.25 | 0.0625 | 0 | 20 | 20 | 50* |
| 0.625 | 0.03125 | 0 | 0 | 0 | 20* |
| 1.25 | 0.125 | 0 | 50 | 50 | 90* |
| 0.625 | 0.0625 | 0 | 20 | 20 | 50* |
| 0.3125 | 0.03125 | 0 | 0 | 0 | 20* |
| 0.625 | 0.125 | 0 | 50 | 50 | 100* |
| 0.3125 | 0.0625 | 0 | 20 | 20 | 100* |
| 0.15625 | 0.03125 | 0 | 0 | 0 | 100* |
| 0.078125 | 0.015625 | 0 | 0 | 0 | 100* |
| 0.0390625 | 0.0078125 | 0 | 0 | 0 | 90* |
| 0.3125 | 0.125 | 0 | 50 | 50 | 100* |
| 0.15625 | 0.0625 | 0 | 20 | 20 | 100* |
| 0.078125 | 0.03125 | 0 | 0 | 0 | 100* |
| 0.0390625 | 0.015625 | 0 | 0 | 0 | 100* |

TABLE 120

| PPM AI | | AVERAGE | | | |
|---|---|---|---|---|---|
| A5 | Azoystrobin | DEAD IN % | | EXPECTED | OBSERVED |
| 2.5 | 0.0625 | 0 | 70 | 70 | 90* |
| 1.25 | 0.03125 | 0 | 50 | 50 | 50 |
| 0.625 | 0.015625 | 0 | 50 | 50 | 20 |
| 1.25 | 0.0625 | 0 | 70 | 70 | 90* |
| 0.625 | 0.03125 | 0 | 50 | 50 | 50 |
| 0.3125 | 0.015625 | 0 | 50 | 50 | 20 |
| 0.625 | 0.0625 | 0 | 70 | 70 | 100* |
| 0.3125 | 0.03125 | 0 | 50 | 50 | 90* |
| 0.15625 | 0.015625 | 0 | 50 | 50 | 0 |
| 0.3125 | 0.0625 | 0 | 70 | 70 | 90* |
| 0.15625 | 0.03125 | 0 | 50 | 50 | 70* |
| 0.078125 | 0.015625 | 0 | 50 | 50 | 50 |
| 0.0390625 | 0.0078125 | 0 | 0 | 0 | 20* |
| 0.15625 | 0.0625 | 0 | 70 | 70 | 90* |
| 0.078125 | 0.03125 | 0 | 50 | 50 | 70* |
| 0.0390625 | 0.015625 | 0 | 50 | 50 | 50 |

TABLE 121

| | | AVERAGE | | | |
|---|---|---|---|---|---|
| PPM AI | | DEAD IN % | | EXPECTED | OBSERVED |
| A5 | Isopyrazam | A5 | Isopyrazam | CONTROL | CONTROL |
| 10 | 2 | 0 | 20 | 20 | 50* |
| 5 | 2 | 0 | 20 | 20 | 20 |

TABLE 122

| | | AVERAGE | | | |
|---|---|---|---|---|---|
| PPM AI | | DEAD IN % | | | |
| | Chloro- | | Chloro- | EXPECTED | OBSERVED |
| A5 | thalonil | A5 | thalonil | CONTROL | CONTROL |
| 10 | 0.25 | 0 | 90 | 90 | 90 |
| 5 | 0.125 | 0 | 50 | 50 | 50 |
| 5 | 0.25 | 0 | 90 | 90 | 100* |
| 2.5 | 0.125 | 0 | 50 | 50 | 50 |
| 2.5 | 0.25 | 0 | 90 | 90 | 100* |
| 1.25 | 0.125 | 0 | 50 | 50 | 70* |
| 1.25 | 0.25 | 0 | 90 | 90 | 100* |
| 0.625 | 0.125 | 0 | 50 | 50 | 50 |
| 0.625 | 0.25 | 0 | 90 | 90 | 100* |
| 0.3125 | 0.125 | 0 | 50 | 50 | 50 |

TABLE 123

| | | AVERAGE | | | |
|---|---|---|---|---|---|
| PPM AI | | DEAD IN % | | | |
| | Difeno- | | Difeno- | EXPECTED | OBSERVED |
| A5 | conazole | A5 | conazole | CONTROL | CONTROL |
| 20 | 1 | 0 | 20 | 20 | 20 |
| 20 | 2 | 0 | 50 | 50 | 50 |
| 10 | 1 | 0 | 20 | 20 | 20 |
| 10 | 2 | 0 | 50 | 50 | 50 |
| 5 | 1 | 0 | 20 | 20 | 20 |
| 5 | 2 | 0 | 50 | 50 | 50 |
| 2.5 | 1 | 0 | 20 | 20 | 20 |

TABLE 124

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A5 | III | A5 | III | CONTROL | CONTROL |
| 2.5 | 0.0625 | 0 | 100 | 100 | 70 |
| 1.25 | 0.03125 | 0 | 20 | 20 | 20 |
| 1.25 | 0.0625 | 0 | 100 | 100 | 90 |
| 0.625 | 0.03125 | 0 | 20 | 20 | 20 |
| 0.625 | 0.0625 | 0 | 100 | 100 | 90 |
| 0.3125 | 0.03125 | 0 | 20 | 20 | 20 |
| 0.3125 | 0.0625 | 0 | 100 | 100 | 90 |
| 0.15625 | 0.03125 | 0 | 20 | 20 | 0 |
| 0.15625 | 0.0625 | 0 | 100 | 100 | 90 |
| 0.078125 | 0.03125 | 0 | 20 | 20 | 20 |

TABLE 125

| | | AVERAGE | | | |
|---|---|---|---|---|---|
| PPM AI | | DEAD IN % | | EXPECTED | OBSERVED |
| A6 | Isopyrazam | A6 | Isopyrazam | CONTROL | CONTROL |
| 20 | 2 | 0 | 20 | 20 | 20 |
| 10 | 2 | 0 | 20 | 20 | 20 |
| 5 | 2 | 0 | 20 | 20 | 20 |

TABLE 126

| | | AVERAGE | | | |
|---|---|---|---|---|---|
| PPM AI | | DEAD IN % | | EXPECTED | OBSERVED |
| A6 | Difenoconazole | A6 | Difenoconazole | CONTROL | CONTROL |
| 20 | 1 | 0 | 20 | 20 | 20 |
| 20 | 2 | 0 | 20 | 20 | 20 |
| 10 | 1 | 0 | 20 | 20 | 20 |
| 10 | 2 | 0 | 20 | 20 | 0 |
| 5 | 1 | 0 | 20 | 20 | 0 |
| 5 | 2 | 0 | 20 | 20 | 50* |
| 2.5 | 1 | 0 | 20 | 20 | 0 |

TABLE 127

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A6 | III | A6 | III | CONTROL | CONTROL |
| 0.15625 | 0.03125 | 0 | 0 | 0 | 100* |
| 0.078125 | 0.015625 | 0 | 0 | 0 | 20* |
| 0.15625 | 0.0625 | 0 | 100 | 100 | 90 |
| 0.078125 | 0.03125 | 0 | 0 | 0 | 20* |

TABLE 128

| | | AVERAGE | | | |
|---|---|---|---|---|---|
| PPM AI | | DEAD IN % | | EXPECTED | OBSERVED |
| A8 | Isopyrazam | A8 | Isopyrazam | CONTROL | CONTROL |
| 20 | 2 | 0 | 20 | 20 | 20 |
| 10 | 2 | 0 | 20 | 20 | 20 |
| 5 | 2 | 0 | 20 | 20 | 20 |

TABLE 129

| | | AVERAGE | | | |
|---|---|---|---|---|---|
| PPM AI | | DEAD IN % | | | |
| | Chloro- | | Chloro- | EXPECTED | OBSERVED |
| A8 | thalonil | A8 | thalonil | CONTROL | CONTROL |
| 10 | 0.25 | 0 | 90 | 90 | 20 |
| 5 | 0.25 | 0 | 90 | 90 | 50 |
| 2.5 | 0.25 | 0 | 90 | 90 | 100* |
| 1.25 | 0.25 | 0 | 90 | 90 | 100* |
| 0.625 | 0.25 | 0 | 90 | 90 | 70 |

TABLE 130

| | | AVERAGE | | | |
|---|---|---|---|---|---|
| PPM AI | | DEAD IN % | | EXPECTED | OBSERVED |
| A8 | Difenoconazole | A8 | Difenoconazole | CONTROL | CONTROL |
| 20 | 1 | 0 | 20 | 20 | 0 |
| 20 | 2 | 0 | 20 | 20 | 20 |
| 10 | 1 | 0 | 20 | 20 | 0 |
| 10 | 2 | 0 | 20 | 20 | 20 |
| 5 | 1 | 0 | 20 | 20 | 0 |
| 5 | 2 | 0 | 20 | 20 | 20 |
| 2.5 | 1 | 0 | 20 | 20 | 0 |

TABLE 131

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A8 | III | A8 | III | CONTROL | CONTROL |
| 2.5 | 0.0625 | 0 | 70 | 70 | 100* |
| 1.25 | 0.0625 | 0 | 70 | 70 | 70 |
| 0.625 | 0.0625 | 0 | 70 | 70 | 90* |
| 0.3125 | 0.0625 | 0 | 70 | 70 | 50 |
| 0.15625 | 0.03125 | 0 | 0 | 0 | 20* |
| 0.15625 | 0.0625 | 0 | 70 | 70 | 50 |
| 0.078125 | 0.03125 | 0 | 0 | 0 | 20* |

Septoria tritici (Leaf Blotch):

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours. Application rates are shown in the Tables.

TABLE 132

| | | AVERAGE | | | |
|---|---|---|---|---|---|
| PPM AI | | DEAD IN % | | | |
| | Azoxy- | | Azoxy- | EXPECTED | OBSERVED |
| A1 | strobin | A1 | strobin | CONTROL | CONTROL |
| 5 | 0.125 | 0 | 90 | 90 | 100* |
| 2.5 | 0.0625 | 0 | 70 | 70 | 90* |
| 1.25 | 0.03125 | 0 | 50 | 50 | 50 |
| 0.625 | 0.015625 | 0 | 20 | 20 | 20 |
| 2.5 | 0.125 | 0 | 90 | 90 | 100* |
| 1.25 | 0.0625 | 0 | 70 | 70 | 70 |
| 0.625 | 0.03125 | 0 | 50 | 50 | 50 |
| 0.3125 | 0.015625 | 0 | 20 | 20 | 20 |
| 1.25 | 0.125 | 0 | 90 | 90 | 90 |
| 0.625 | 0.0625 | 0 | 70 | 70 | 70 |
| 0.3125 | 0.03125 | 0 | 50 | 50 | 50 |
| 0.15625 | 0.015625 | 0 | 20 | 20 | 20 |
| 0.625 | 0.125 | 0 | 90 | 90 | 90 |
| 0.3125 | 0.0625 | 0 | 70 | 70 | 70 |
| 0.15625 | 0.03125 | 0 | 50 | 50 | 50 |
| 0.078125 | 0.015625 | 0 | 20 | 20 | 50* |
| 0.0390625 | 0.0078125 | 0 | 0 | 0 | 20* |
| 0.3125 | 0.125 | 0 | 90 | 90 | 100* |
| 0.15625 | 0.0625 | 0 | 70 | 70 | 90* |
| 0.078125 | 0.03125 | 0 | 50 | 50 | 90* |
| 0.0390625 | 0.015625 | 0 | 20 | 20 | 70* |

TABLE 133

| | | AVERAGE | | | |
|---|---|---|---|---|---|
| PPM AI | | DEAD IN % | | | |
| | Iso- | | Iso- | EXPECTED | OBSERVED |
| A1 | pyrazam | A1 | pyrazam | CONTROL | CONTROL |
| 20 | 0.5 | 0 | 90 | 90 | 100* |
| 10 | 0.25 | 0 | 70 | 70 | 90* |
| 5 | 0.125 | 0 | 50 | 50 | 90* |
| 2.5 | 0.0625 | 0 | 20 | 20 | 50* |
| 1.25 | 0.03125 | 0 | 0 | 0 | 20* |
| 10 | 0.5 | 0 | 90 | 90 | 90 |
| 5 | 0.25 | 0 | 70 | 70 | 70 |
| 2.5 | 0.125 | 0 | 50 | 50 | 50 |
| 1.25 | 0.0625 | 0 | 20 | 20 | 20 |
| 5 | 0.5 | 0 | 90 | 90 | 90 |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 2.5 | 0.5 | 0 | 90 | 90 | 90 |
| 1.25 | 0.25 | 0 | 70 | 70 | 70 |
| 0.625 | 0.125 | 0 | 50 | 50 | 70* |
| 0.3125 | 0.0625 | 0 | 20 | 20 | 50* |
| 0.15625 | 0.03125 | 0 | 0 | 0 | 20* |
| 0.078125 | 0.015625 | 0 | 0 | 0 | 20* |
| 0.0390625 | 0.0078125 | 0 | 0 | 0 | 20* |
| 1.25 | 0.5 | 0 | 90 | 90 | 90 |
| 0.625 | 0.25 | 0 | 70 | 70 | 90* |
| 0.3125 | 0.125 | 0 | 50 | 50 | 70* |
| 0.15625 | 0.0625 | 0 | 20 | 20 | 50* |
| 0.078125 | 0.03125 | 0 | 0 | 0 | 50* |
| 0.0390625 | 0.015625 | 0 | 0 | 0 | 20* |

TABLE 134

| | | AVERAGE | | | |
|---|---|---|---|---|---|
| PPM AI | | DEAD IN % | | EXPECTED | OBSERVED |
| A1 | Chlorothalonil | A1 | Chlorothalonil | CONTROL | CONTROL |
| 20 | 1 | 0 | 70 | 70 | 100* |
| 10 | 1 | 0 | 70 | 70 | 100* |
| 5 | 1 | 0 | 70 | 70 | 100* |
| 2.5 | 1 | 0 | 70 | 70 | 100* |

TABLE 135

| | | AVERAGE | | | |
|---|---|---|---|---|---|
| PPM AI | | DEAD IN % | | | |
| | Cypro- | | Cypro- | EXPECTED | OBSERVED |
| A1 | conazole | A1 | conazole | CONTROL | CONTROL |
| 20 | 0.5 | 0 | 50 | 50 | 70* |
| 10 | 0.25 | 0 | 0 | 0 | 50* |
| 20 | 1 | 0 | 70 | 70 | 70 |
| 10 | 0.5 | 0 | 50 | 50 | 50 |
| 5 | 0.25 | 0 | 0 | 0 | 20* |
| 20 | 2 | 0 | 70 | 70 | 70 |
| 10 | 1 | 0 | 70 | 70 | 70 |
| 5 | 0.5 | 0 | 50 | 50 | 50 |
| 10 | 2 | 0 | 70 | 70 | 70 |
| 5 | 1 | 0 | 70 | 70 | 70 |
| 2.5 | 0.5 | 0 | 50 | 50 | 50 |
| 5 | 2 | 0 | 70 | 70 | 70 |
| 2.5 | 1 | 0 | 70 | 70 | 70 |
| 1.25 | 0.5 | 0 | 50 | 50 | 50 |
| 0.625 | 0.25 | 0 | 0 | 0 | 20* |

TABLE 136

| | | AVERAGE | | | |
|---|---|---|---|---|---|
| PPM AI | | DEAD IN % | | | |
| | Difeno- | | Difeno- | EXPECTED | OBSERVED |
| A1 | conazole | A1 | conazole | CONTROL | CONTROL |
| 20 | 0.5 | 0 | 90 | 90 | 90 |
| 10 | 0.25 | 0 | 70 | 70 | 90* |
| 5 | 0.125 | 0 | 70 | 70 | 90* |
| 2.5 | 0.0625 | 0 | 50 | 50 | 70* |
| 1.25 | 0.03125 | 0 | 20 | 20 | 20 |
| 20 | 1 | 0 | 90 | 90 | 90 |
| 10 | 0.5 | 0 | 90 | 90 | 90 |
| 5 | 0.25 | 0 | 70 | 70 | 70 |
| 2.5 | 0.125 | 0 | 70 | 70 | 70 |
| 1.25 | 0.0625 | 0 | 50 | 50 | 50 |
| 0.625 | 0.03125 | 0 | 20 | 20 | 20 |
| 20 | 2 | 0 | 90 | 90 | 90 |
| 10 | 1 | 0 | 90 | 90 | 90 |
| 5 | 0.5 | 0 | 90 | 90 | 90 |
| 2.5 | 0.25 | 0 | 70 | 70 | 70 |
| 1.25 | 0.125 | 0 | 70 | 70 | 70 |
| 0.625 | 0.0625 | 0 | 50 | 50 | 50 |
| 0.3125 | 0.03125 | 0 | 20 | 20 | 20 |
| 10 | 2 | 0 | 90 | 90 | 90 |
| 5 | 1 | 0 | 90 | 90 | 90 |
| 2.5 | 0.5 | 0 | 90 | 90 | 90 |
| 1.25 | 0.25 | 0 | 70 | 70 | 90* |
| 0.625 | 0.125 | 0 | 70 | 70 | 90* |
| 0.3125 | 0.0625 | 0 | 50 | 50 | 90* |
| 0.15625 | 0.03125 | 0 | 20 | 20 | 70* |
| 0.078125 | 0.015625 | 0 | 0 | 0 | 70* |
| 0.0390625 | 0.0078125 | 0 | 0 | 0 | 50* |
| 5 | 2 | 0 | 90 | 90 | 90 |
| 2.5 | 1 | 0 | 90 | 90 | 90 |
| 1.25 | 0.5 | 0 | 90 | 90 | 90 |
| 0.625 | 0.25 | 0 | 70 | 70 | 90* |
| 0.3125 | 0.125 | 0 | 70 | 70 | 90* |
| 0.15625 | 0.0625 | 0 | 50 | 50 | 70* |
| 0.078125 | 0.03125 | 0 | 20 | 20 | 70* |
| 0.0390625 | 0.015625 | 0 | 0 | 0 | 70* |

TABLE 137

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A1 | III | A1 | III | CONTROL | CONTROL |
| 20 | 0.5 | 0 | 70 | 70 | 90* |
| 10 | 0.25 | 0 | 50 | 50 | 90* |
| 5 | 0.125 | 0 | 20 | 20 | 70* |
| 2.5 | 0.0625 | 0 | 0 | 0 | 50* |
| 1.25 | 0.03125 | 0 | 0 | 0 | 20* |
| 20 | 1 | 0 | 90 | 90 | 100* |
| 10 | 0.5 | 0 | 70 | 70 | 90* |
| 5 | 0.25 | 0 | 50 | 50 | 70* |
| 2.5 | 0.125 | 0 | 20 | 20 | 50* |
| 1.25 | 0.0625 | 0 | 0 | 0 | 20* |
| 10 | 1 | 0 | 90 | 90 | 100* |
| 5 | 0.5 | 0 | 70 | 70 | 90* |
| 2.5 | 0.25 | 0 | 50 | 50 | 70* |
| 1.25 | 0.125 | 0 | 20 | 20 | 50* |
| 0.625 | 0.0625 | 0 | 0 | 0 | 20* |
| 5 | 1 | 0 | 90 | 90 | 100* |
| 2.5 | 0.5 | 0 | 70 | 70 | 90* |
| 1.25 | 0.25 | 0 | 50 | 50 | 70* |
| 0.625 | 0.125 | 0 | 20 | 20 | 70* |
| 0.3125 | 0.0625 | 0 | 0 | 0 | 50* |
| 0.15625 | 0.03125 | 0 | 0 | 0 | 20* |
| 0.078125 | 0.015625 | 0 | 0 | 0 | 20* |
| 0.0390625 | 0.0078125 | 0 | 0 | 0 | 20* |
| 2.5 | 1 | 0 | 90 | 90 | 90 |
| 1.25 | 0.5 | 0 | 70 | 70 | 90* |
| 0.625 | 0.25 | 0 | 50 | 50 | 90* |
| 0.3125 | 0.125 | 0 | 20 | 20 | 70* |
| 0.15625 | 0.0625 | 0 | 0 | 0 | 70* |
| 0.078125 | 0.03125 | 0 | 0 | 0 | 70* |
| 0.0390625 | 0.015625 | 0 | 0 | 0 | 50* |

TABLE 138

| | | AVERAGE | | | |
|---|---|---|---|---|---|
| PPM AI | | DEAD IN % | | | |
| | COM- | | COM- | | |
| | POUND | | POUND | EXPECTED | OBSERVED |
| A1 | IV | A1 | IV | CONTROL | CONTROL |
| 20 | 0.5 | 0 | 90 | 90 | 100* |
| 10 | 0.25 | 0 | 70 | 70 | 90* |
| 5 | 0.125 | 0 | 50 | 50 | 70* |
| 2.5 | 0.0625 | 0 | 20 | 20 | 20 |
| 10 | 0.5 | 0 | 90 | 90 | 90 |
| 5 | 0.25 | 0 | 70 | 70 | 90* |
| 2.5 | 0.125 | 0 | 50 | 50 | 50 |
| 1.25 | 0.0625 | 0 | 20 | 20 | 20 |
| 5 | 0.5 | 0 | 90 | 90 | 90 |
| 2.5 | 0.25 | 0 | 70 | 70 | 90* |
| 1.25 | 0.125 | 0 | 50 | 50 | 50 |
| 0.625 | 0.0625 | 0 | 20 | 20 | 20 |
| 2.5 | 0.5 | 0 | 90 | 90 | 90 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 1.25 | 0.5 | 0 | 90 | 90 | 90 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 20 |

TABLE 139

| | | | | EX- | OB- |
|---|---|---|---|---|---|
| | | AVERAGE | | PECTED | SERVED |
| PPM AI | | DEAD IN % | | CON- | CON- |
| A5 | Azoxystrobin | A5 | Azoxystrobin | TROL | TROL |
| 2.5 | 0.0625 | 0 | 70 | 70 | 70 |
| 1.25 | 0.03125 | 0 | 20 | 20 | 20 |
| 1.25 | 0.0625 | 0 | 70 | 70 | 70 |
| 0.625 | 0.03125 | 0 | 20 | 20 | 20 |
| 0.625 | 0.0625 | 0 | 70 | 70 | 70 |
| 0.3125 | 0.03125 | 0 | 20 | 20 | 20 |
| 0.3125 | 0.0625 | 0 | 70 | 70 | 90* |
| 0.15625 | 0.03125 | 0 | 20 | 20 | 20 |
| 0.15625 | 0.0625 | 0 | 70 | 70 | 70 |
| 0.078125 | 0.03125 | 0 | 20 | 20 | 20 |

TABLE 140

| | | AVERAGE | | | |
|---|---|---|---|---|---|
| PPM AI | | DEAD IN % | | EXPECTED | OBSERVED |
| A5 | Isopyrazam | A5 | Isopyrazam | CONTROL | CONTROL |
| 10 | 0.25 | 0 | 90 | 90 | 90 |
| 5 | 0.125 | 0 | 20 | 20 | 50* |
| 2.5 | 0.0625 | 0 | 20 | 20 | 20 |
| 5 | 0.25 | 0 | 90 | 90 | 90 |
| 2.5 | 0.125 | 0 | 20 | 20 | 20 |
| 1.25 | 0.0625 | 0 | 20 | 20 | 20 |
| 2.5 | 0.25 | 0 | 90 | 90 | 90 |
| 1.25 | 0.125 | 0 | 20 | 20 | 20 |
| 0.625 | 0.0625 | 0 | 20 | 20 | 20 |
| 1.25 | 0.25 | 0 | 90 | 90 | 90 |
| 0.625 | 0.125 | 0 | 20 | 20 | 50* |
| 0.3125 | 0.0625 | 0 | 20 | 20 | 20 |
| 0.625 | 0.25 | 0 | 90 | 90 | 70 |
| 0.3125 | 0.125 | 0 | 20 | 20 | 20 |

TABLE 141

| | | | | EX- | OB- |
|---|---|---|---|---|---|
| | | AVERAGE | | PECTED | SERVED |
| PPM AI | | DEAD IN % | | CON- | CON- |
| A5 | Chlorothalonil | A5 | Chlorothalonil | TROL | TROL |
| 2.5 | 0.0625 | 0 | 70 | 70 | 70 |
| 1.25 | 0.0625 | 0 | 70 | 70 | 70 |
| 0.625 | 0.0625 | 0 | 70 | 70 | 100* |
| 0.3125 | 0.0625 | 0 | 70 | 70 | 100* |
| 0.15625 | 0.0625 | 0 | 70 | 70 | 70 |
| 0.078125 | 0.03125 | 0 | 0 | 0 | 20* |

TABLE 142

| | | | | EX- | OB- |
|---|---|---|---|---|---|
| | | AVERAGE | | PECTED | SERVED |
| PPM AI | | DEAD IN % | | CON- | CON- |
| A5 | Cyproconazole | A5 | Cyproconazole | TROL | TROL |
| 10 | 0.25 | 0 | 50 | 50 | 20 |
| 5 | 0.25 | 0 | 50 | 50 | 20 |
| 2.5 | 0.25 | 0 | 50 | 50 | 20 |
| 1.25 | 0.25 | 0 | 50 | 50 | 50 |
| 0.625 | 0.25 | 0 | 50 | 50 | 20 |

TABLE 143

| | | | | EX- | OB- |
|---|---|---|---|---|---|
| | | AVERAGE | | PECTED | SERVED |
| PPM AI | | DEAD IN % | | CON- | CON- |
| A5 | Difenoconazole | A5 | Difenoconazole | TROL | TROL |
| 1.25 | 0.03125 | 0 | 70 | 70 | 70 |
| 0.625 | 0.03125 | 0 | 70 | 70 | 90* |
| 0.3125 | 0.03125 | 0 | 70 | 70 | 90* |
| 0.15625 | 0.03125 | 0 | 70 | 70 | 100* |
| 0.078125 | 0.015625 | 0 | 0 | 0 | 20* |
| 0.078125 | 0.03125 | 0 | 70 | 70 | 100* |
| 0.0390625 | 0.015625 | 0 | 0 | 0 | 20* |

TABLE 144

| | | AVERAGE | | | |
|---|---|---|---|---|---|
| PPM AI | | DEAD IN % | | | |
| | COM- | | COM- | EXPECTED | OBSERVED |
| A5 | POUND III | A5 | POUND III | CONTROL | CONTROL |
| 10 | 0.25 | 0 | 70 | 70 | 70 |
| 5 | 0.125 | 0 | 20 | 20 | 50* |
| 5 | 0.25 | 0 | 70 | 70 | 90* |
| 2.5 | 0.125 | 0 | 20 | 20 | 50* |
| 2.5 | 0.25 | 0 | 70 | 70 | 90* |
| 1.25 | 0.125 | 0 | 20 | 20 | 20 |
| 1.25 | 0.25 | 0 | 70 | 70 | 100* |
| 0.625 | 0.125 | 0 | 20 | 20 | 50* |
| 0.3125 | 0.0625 | 0 | 0 | 0 | 20* |
| 0.625 | 0.25 | 0 | 70 | 70 | 90* |
| 0.3125 | 0.125 | 0 | 20 | 20 | 50* |
| 0.15625 | 0.0625 | 0 | 0 | 0 | 20* |

TABLE 145

| PPM AI | | AVERAGE | | | |
|---|---|---|---|---|---|
| | COM- | DEAD IN % | | | |
| | POUND | | COMPOUND | EXPECTED | OBSERVED |
| A5 | IV | A5 | IV | CONTROL | CONTROL |
| 10 | 0.25 | 0 | 90 | 90 | 90 |
| 5 | 0.125 | 0 | 20 | 20 | 20 |
| 5 | 0.25 | 0 | 90 | 90 | 90 |
| 2.5 | 0.125 | 0 | 20 | 20 | 20 |
| 2.5 | 0.25 | 0 | 90 | 90 | 100* |
| 1.25 | 0.125 | 0 | 20 | 20 | 90* |
| 0.625 | 0.0625 | 0 | 0 | 0 | 20* |
| 2.5 | 0.5 | 0 | 100 | 100 | 90 |
| 1.25 | 0.25 | 0 | 90 | 90 | 70 |
| 0.625 | 0.125 | 0 | 20 | 20 | 50* |
| 0.3125 | 0.0625 | 0 | 0 | 0 | 20* |
| 0.625 | 0.25 | 0 | 90 | 90 | 70 |
| 0.3125 | 0.125 | 0 | 20 | 20 | 20 |

TABLE 146

| | | | | EX- | OB- |
|---|---|---|---|---|---|
| | | AVERAGE | | PECTED | SERVED |
| PPM AI | | DEAD IN % | | CON- | CON- |
| A6 | Azoxystrobin | A6 | Azoxystrobin | TROL | TROL |
| 5 | 0.125 | 0 | 90 | 90 | 90 |
| 2.5 | 0.0625 | 0 | 70 | 70 | 70 |
| 1.25 | 0.03125 | 0 | 50 | 50 | 50 |
| 0.625 | 0.015625 | 0 | 20 | 20 | 20 |
| 2.5 | 0.125 | 0 | 90 | 90 | 90 |
| 1.25 | 0.0625 | 0 | 70 | 70 | 70 |
| 0.625 | 0.03125 | 0 | 50 | 50 | 20 |
| 0.3125 | 0.015625 | 0 | 20 | 20 | 0 |
| 1.25 | 0.125 | 0 | 90 | 90 | 90 |
| 0.625 | 0.0625 | 0 | 70 | 70 | 70 |
| 0.3125 | 0.03125 | 0 | 50 | 50 | 20 |
| 0.15625 | 0.015625 | 0 | 20 | 20 | 0 |
| 0.625 | 0.125 | 0 | 90 | 90 | 90 |
| 0.3125 | 0.0625 | 0 | 70 | 70 | 70 |
| 0.15625 | 0.03125 | 0 | 50 | 50 | 20 |
| 0.078125 | 0.015625 | 0 | 20 | 20 | 0 |
| 0.3125 | 0.125 | 0 | 90 | 90 | 90 |
| 0.15625 | 0.0625 | 0 | 70 | 70 | 70 |
| 0.078125 | 0.03125 | 0 | 50 | 50 | 20 |
| 0.0390625 | 0.015625 | 0 | 20 | 20 | 0 |

TABLE 147

| | | AVERAGE | | | |
|---|---|---|---|---|---|
| PPM AI | | DEAD IN % | | EXPECTED | OBSERVED |
| A6 | Isopyrazam | A6 | Isopyrazam | CONTROL | CONTROL |
| 20 | 0.5 | 0 | 90 | 90 | 100* |
| 10 | 0.25 | 0 | 70 | 70 | 70 |
| 5 | 0.125 | 0 | 50 | 50 | 50 |
| 2.5 | 0.0625 | 0 | 20 | 20 | 20 |
| 10 | 0.5 | 0 | 90 | 90 | 100* |
| 5 | 0.25 | 0 | 70 | 70 | 70 |
| 2.5 | 0.125 | 0 | 50 | 50 | 50 |
| 1.25 | 0.0625 | 0 | 20 | 20 | 20 |
| 5 | 0.5 | 0 | 90 | 90 | 100* |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 2.5 | 0.5 | 0 | 90 | 90 | 90 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 1.25 | 0.5 | 0 | 90 | 90 | 90 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 20 |

TABLE 148

| | | | | EX- | OB- |
|---|---|---|---|---|---|
| | | AVERAGE | | PECTED | SERVED |
| PPM AI | | DEAD IN % | | CON- | CON- |
| A6 | Chlorothalonil | A6 | Chlorothalonil | TROL | TROL |
| 10 | 0.25 | 0 | 20 | 20 | 20 |
| 5 | 0.25 | 0 | 20 | 20 | 20 |
| 2.5 | 0.25 | 0 | 20 | 20 | 20 |
| 1.25 | 0.25 | 0 | 20 | 20 | 20 |
| 0.625 | 0.25 | 0 | 20 | 20 | 20 |

TABLE 149

| | | | | | OB- |
|---|---|---|---|---|---|
| | | AVERAGE | | | SERVED |
| PPM AI | | DEAD IN % | | EXPECTED | CON- |
| A6 | Cyproconazole | A6 | Cyproconazole | CONTROL | TROL |
| 20 | 0.5 | 0 | 50 | 50 | 70* |
| 10 | 0.25 | 0 | 20 | 20 | 20 |
| 20 | 1 | 0 | 70 | 70 | 90* |
| 10 | 0.5 | 0 | 50 | 50 | 70* |
| 5 | 0.25 | 0 | 20 | 20 | 20 |
| 20 | 2 | 0 | 90 | 90 | 90 |
| 10 | 1 | 0 | 70 | 70 | 90* |
| 5 | 0.5 | 0 | 50 | 50 | 70* |
| 2.5 | 0.25 | 0 | 20 | 20 | 20 |
| 10 | 2 | 0 | 90 | 90 | 90 |
| 5 | 1 | 0 | 70 | 70 | 90* |
| 2.5 | 0.5 | 0 | 50 | 50 | 70* |
| 1.25 | 0.25 | 0 | 20 | 20 | 20 |
| 5 | 2 | 0 | 90 | 90 | 90 |
| 2.5 | 1 | 0 | 70 | 70 | 90* |
| 1.25 | 0.5 | 0 | 50 | 50 | 70* |
| 0.625 | 0.25 | 0 | 20 | 20 | 20 |

TABLE 150

| | | | | | EX- |
|---|---|---|---|---|---|
| | | | | PECT- | OB- |
| | | AVERAGE | | ED | SERVED |
| PPM AI | | DEAD IN % | | CON- | CON- |
| A6 | Difenoconazole | A6 | Difenoconazole | TROL | TROL |
| 20 | 0.5 | 0 | 90 | 90 | 90 |
| 10 | 0.25 | 0 | 90 | 90 | 90 |
| 5 | 0.125 | 0 | 90 | 90 | 90 |
| 2.5 | 0.0625 | 0 | 70 | 70 | 90* |
| 1.25 | 0.03125 | 0 | 20 | 20 | 50* |
| 20 | 1 | 0 | 90 | 90 | 90 |
| 10 | 0.5 | 0 | 90 | 90 | 90 |
| 5 | 0.25 | 0 | 90 | 90 | 90 |
| 2.5 | 0.125 | 0 | 90 | 90 | 90 |
| 1.25 | 0.0625 | 0 | 70 | 70 | 70 |
| 0.625 | 0.03125 | 0 | 20 | 20 | 20 |
| 20 | 2 | 0 | 90 | 90 | 90 |
| 10 | 1 | 0 | 90 | 90 | 90 |
| 5 | 0.5 | 0 | 90 | 90 | 90 |
| 2.5 | 0.25 | 0 | 90 | 90 | 90 |
| 1.25 | 0.125 | 0 | 90 | 90 | 90 |
| 0.625 | 0.0625 | 0 | 70 | 70 | 70 |
| 0.3125 | 0.03125 | 0 | 20 | 20 | 20 |
| 10 | 2 | 0 | 90 | 90 | 90 |
| 5 | 1 | 0 | 90 | 90 | 90 |
| 2.5 | 0.5 | 0 | 90 | 90 | 90 |
| 1.25 | 0.25 | 0 | 90 | 90 | 90 |
| 0.625 | 0.125 | 0 | 90 | 90 | 90 |
| 0.3125 | 0.0625 | 0 | 70 | 70 | 70 |
| 0.15625 | 0.03125 | 0 | 20 | 20 | 20 |
| 5 | 2 | 0 | 90 | 90 | 90 |
| 2.5 | 1 | 0 | 90 | 90 | 90 |
| 1.25 | 0.5 | 0 | 90 | 90 | 90 |
| 0.625 | 0.25 | 0 | 90 | 90 | 90 |
| 0.3125 | 0.125 | 0 | 90 | 90 | 90 |
| 0.15625 | 0.0625 | 0 | 70 | 70 | 70 |
| 0.078125 | 0.03125 | 0 | 20 | 20 | 50* |
| 0.0390625 | 0.015625 | 0 | 0 | 0 | 20* |

TABLE 151

| | | AVERAGE | | | |
|---|---|---|---|---|---|
| PPM AI | | DEAD IN % | | | |
| | COM- | | COM- | EXPECTED | OBSERVED |
| A6 | POUND III | A6 | POUND III | CONTROL | CONTROL |
| 20 | 0.5 | 0 | 90 | 90 | 90 |
| 10 | 0.25 | 0 | 70 | 70 | 70 |
| 5 | 0.125 | 0 | 20 | 20 | 20 |
| 10 | 0.5 | 0 | 90 | 90 | 90 |
| 5 | 0.25 | 0 | 70 | 70 | 70 |
| 2.5 | 0.125 | 0 | 20 | 20 | 20 |
| 5 | 0.5 | 0 | 90 | 90 | 90 |
| 2.5 | 0.25 | 0 | 70 | 70 | 70 |
| 1.25 | 0.125 | 0 | 20 | 20 | 50* |
| 2.5 | 0.5 | 0 | 90 | 90 | 90 |
| 1.25 | 0.25 | 0 | 70 | 70 | 70 |
| 0.625 | 0.125 | 0 | 20 | 20 | 20 |
| 1.25 | 0.5 | 0 | 90 | 90 | 90 |
| 0.625 | 0.25 | 0 | 70 | 70 | 70 |
| 0.3125 | 0.125 | 0 | 20 | 20 | 20 |

TABLE 152

| PPM AI | | AVERAGE | | | |
|---|---|---|---|---|---|
| | COM- | DEAD IN % | | | |
| | POUND | | COMPOUND | EXPECTED | OBSERVED |
| A6 | IV | A6 | IV | CONTROL | CONTROL |
| 10 | 0.25 | 0 | 90 | 90 | 90 |
| 5 | 0.125 | 0 | 20 | 20 | 20 |
| 5 | 0.25 | 0 | 90 | 90 | 90 |
| 2.5 | 0.125 | 0 | 20 | 20 | 20 |
| 2.5 | 0.25 | 0 | 90 | 90 | 90 |
| 1.25 | 0.125 | 0 | 20 | 20 | 20 |
| 1.25 | 0.25 | 0 | 90 | 90 | 90 |
| 0.625 | 0.125 | 0 | 20 | 20 | 20 |
| 0.625 | 0.25 | 0 | 90 | 90 | 90 |
| 0.3125 | 0.125 | 0 | 20 | 20 | 20 |

TABLE 153

| | | | | EX- | OB- |
|---|---|---|---|---|---|
| | | AVERAGE | | PECTED | SERVED |
| PPM AI | | DEAD IN % | | CON- | CON- |
| A8 | Azoxystrobin | A8 | Azoxystrobin | TROL | TROL |
| 5 | 0.125 | 0 | 90 | 90 | 90 |
| 2.5 | 0.0625 | 0 | 70 | 70 | 70 |
| 1.25 | 0.03125 | 0 | 50 | 50 | 50 |
| 0.625 | 0.015625 | 0 | 20 | 20 | 20 |
| 2.5 | 0.125 | 0 | 90 | 90 | 90 |
| 1.25 | 0.0625 | 0 | 70 | 70 | 70 |
| 0.625 | 0.03125 | 0 | 50 | 50 | 20 |
| 1.25 | 0.125 | 0 | 90 | 90 | 90 |
| 0.625 | 0.0625 | 0 | 70 | 70 | 70 |
| 0.3125 | 0.03125 | 0 | 50 | 50 | 20 |
| 0.625 | 0.125 | 0 | 90 | 90 | 90 |
| 0.3125 | 0.0625 | 0 | 70 | 70 | 70 |
| 0.15625 | 0.03125 | 0 | 50 | 50 | 20 |
| 0.3125 | 0.125 | 0 | 90 | 90 | 90 |
| 0.15625 | 0.0625 | 0 | 70 | 70 | 70 |
| 0.078125 | 0.03125 | 0 | 50 | 50 | 20 |
| 0.0390625 | 0.015625 | 0 | 20 | 20 | 0 |

TABLE 154

| | | AVERAGE | | | |
|---|---|---|---|---|---|
| PPM AI | | DEAD IN % | | EXPECTED | OBSERVED |
| A8 | Isopyrazam | A8 | Isopyrazam | CONTROL | CONTROL |
| 20 | 0.5 | 0 | 90 | 90 | 100* |
| 10 | 0.25 | 0 | 70 | 70 | 70 |
| 5 | 0.125 | 0 | 50 | 50 | 50 |
| 2.5 | 0.0625 | 0 | 20 | 20 | 20 |
| 10 | 0.5 | 0 | 90 | 90 | 100* |
| 5 | 0.25 | 0 | 70 | 70 | 70 |
| 2.5 | 0.125 | 0 | 50 | 50 | 50 |
| 1.25 | 0.0625 | 0 | 20 | 20 | 20 |
| 5 | 0.5 | 0 | 90 | 90 | 100* |
| 2.5 | 0.25 | 0 | 70 | 70 | 70 |
| 1.25 | 0.125 | 0 | 50 | 50 | 50 |
| 0.625 | 0.0625 | 0 | 20 | 20 | 20 |
| 2.5 | 0.5 | 0 | 90 | 90 | 90 |
| 1.25 | 0.25 | 0 | 70 | 70 | 70 |
| 0.625 | 0.125 | 0 | 50 | 50 | 50 |
| 0.3125 | 0.0625 | 0 | 20 | 20 | 20 |
| 1.25 | 0.5 | 0 | 90 | 90 | 90 |
| 0.625 | 0.25 | 0 | 70 | 70 | 70 |
| 0.3125 | 0.125 | 0 | 50 | 50 | 50 |
| 0.15625 | 0.0625 | 0 | 20 | 20 | 20 |

TABLE 155

| | | AVERAGE | | | |
|---|---|---|---|---|---|
| PPM AI | | DEAD IN % | | | |
| | Chloro- | | Chloro- | EXPECTED | OBSERVED |
| A8 | thalonil | A8 | thalonil | CONTROL | CONTROL |
| 20 | 0.5 | 0 | 50 | 50 | 70* |
| 10 | 0.25 | 0 | 0 | 0 | 20* |
| 10 | 0.5 | 0 | 50 | 50 | 90* |
| 5 | 0.5 | 0 | 50 | 50 | 90* |
| 2.5 | 0.5 | 0 | 50 | 50 | 90* |
| 1.25 | 0.25 | 0 | 0 | 0 | 20* |
| 1.25 | 0.5 | 0 | 50 | 50 | 70* |
| 0.625 | 0.25 | 0 | 0 | 0 | 20* |

TABLE 156

| | | AVERAGE | | | |
|---|---|---|---|---|---|
| PPM AI | | DEAD IN % | | | |
| | Cypro- | | Cypro- | EXPECTED | OBSERVED |
| A8 | conazole | A8 | conazole | CONTROL | CONTROL |
| 20 | 0.5 | 0 | 50 | 50 | 70* |
| 10 | 0.25 | 0 | 20 | 20 | 20 |
| 20 | 1 | 0 | 70 | 70 | 90* |
| 10 | 0.5 | 0 | 50 | 50 | 50 |
| 5 | 0.25 | 0 | 20 | 20 | 20 |
| 20 | 2 | 0 | 90 | 90 | 90 |
| 10 | 1 | 0 | 70 | 70 | 90* |
| 5 | 0.5 | 0 | 50 | 50 | 50 |
| 2.5 | 0.25 | 0 | 20 | 20 | 20 |
| 10 | 2 | 0 | 90 | 90 | 90 |
| 5 | 1 | 0 | 70 | 70 | 90* |
| 2.5 | 0.5 | 0 | 50 | 50 | 50 |
| 1.25 | 0.25 | 0 | 20 | 20 | 20 |
| 5 | 2 | 0 | 90 | 90 | 90 |
| 2.5 | 1 | 0 | 70 | 70 | 70 |
| 1.25 | 0.5 | 0 | 50 | 50 | 50 |
| 0.625 | 0.25 | 0 | 20 | 20 | 20 |

TABLE 157

| | | AVERAGE | | | |
|---|---|---|---|---|---|
| PPM AI | | DEAD IN % | | | |
| | Difeno- | | Difeno- | EXPECTED | OBSERVED |
| A8 | conazole | A8 | conazole | CONTROL | CONTROL |
| 20 | 0.5 | 0 | 90 | 90 | 90 |
| 10 | 0.25 | 0 | 90 | 90 | 90 |
| 5 | 0.125 | 0 | 90 | 90 | 90 |
| 2.5 | 0.0625 | 0 | 70 | 70 | 70 |
| 1.25 | 0.03125 | 0 | 50 | 50 | 50 |
| 20 | 1 | 0 | 90 | 90 | 90 |
| 10 | 0.5 | 0 | 90 | 90 | 90 |
| 5 | 0.25 | 0 | 90 | 90 | 90 |
| 2.5 | 0.125 | 0 | 90 | 90 | 90 |
| 1.25 | 0.0625 | 0 | 70 | 70 | 70 |
| 0.625 | 0.03125 | 0 | 50 | 50 | 50 |
| 20 | 2 | 0 | 90 | 90 | 90 |
| 10 | 1 | 0 | 90 | 90 | 90 |
| 5 | 0.5 | 0 | 90 | 90 | 90 |
| 2.5 | 0.25 | 0 | 90 | 90 | 90 |
| 1.25 | 0.125 | 0 | 90 | 90 | 90 |
| 0.625 | 0.0625 | 0 | 70 | 70 | 70 |
| 0.3125 | 0.03125 | 0 | 50 | 50 | 50 |
| 10 | 2 | 0 | 90 | 90 | 90 |
| 5 | 1 | 0 | 90 | 90 | 90 |
| 2.5 | 0.5 | 0 | 90 | 90 | 90 |
| 1.25 | 0.25 | 0 | 90 | 90 | 90 |
| 0.625 | 0.125 | 0 | 90 | 90 | 90 |
| 0.3125 | 0.0625 | 0 | 70 | 70 | 70 |
| 0.15625 | 0.03125 | 0 | 50 | 50 | 20 |
| 5 | 2 | 0 | 90 | 90 | 90 |
| 2.5 | 1 | 0 | 90 | 90 | 90 |
| 1.25 | 0.5 | 0 | 90 | 90 | 90 |
| 0.625 | 0.25 | 0 | 90 | 90 | 90 |
| 0.3125 | 0.125 | 0 | 90 | 90 | 90 |
| 0.15625 | 0.0625 | 0 | 70 | 70 | 70 |
| 0.078125 | 0.03125 | 0 | 50 | 50 | 50 |

TABLE 158

| | | AVERAGE | | | |
|---|---|---|---|---|---|
| PPM AI | | DEAD IN % | | | |
| | COM- | | COM- | EXPECTED | OBSERVED |
| A8 | POUND III | A8 | POUND III | CONTROL | CONTROL |
| 20 | 0.5 | 0 | 90 | 90 | 90 |
| 10 | 0.25 | 0 | 50 | 50 | 70* |
| 5 | 0.125 | 0 | 20 | 20 | 20 |
| 10 | 0.5 | 0 | 90 | 90 | 90 |
| 5 | 0.25 | 0 | 50 | 50 | 70* |
| 2.5 | 0.125 | 0 | 20 | 20 | 20 |
| 5 | 0.5 | 0 | 90 | 90 | 90 |
| 2.5 | 0.25 | 0 | 50 | 50 | 70* |
| 1.25 | 0.125 | 0 | 20 | 20 | 50* |
| 2.5 | 0.5 | 0 | 90 | 90 | 90 |
| 1.25 | 0.25 | 0 | 50 | 50 | 70* |
| 0.625 | 0.125 | 0 | 20 | 20 | 20 |
| 1.25 | 0.5 | 0 | 90 | 90 | 70 |
| 0.625 | 0.25 | 0 | 50 | 50 | 50 |
| 0.3125 | 0.125 | 0 | 20 | 20 | 20 |

TABLE 159

| | | AVERAGE | | | |
|---|---|---|---|---|---|
| PPM AI | | DEAD IN % | | | |
| | COM- | | COM- | EXPECTED | OBSERVED |
| A8 | POUND IV | A8 | POUND IV | CONTROL | CONTROL |
| 10 | 0.25 | 0 | 90 | 90 | 90 |
| 5 | 0.125 | 0 | 20 | 20 | 20 |
| 5 | 0.25 | 0 | 90 | 90 | 90 |
| 2.5 | 0.125 | 0 | 20 | 20 | 20 |
| 2.5 | 0.25 | 0 | 90 | 90 | 90 |
| 1.25 | 0.125 | 0 | 20 | 20 | 20 |
| 1.25 | 0.25 | 0 | 90 | 90 | 90 |
| 0.625 | 0.125 | 0 | 20 | 20 | 20 |
| 0.625 | 0.25 | 0 | 90 | 90 | 70 |
| 0.3125 | 0.125 | 0 | 20 | 20 | 20 |

Mycosphaerella arachidis (syn. Cercospora arachidicola), Brown Leaf Spot of Groundnut (Peanut):

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after approximately 5-6 days at 620 nm. Application rates are shown in the Tables.

TABLE 160

| | | AVERAGE | | | |
|---|---|---|---|---|---|
| PPM AI | | DEAD IN % | | | |
| | Azoxy- | | Azoxy- | EXPECTED | OBSERVED |
| A1 | strobin | A1 | strobin | CONTROL | CONTROL |
| 5 | 0.125 | 0 | 90 | 90 | 90 |
| 2.5 | 0.0625 | 0 | 70 | 70 | 70 |
| 1.25 | 0.03125 | 0 | 50 | 50 | 50 |
| 2.5 | 0.125 | 0 | 90 | 90 | 90 |
| 1.25 | 0.0625 | 0 | 70 | 70 | 90* |
| 0.625 | 0.03125 | 0 | 50 | 50 | 50 |
| 0.625 | 0.0625 | 0 | 70 | 70 | 90* |
| 0.625 | 0.125 | 0 | 90 | 90 | 100* |
| 0.3125 | 0.0625 | 0 | 70 | 70 | 90* |
| 0.15625 | 0.03125 | 0 | 50 | 50 | 70* |
| 0.078125 | 0.015625 | 0 | 0 | 0 | 50* |
| 0.0390625 | 0.0078125 | 0 | 0 | 0 | 20* |
| 0.3125 | 0.125 | 0 | 90 | 90 | 100* |
| 0.15625 | 0.0625 | 0 | 70 | 70 | 100* |
| 0.078125 | 0.03125 | 0 | 50 | 50 | 90* |
| 0.0390625 | 0.015625 | 0 | 0 | 0 | 70* |

TABLE 161

| | | AVERAGE | | | |
|---|---|---|---|---|---|
| PPM AI | | DEAD IN % | | EXPECTED | OBSERVED |
| A1 | Isopyrazam | A1 | Isopyrazam | CONTROL | CONTROL |
| 2.5 | 0.0625 | 0 | 70 | 70 | 70 |
| 1.25 | 0.03125 | 0 | 20 | 20 | 20 |
| 1.25 | 0.0625 | 0 | 70 | 70 | 70 |
| 0.625 | 0.03125 | 0 | 20 | 20 | 20 |
| 0.625 | 0.0625 | 0 | 70 | 70 | 90* |
| 0.3125 | 0.03125 | 0 | 20 | 20 | 50* |
| 0.15625 | 0.015625 | 0 | 0 | 0 | 20* |
| 0.078125 | 0.0078125 | 0 | 0 | 0 | 20* |
| 0.3125 | 0.0625 | 0 | 70 | 70 | 100* |
| 0.15625 | 0.03125 | 0 | 20 | 20 | 90* |
| 0.078125 | 0.015625 | 0 | 0 | 0 | 90* |
| 0.0390625 | 0.0078125 | 0 | 0 | 0 | 70* |
| 0.15625 | 0.0625 | 0 | 70 | 70 | 100* |
| 0.078125 | 0.03125 | 0 | 20 | 20 | 100* |
| 0.0390625 | 0.015625 | 0 | 0 | 0 | 90* |

TABLE 162

| | | AVERAGE | | | |
|---|---|---|---|---|---|
| PPM AI | | DEAD IN % | | | |
| | Chloro- | | Chloro- | EXPECTED | OBSERVED |
| A1 | thalonil | A1 | thalonil | CONTROL | CONTROL |
| 10 | 0.25 | 0 | 50 | 50 | 50 |
| 5 | 0.125 | 0 | 20 | 20 | 20 |
| 5 | 0.25 | 0 | 50 | 50 | 50 |
| 2.5 | 0.125 | 0 | 20 | 20 | 20 |
| 2.5 | 0.25 | 0 | 50 | 50 | 50 |
| 1.25 | 0.125 | 0 | 20 | 20 | 20 |
| 1.25 | 0.25 | 0 | 50 | 50 | 50 |
| 0.625 | 0.125 | 0 | 20 | 20 | 20 |
| 0.625 | 0.25 | 0 | 50 | 50 | 50 |
| 0.3125 | 0.125 | 0 | 20 | 20 | 20 |

TABLE 163

| | | AVERAGE | | | |
|---|---|---|---|---|---|
| PPM AI | | DEAD IN % | | | |
| | Cypro- | | Cypro- | EXPECTED | OBSERVED |
| A1 | conazole | A1 | conazole | CONTROL | CONTROL |
| 5 | 0.125 | 0 | 70 | 70 | 70 |
| 2.5 | 0.125 | 0 | 70 | 70 | 50 |
| 1.25 | 0.125 | 0 | 70 | 70 | 50 |
| 0.625 | 0.125 | 0 | 70 | 70 | 50 |
| 0.3125 | 0.125 | 0 | 70 | 70 | 50 |

TABLE 164

| | | AVERAGE | | | |
|---|---|---|---|---|---|
| PPM AI | | DEAD IN % | | | |
| | Difeno- | | Difeno- | EXPECTED | OBSERVED |
| A1 | conazole | A1 | conazole | CONTROL | CONTROL |
| 0.3125 | 0.0078125 | 0 | 50 | 50 | 50 |
| 0.15625 | 0.0078125 | 0 | 50 | 50 | 50 |
| 0.078125 | 0.0078125 | 0 | 50 | 50 | 100* |
| 0.0390625 | 0.00390625 | 0 | 0 | 0 | 70* |
| 0.0390625 | 0.0078125 | 0 | 50 | 50 | 100* |

TABLE 165

| | | AVERAGE | | | |
|---|---|---|---|---|---|
| PPM AI | | DEAD IN % | | | |
| | COM- | | COM- | EXPECTED | OBSERVED |
| A1 | POUND III | A1 | POUND III | CONTROL | CONTROL |
| 2.5 | 0.0625 | 0 | 90 | 90 | 90 |
| 1.25 | 0.03125 | 0 | 20 | 20 | 20 |
| 0.625 | 0.015625 | 0 | 0 | 0 | 20* |
| 1.25 | 0.0625 | 0 | 90 | 90 | 90 |
| 0.625 | 0.03125 | 0 | 20 | 20 | 20 |
| 0.3125 | 0.015625 | 0 | 0 | 0 | 20* |
| 0.625 | 0.0625 | 0 | 90 | 90 | 90 |
| 0.3125 | 0.03125 | 0 | 20 | 20 | 70* |
| 0.15625 | 0.015625 | 0 | 0 | 0 | 20* |
| 0.3125 | 0.0625 | 0 | 90 | 90 | 100* |
| 0.15625 | 0.03125 | 0 | 20 | 20 | 100* |
| 0.078125 | 0.015625 | 0 | 0 | 0 | 100* |
| 0.0390625 | 0.0078125 | 0 | 0 | 0 | 90* |
| 0.15625 | 0.0625 | 0 | 90 | 90 | 100* |
| 0.078125 | 0.03125 | 0 | 20 | 20 | 100* |
| 0.0390625 | 0.015625 | 0 | 0 | 0 | 100* |

TABLE 166

| | | AVERAGE | | | |
|---|---|---|---|---|---|
| PPM AI | | DEAD IN % | | | |
| | COM- | | COM- | EXPECTED | OBSERVED |
| A1 | POUND IV | A1 | POUND IV | CONTROL | CONTROL |
| 5 | 0.125 | 0 | 90 | 90 | 70 |
| 2.5 | 0.0625 | 0 | 20 | 20 | 20 |
| 2.5 | 0.125 | 0 | 90 | 90 | 70 |
| 1.25 | 0.0625 | 0 | 20 | 20 | 20 |
| 1.25 | 0.125 | 0 | 90 | 90 | 70 |
| 0.625 | 0.0625 | 0 | 20 | 20 | 20 |
| 0.625 | 0.125 | 0 | 90 | 90 | 70 |
| 0.3125 | 0.125 | 0 | 90 | 90 | 90 |
| 0.15625 | 0.0625 | 0 | 20 | 20 | 20 |

TABLE 167

| | | AVERAGE | | | |
|---|---|---|---|---|---|
| PPM AI | | DEAD IN % | | | |
| | Azoxy- | | Azoxy- | EXPECTED | OBSERVED |
| A5 | strobin | A5 | strobin | CONTROL | CONTROL |
| 5 | 0.125 | 0 | 90 | 90 | 90 |
| 2.5 | 0.0625 | 0 | 50 | 50 | 70* |
| 1.25 | 0.03125 | 0 | 20 | 20 | 20 |
| 2.5 | 0.125 | 0 | 90 | 90 | 90 |
| 1.25 | 0.0625 | 0 | 50 | 50 | 70* |
| 0.625 | 0.03125 | 0 | 20 | 20 | 20 |
| 1.25 | 0.125 | 0 | 90 | 90 | 90 |
| 0.625 | 0.0625 | 0 | 50 | 50 | 70* |
| 0.3125 | 0.03125 | 0 | 20 | 20 | 20 |
| 0.625 | 0.125 | 0 | 90 | 90 | 90 |
| 0.3125 | 0.0625 | 0 | 50 | 50 | 70* |
| 0.15625 | 0.03125 | 0 | 20 | 20 | 20 |
| 0.3125 | 0.125 | 0 | 90 | 90 | 100* |
| 0.15625 | 0.0625 | 0 | 50 | 50 | 70* |
| 0.078125 | 0.03125 | 0 | 20 | 20 | 20 |

TABLE 168

| | | AVERAGE | | | |
|---|---|---|---|---|---|
| PPM AI | | DEAD IN % | | EXPECTED | OBSERVED |
| A5 | Isopyrazam | A5 | Isopyrazam | CONTROL | CONTROL |
| 2.5 | 0.0625 | 0 | 90 | 90 | 90 |
| 1.25 | 0.03125 | 0 | 20 | 20 | 20 |
| 1.25 | 0.0625 | 0 | 90 | 90 | 70 |
| 0.625 | 0.03125 | 0 | 20 | 20 | 20 |
| 0.625 | 0.0625 | 0 | 90 | 90 | 70 |
| 0.3125 | 0.03125 | 0 | 20 | 20 | 20 |
| 0.3125 | 0.0625 | 0 | 90 | 90 | 90 |
| 0.15625 | 0.03125 | 0 | 20 | 20 | 20 |
| 0.15625 | 0.0625 | 0 | 90 | 90 | 90 |
| 0.078125 | 0.03125 | 0 | 20 | 20 | 20 |

TABLE 169

| PPM AI | | AVERAGE | | | |
|---|---|---|---|---|---|
| | Chlo- | DEAD IN % | | EXPECTED | OBSERVED |
| A5 | rothalonil | A5 | Chlorothalonil | CONTROL | CONTROL |
| 20 | 0.5 | 0 | 70 | 70 | 100* |
| 10 | 0.5 | 0 | 70 | 70 | 100* |
| 5 | 0.5 | 0 | 70 | 70 | 100* |
| 2.5 | 0.5 | 0 | 70 | 70 | 100* |
| 1.25 | 0.25 | 0 | 0 | 0 | 20* |
| 1.25 | 0.5 | 0 | 70 | 70 | 100* |
| 0.625 | 0.25 | 0 | 0 | 0 | 20* |

TABLE 170

| PPM AI | | AVERAGE | | | |
|---|---|---|---|---|---|
| | Cypro- | DEAD IN % | | EXPECTED | OBSERVED |
| A5 | conazole | A5 | Cyproconazole | CONTROL | CONTROL |
| 10 | 0.25 | 0 | 70 | 70 | 70 |
| 5 | 0.125 | 0 | 20 | 20 | 20 |
| 5 | 0.25 | 0 | 70 | 70 | 70 |
| 2.5 | 0.125 | 0 | 20 | 20 | 20 |
| 2.5 | 0.25 | 0 | 70 | 70 | 70 |
| 1.25 | 0.125 | 0 | 20 | 20 | 20 |
| 1.25 | 0.25 | 0 | 70 | 70 | 70 |
| 0.625 | 0.125 | 0 | 20 | 20 | 0 |
| 0.625 | 0.25 | 0 | 70 | 70 | 70 |
| 0.3125 | 0.125 | 0 | 20 | 20 | 20 |
| 0.15625 | 0.0625 | 0 | 0 | 0 | 20* |

TABLE 171

| PPM AI | | AVERAGE | | | |
|---|---|---|---|---|---|
| | Difeno- | DEAD IN % | | EXPECTED | OBSERVED |
| A5 | conazole | A5 | Difenoconazole | CONTROL | CONTROL |
| 0.625 | 0.015625 | 0 | 50 | 50 | 50 |
| 0.3125 | 0.015625 | 0 | 50 | 50 | 50 |
| 0.15625 | 0.0078125 | 0 | 0 | 0 | 20* |
| 0.15625 | 0.015625 | 0 | 50 | 50 | 50 |
| 0.078125 | 0.015625 | 0 | 50 | 50 | 90* |
| 0.0390625 | 0.015625 | 0 | 50 | 50 | 50 |

TABLE 172

| | | AVERAGE | | | |
|---|---|---|---|---|---|
| PPM AI | | DEAD IN % | | | |
| | COM- | | COM- | EXPECTED | OBSERVED |
| A5 | POUND III | A5 | POUND III | CONTROL | CONTROL |
| 1.25 | 0.03125 | 0 | 70 | 70 | 70 |
| 0.625 | 0.03125 | 0 | 70 | 70 | 70 |
| 0.3125 | 0.03125 | 0 | 70 | 70 | 70 |
| 0.15625 | 0.03125 | 0 | 70 | 70 | 90* |
| 0.078125 | 0.015625 | 0 | 0 | 0 | 20* |
| 0.15625 | 0.0625 | 0 | 100 | 100 | 90 |
| 0.078125 | 0.03125 | 0 | 70 | 70 | 70 |
| 0.0390625 | 0.015625 | 0 | 0 | 0 | 20* |

TABLE 173

| | | AVERAGE | | | |
|---|---|---|---|---|---|
| PPM AI | | DEAD IN % | | | |
| | COM- | | COMPOUND | EXPECTED | OBSERVED |
| A5 | POUND IV | A5 | IV | CONTROL | CONTROL |
| 5 | 0.125 | 0 | 90 | 90 | 90 |
| 2.5 | 0.0625 | 0 | 50 | 50 | 50 |
| 2.5 | 0.125 | 0 | 90 | 90 | 90 |
| 1.25 | 0.0625 | 0 | 50 | 50 | 50 |
| 1.25 | 0.125 | 0 | 90 | 90 | 90 |
| 0.625 | 0.0625 | 0 | 50 | 50 | 50 |
| 0.625 | 0.125 | 0 | 90 | 90 | 90 |
| 0.3125 | 0.0625 | 0 | 50 | 50 | 50 |
| 0.3125 | 0.125 | 0 | 90 | 90 | 90 |
| 0.15625 | 0.0625 | 0 | 50 | 50 | 20 |

TABLE 174

| PPM AI | | AVERAGE | | | |
|---|---|---|---|---|---|
| | Azoxy- | DEAD IN % | | EXPECTED | OBSERVED |
| A6 | strobin | A6 | Azoxystrobin | CONTROL | CONTROL |
| 2.5 | 0.0625 | 0 | 90 | 90 | 90 |
| 1.25 | 0.03125 | 0 | 50 | 50 | 50 |
| 0.625 | 0.015625 | 0 | 20 | 20 | 20 |
| 1.25 | 0.0625 | 0 | 90 | 90 | 90 |
| 0.625 | 0.03125 | 0 | 50 | 50 | 50 |
| 0.3125 | 0.015625 | 0 | 20 | 20 | 20 |
| 0.625 | 0.0625 | 0 | 90 | 90 | 70 |
| 0.3125 | 0.03125 | 0 | 50 | 50 | 50 |
| 0.15625 | 0.015625 | 0 | 20 | 20 | 20 |
| 0.3125 | 0.0625 | 0 | 90 | 90 | 90 |
| 0.15625 | 0.03125 | 0 | 50 | 50 | 50 |
| 0.078125 | 0.015625 | 0 | 20 | 20 | 20 |
| 0.0390625 | 0.0078125 | 0 | 0 | 0 | 0 |
| 0.15625 | 0.0625 | 0 | 90 | 90 | 90 |
| 0.078125 | 0.03125 | 0 | 50 | 50 | 50 |
| 0.0390625 | 0.015625 | 0 | 20 | 20 | 20 |

TABLE 175

| | | AVERAGE | | | |
|---|---|---|---|---|---|
| PPM AI | | DEAD IN % | | EXPECTED | OBSERVED |
| A6 | Isopyrazam | A6 | Isopyrazam | CONTROL | CONTROL |
| 2.5 | 0.0625 | 0 | 50 | 50 | 70* |
| 1.25 | 0.0625 | 0 | 50 | 50 | 70* |
| 0.625 | 0.03125 | 0 | 20 | 20 | 20 |
| 0.625 | 0.0625 | 0 | 50 | 50 | 70* |
| 0.3125 | 0.03125 | 0 | 20 | 20 | 20 |
| 0.3125 | 0.0625 | 0 | 50 | 50 | 70* |
| 0.15625 | 0.03125 | 0 | 20 | 20 | 20 |
| 0.15625 | 0.0625 | 0 | 50 | 50 | 50 |
| 0.078125 | 0.03125 | 0 | 20 | 20 | 20 |

TABLE 176

| PPM AI | | AVERAGE | | | |
|---|---|---|---|---|---|
| | Chloro- | DEAD IN % | | EXPECTED | OBSERVED |
| A6 | thalonil | A6 | Chlorothalonil | CONTROL | CONTROL |
| 20 | 0.5 | 0 | 100 | 100 | 100 |
| 10 | 0.25 | 0 | 20 | 20 | 20 |
| 5 | 0.25 | 0 | 20 | 20 | 20 |
| 2.5 | 0.25 | 0 | 20 | 20 | 20 |
| 1.25 | 0.25 | 0 | 20 | 20 | 20 |
| 0.625 | 0.25 | 0 | 20 | 20 | 20 |

TABLE 177

| PPM AI | | AVERAGE | | | |
|---|---|---|---|---|---|
| | Cypro- | DEAD IN % | | EXPECTED | OBSERVED |
| A6 | conazole | A6 | Cyproconazole | CONTROL | CONTROL |
| 5 | 0.125 | 0 | 20 | 20 | 20 |
| 2.5 | 0.125 | 0 | 20 | 20 | 20 |
| 1.25 | 0.125 | 0 | 20 | 20 | 20 |
| 1.25 | 0.25 | 0 | 100 | 100 | 90 |
| 0.625 | 0.125 | 0 | 20 | 20 | 20 |
| 0.625 | 0.25 | 0 | 100 | 100 | 90 |
| 0.3125 | 0.125 | 0 | 20 | 20 | 20 |

TABLE 178

| | | AVERAGE | | | |
|---|---|---|---|---|---|
| | | DEAD IN % | | | |
| PPM AI | | | Difeno- | EXPECTED | OBSERVED |
| A6 | Difenoconazole | A6 | conazole | CONTROL | CONTROL |
| 0.625 | 0.015625 | 0 | 90 | 90 | 70 |
| 0.3125 | 0.0078125 | 0 | 20 | 20 | 20 |
| 0.15625 | 0.00390625 | 0 | 0 | 0 | 0 |
| 0.3125 | 0.015625 | 0 | 90 | 90 | 50 |
| 0.15625 | 0.0078125 | 0 | 20 | 20 | 20 |
| 0.15625 | 0.015625 | 0 | 90 | 90 | 100* |
| 0.078125 | 0.0078125 | 0 | 20 | 20 | 20 |
| 0.078125 | 0.015625 | 0 | 90 | 90 | 90 |
| 0.0390625 | 0.0078125 | 0 | 20 | 20 | 20 |
| 0.0390625 | 0.015625 | 0 | 90 | 90 | 70 |

TABLE 179

| | | AVERAGE | | | |
|---|---|---|---|---|---|
| PPM AI | | DEAD IN % | | | |
| | COM- | | COM- | EXPECTED | OBSERVED |
| A6 | POUND III | A6 | POUND III | CONTROL | CONTROL |
| 5 | 0.125 | 0 | 90 | 90 | 90 |
| 2.5 | 0.0625 | 0 | 70 | 70 | 70 |
| 1.25 | 0.03125 | 0 | 50 | 50 | 20 |
| 0.625 | 0.015625 | 0 | 20 | 20 | 20 |
| 2.5 | 0.125 | 0 | 90 | 90 | 90 |
| 1.25 | 0.0625 | 0 | 70 | 70 | 70 |
| 0.625 | 0.03125 | 0 | 50 | 50 | 20 |
| 0.3125 | 0.015625 | 0 | 20 | 20 | 20 |
| 1.25 | 0.125 | 0 | 90 | 90 | 100* |
| 0.625 | 0.0625 | 0 | 70 | 70 | 90* |
| 0.3125 | 0.03125 | 0 | 50 | 50 | 50 |
| 0.15625 | 0.015625 | 0 | 20 | 20 | 20 |
| 0.625 | 0.125 | 0 | 90 | 90 | 100* |
| 0.3125 | 0.0625 | 0 | 70 | 70 | 70 |
| 0.15625 | 0.03125 | 0 | 50 | 50 | 20 |
| 0.078125 | 0.015625 | 0 | 20 | 20 | 0 |
| 0.3125 | 0.125 | 0 | 90 | 90 | 100* |
| 0.15625 | 0.0625 | 0 | 70 | 70 | 70 |
| 0.078125 | 0.03125 | 0 | 50 | 50 | 20 |
| 0.0390625 | 0.015625 | 0 | 20 | 20 | 20 |

TABLE 180

| | | AVERAGE | | | |
|---|---|---|---|---|---|
| PPM AI | | DEAD IN % | | | |
| | COM- | | COMPOUND | EXPECTED | OBSERVED |
| A6 | POUND IV | A6 | IV | CONTROL | CONTROL |
| 5 | 0.125 | 0 | 90 | 90 | 90 |
| 2.5 | 0.0625 | 0 | 20 | 20 | 20 |
| 2.5 | 0.125 | 0 | 90 | 90 | 90 |
| 1.25 | 0.0625 | 0 | 20 | 20 | 20 |
| 1.25 | 0.125 | 0 | 90 | 90 | 90 |
| 0.625 | 0.0625 | 0 | 20 | 20 | 20 |
| 0.625 | 0.125 | 0 | 90 | 90 | 90 |
| 0.3125 | 0.0625 | 0 | 20 | 20 | 50* |
| 0.3125 | 0.125 | 0 | 90 | 90 | 90 |
| 0.15625 | 0.0625 | 0 | 20 | 20 | 20 |

TABLE 181

| PPM AI | | AVERAGE | | | |
|---|---|---|---|---|---|
| | Azoxy- | DEAD IN % | | EXPECTED | OBSERVED |
| A8 | strobin | A8 | Azoxystrobin | CONTROL | CONTROL |
| 2.5 | 0.0625 | 0 | 90 | 90 | 90 |
| 1.25 | 0.03125 | 0 | 70 | 70 | 70 |
| 0.625 | 0.015625 | 0 | 20 | 20 | 20 |
| 1.25 | 0.0625 | 0 | 90 | 90 | 90 |
| 0.625 | 0.03125 | 0 | 70 | 70 | 70 |
| 0.3125 | 0.015625 | 0 | 20 | 20 | 20 |
| 0.625 | 0.0625 | 0 | 90 | 90 | 90 |
| 0.3125 | 0.03125 | 0 | 70 | 70 | 50 |
| 0.15625 | 0.015625 | 0 | 20 | 20 | 20 |
| 0.3125 | 0.0625 | 0 | 90 | 90 | 90 |
| 0.15625 | 0.03125 | 0 | 70 | 70 | 50 |
| 0.078125 | 0.015625 | 0 | 20 | 20 | 20 |
| 0.15625 | 0.0625 | 0 | 90 | 90 | 90 |
| 0.078125 | 0.03125 | 0 | 70 | 70 | 50 |
| 0.0390625 | 0.015625 | 0 | 20 | 20 | 20 |

TABLE 182

| | | AVERAGE | | | |
|---|---|---|---|---|---|
| PPM AI | | DEAD IN % | | EXPECTED | OBSERVED |
| A8 | Isopyrazam | A8 | Isopyrazam | CONTROL | CONTROL |
| 2.5 | 0.0625 | 0 | 70 | 70 | 70 |
| 1.25 | 0.03125 | 0 | 20 | 20 | 20 |
| 1.25 | 0.0625 | 0 | 70 | 70 | 70 |
| 0.625 | 0.03125 | 0 | 20 | 20 | 20 |
| 0.625 | 0.0625 | 0 | 70 | 70 | 70 |
| 0.3125 | 0.03125 | 0 | 20 | 20 | 20 |
| 0.3125 | 0.0625 | 0 | 70 | 70 | 90* |
| 0.15625 | 0.03125 | 0 | 20 | 20 | 20 |
| 0.078125 | 0.015625 | 0 | 0 | 0 | 20* |
| 0.15625 | 0.0625 | 0 | 70 | 70 | 70 |
| 0.078125 | 0.03125 | 0 | 20 | 20 | 20 |

TABLE 183

| PPM AI | | AVERAGE | | | |
|---|---|---|---|---|---|
| | Chloro- | DEAD IN % | | EXPECTED | OBSERVED |
| A8 | thalonil | A8 | Chlorothalonil | CONTROL | CONTROL |
| 20 | 0.5 | 0 | 70 | 70 | 90* |
| 10 | 0.25 | 0 | 20 | 20 | 20 |
| 10 | 0.5 | 0 | 70 | 70 | 90* |
| 5 | 0.25 | 0 | 20 | 20 | 20 |
| 5 | 0.5 | 0 | 70 | 70 | 100* |
| 2.5 | 0.25 | 0 | 20 | 20 | 20 |
| 2.5 | 0.5 | 0 | 70 | 70 | 100* |
| 1.25 | 0.25 | 0 | 20 | 20 | 20 |
| 1.25 | 0.5 | 0 | 70 | 70 | 70 |
| 0.625 | 0.25 | 0 | 20 | 20 | 20 |

TABLE 184

| | | | | | OB- |
|---|---|---|---|---|---|
| | | AVERAGE | | | SERVED |
| PPM AI | | DEAD IN % | | EXPECTED | CON- |
| A8 | Cyproconazole | A8 | Cyproconazole | CONTROL | TROL |
| 20 | 0.5 | 0 | 100 | 100 | 100 |
| 10 | 0.25 | 0 | 100 | 100 | 100 |
| 5 | 0.125 | 0 | 20 | 20 | 20 |
| 2.5 | 0.125 | 0 | 20 | 20 | 20 |
| 1.25 | 0.125 | 0 | 20 | 20 | 20 |
| 0.625 | 0.125 | 0 | 20 | 20 | 20 |
| 0.3125 | 0.125 | 0 | 20 | 20 | 20 |

TABLE 185

| | | | | EX- | OB- |
|---|---|---|---|---|---|
| | | AVERAGE | | PECTED | SERVED |
| PPM AI | | DEAD IN % | | CON- | CON- |
| A8 | Difenoconazole | A8 | Difenoconazole | TROL | TROL |
| 0.625 | 0.015625 | 0 | 90 | 90 | 90 |
| 0.3125 | 0.0078125 | 0 | 20 | 20 | 20 |
| 0.3125 | 0.015625 | 0 | 90 | 90 | 100* |
| 0.15625 | 0.0078125 | 0 | 20 | 20 | 20 |
| 0.15625 | 0.015625 | 0 | 90 | 90 | 70 |
| 0.078125 | 0.0078125 | 0 | 20 | 20 | 20 |
| 0.0390625 | 0.00390625 | 0 | 0 | 0 | 0 |
| 0.078125 | 0.015625 | 0 | 90 | 90 | 90 |
| 0.0390625 | 0.0078125 | 0 | 20 | 20 | 20 |
| 0.0390625 | 0.015625 | 0 | 90 | 90 | 100* |

TABLE 186

| | | AVERAGE | | | |
|---|---|---|---|---|---|
| PPM AI | | DEAD IN % | | | |
| | COM- | | COM- | EXPECTED | OBSERVED |
| A8 | POUND III | A8 | POUND III | CONTROL | CONTROL |
| 2.5 | 0.0625 | 0 | 90 | 90 | 70 |
| 1.25 | 0.03125 | 0 | 50 | 50 | 20 |
| 0.625 | 0.015625 | 0 | 20 | 20 | 20 |
| 1.25 | 0.0625 | 0 | 90 | 90 | 70 |
| 0.625 | 0.03125 | 0 | 50 | 50 | 20 |
| 0.3125 | 0.015625 | 0 | 20 | 20 | 20 |
| 0.625 | 0.0625 | 0 | 90 | 90 | 70 |
| 0.3125 | 0.03125 | 0 | 50 | 50 | 20 |
| 0.15625 | 0.015625 | 0 | 20 | 20 | 20 |
| 0.3125 | 0.0625 | 0 | 90 | 90 | 90 |
| 0.15625 | 0.03125 | 0 | 50 | 50 | 50 |
| 0.078125 | 0.015625 | 0 | 20 | 20 | 20 |
| 0.15625 | 0.0625 | 0 | 90 | 90 | 70 |
| 0.078125 | 0.03125 | 0 | 50 | 50 | 50 |
| 0.0390625 | 0.015625 | 0 | 20 | 20 | 50* |

TABLE 187

| | | AVERAGE | | | |
|---|---|---|---|---|---|
| PPM AI | | DEAD IN % | | | |
| | COM- | | COMPOUND | EXPECTED | OBSERVED |
| A8 | POUND IV | A8 | IV | CONTROL | CONTROL |
| 5 | 0.125 | 0 | 90 | 90 | 90 |
| 2.5 | 0.0625 | 0 | 20 | 20 | 20 |
| 2.5 | 0.125 | 0 | 90 | 90 | 90 |
| 1.25 | 0.0625 | 0 | 20 | 20 | 20 |
| 1.25 | 0.125 | 0 | 90 | 90 | 90 |
| 0.625 | 0.0625 | 0 | 20 | 20 | 20 |
| 0.625 | 0.125 | 0 | 90 | 90 | 90 |
| 0.3125 | 0.0625 | 0 | 20 | 20 | 20 |
| 0.3125 | 0.125 | 0 | 90 | 90 | 70 |
| 0.15625 | 0.0625 | 0 | 20 | 20 | 20 |

Fusarium culmorum (Root Rot):

Conidia of the fungus from cryogenic storage were directly mixed into nutrient broth (potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 48 hrs. Application rates are shown in the Tables.

TABLE 188

| | | AVERAGE | | | |
|---|---|---|---|---|---|
| PPM AI | | DEAD IN % | | EXPECTED | OBSERVED |
| A1 | Chlorothalonil | A1 | Chlorothalonil | CONTROL | CONTROL |
| 20 | 1 | 0 | 20 | 20 | 50* |
| 10 | 1 | 0 | 20 | 20 | 50* |
| 5 | 1 | 0 | 20 | 20 | 50* |
| 2.5 | 0.5 | 0 | 0 | 0 | 20* |
| 2.5 | 1 | 0 | 20 | 20 | 50* |
| 1.25 | 0.5 | 0 | 0 | 0 | 20* |

TABLE 189

| | | AVERAGE | | | |
|---|---|---|---|---|---|
| PPM AI | | DEAD IN % | | EXPECTED | OBSERVED |
| A1 | Cyproconazole | A1 | Cyproconazole | CONTROL | CONTROL |
| 20 | 2 | 0 | 20 | 20 | 20 |
| 10 | 2 | 0 | 20 | 20 | 20 |
| 5 | 2 | 0 | 20 | 20 | 20 |

TABLE 190

| | | | | | OB- |
|---|---|---|---|---|---|
| | | AVERAGE | | | SERVED |
| PPM AI | | DEAD IN % | | EXPECTED | CON- |
| A1 | Difenoconazole | A1 | Difenoconazole | CONTROL | TROL |
| 20 | 0.5 | 0 | 20 | 20 | 0 |
| 10 | 0.25 | 0 | 20 | 20 | 0 |
| 20 | 1 | 0 | 20 | 20 | 20 |
| 10 | 0.5 | 0 | 20 | 20 | 0 |
| 5 | 0.25 | 0 | 20 | 20 | 0 |
| 20 | 2 | 0 | 90 | 90 | 20 |
| 10 | 1 | 0 | 20 | 20 | 20 |
| 5 | 0.5 | 0 | 20 | 20 | 0 |
| 2.5 | 0.25 | 0 | 20 | 20 | 0 |
| 10 | 2 | 0 | 90 | 90 | 50 |
| 5 | 1 | 0 | 20 | 20 | 20 |
| 2.5 | 0.5 | 0 | 20 | 20 | 0 |
| 1.25 | 0.25 | 0 | 20 | 20 | 0 |
| 5 | 2 | 0 | 90 | 90 | 70 |
| 2.5 | 1 | 0 | 20 | 20 | 20 |
| 1.25 | 0.5 | 0 | 20 | 20 | 20 |
| 0.625 | 0.25 | 0 | 20 | 20 | 0 |

TABLE 191

| | | AVERAGE | | | |
|---|---|---|---|---|---|
| PPM AI | | DEAD IN % | | | |
| | COM- | | COMPOUND | EXPECTED | OBSERVED |
| A1 | POUND IV | A1 | IV | CONTROL | CONTROL |
| 20 | 1 | 0 | 20 | 20 | 20 |
| 20 | 2 | 0 | 50 | 50 | 50 |
| 10 | 1 | 0 | 20 | 20 | 20 |
| 10 | 2 | 0 | 50 | 50 | 50 |
| 5 | 1 | 0 | 20 | 20 | 20 |
| 5 | 2 | 0 | 50 | 50 | 50 |
| 2.5 | 1 | 0 | 20 | 20 | 20 |

TABLE 192

| | | AVERAGE | | | |
|---|---|---|---|---|---|
| PPM AI | | DEAD IN % | | EXPECTED | OBSERVED |
| A5 | Azoxystrobin | A5 | Azoxystrobin | CONTROL | CONTROL |
| 20 | 0.5 | 0 | 0 | 0 | 20* |
| 20 | 1 | 0 | 20 | 20 | 20 |
| 10 | 0.5 | 0 | 0 | 0 | 20* |
| 20 | 2 | 0 | 20 | 20 | 20 |
| 10 | 1 | 0 | 20 | 20 | 20 |
| 5 | 0.5 | 0 | 0 | 0 | 20* |
| 10 | 2 | 0 | 20 | 20 | 20 |
| 5 | 1 | 0 | 20 | 20 | 20 |
| 2.5 | 0.5 | 0 | 0 | 0 | 20* |
| 5 | 2 | 0 | 20 | 20 | 20 |
| 2.5 | 1 | 0 | 20 | 20 | 20 |
| 1.25 | 0.5 | 0 | 0 | 0 | 20* |

TABLE 193

| | | AVERAGE | | | |
|---|---|---|---|---|---|
| PPM AI | | DEAD IN % | | EXPECTED | OBSERVED |
| A5 | Chlorothalonil | A5 | Chlorothalonil | CONTROL | CONTROL |
| 20 | 0.5 | 0 | 0 | 0 | 20* |
| 20 | 1 | 0 | 50 | 50 | 90* |
| 10 | 0.5 | 0 | 0 | 0 | 20* |
| 10 | 1 | 0 | 50 | 50 | 70* |
| 5 | 0.5 | 0 | 0 | 0 | 20* |
| 5 | 1 | 0 | 50 | 50 | 100* |
| 2.5 | 0.5 | 0 | 0 | 0 | 20* |
| 2.5 | 1 | 0 | 50 | 50 | 90* |
| 1.25 | 0.5 | 0 | 0 | 0 | 20* |

TABLE 194

| | | AVERAGE | | | |
|---|---|---|---|---|---|
| PPM AI | | DEAD IN % | | EXPECTED | OBSERVED |
| A5 | Cyproconazole | A5 | Cyproconazole | CONTROL | CONTROL |
| 20 | 2 | 0 | 20 | 20 | 20 |
| 10 | 2 | 0 | 20 | 20 | 20 |
| 5 | 2 | 0 | 20 | 20 | 20 |

TABLE 195

| | | | | | OB- |
|---|---|---|---|---|---|
| | | AVERAGE | | | SERVED |
| PPM AI | | DEAD IN % | | EXPECTED | CON- |
| A5 | Difenoconazole | A5 | Difenoconazole | CONTROL | TROL |
| 20 | 0.5 | 0 | 20 | 20 | 20 |
| 10 | 0.25 | 0 | 20 | 20 | 20 |
| 20 | 1 | 0 | 70 | 70 | 50 |
| 10 | 0.5 | 0 | 20 | 20 | 20 |
| 5 | 0.25 | 0 | 20 | 20 | 20 |
| 10 | 1 | 0 | 70 | 70 | 50 |
| 5 | 0.5 | 0 | 20 | 20 | 20 |
| 2.5 | 0.25 | 0 | 20 | 20 | 20 |
| 5 | 1 | 0 | 70 | 70 | 70 |
| 2.5 | 0.5 | 0 | 20 | 20 | 20 |
| 1.25 | 0.25 | 0 | 20 | 20 | 20 |
| 2.5 | 1 | 0 | 70 | 70 | 70 |
| 1.25 | 0.5 | 0 | 20 | 20 | 20 |
| 0.625 | 0.25 | 0 | 20 | 20 | 20 |

TABLE 196

| | | AVERAGE | | | |
|---|---|---|---|---|---|
| PPM AI | | DEAD IN % | | | |
| | COMPOUND | | COMPOUND | EXPECTED | OBSERVED |
| A5 | IV | A5 | IV | CONTROL | CONTROL |
| 20 | 1 | 0 | 20 | 20 | 20 |
| 20 | 2 | 0 | 50 | 50 | 50 |
| 10 | 1 | 0 | 20 | 20 | 20 |
| 10 | 2 | 0 | 50 | 50 | 50 |
| 5 | 1 | 0 | 20 | 20 | 20 |
| 5 | 2 | 0 | 50 | 50 | 50 |
| 2.5 | 1 | 0 | 20 | 20 | 20 |

TABLE 197

| | | AVERAGE | | | |
|---|---|---|---|---|---|
| PPM AI | | DEAD IN % | | | |
| | COMPOUND | | COMPOUND | EXPECTED | OBSERVED |
| A6 | IV | A6 | IV | CONTROL | CONTROL |
| 20 | 1 | 0 | 20 | 20 | 20 |
| 10 | 0.5 | 0 | 0 | 0 | 20* |
| 20 | 2 | 0 | 50 | 50 | 50 |
| 10 | 1 | 0 | 20 | 20 | 20 |
| 10 | 2 | 0 | 50 | 50 | 50 |
| 5 | 1 | 0 | 20 | 20 | 20 |
| 5 | 2 | 0 | 50 | 50 | 50 |
| 2.5 | 1 | 0 | 20 | 20 | 20 |

TABLE 198

| | | AVERAGE | | | |
|---|---|---|---|---|---|
| PPM AI | | DEAD IN % | | | |
| | COMPOUND | | COMPOUND | EXPECTED | OBSERVED |
| A8 | IV | A8 | IV | CONTROL | CONTROL |
| 20 | 1 | 0 | 20 | 20 | 20 |
| 10 | 0.5 | 0 | 0 | 0 | 20* |
| 20 | 2 | 0 | 50 | 50 | 50 |
| 10 | 1 | 0 | 20 | 20 | 20 |
| 10 | 2 | 0 | 50 | 50 | 50 |
| 5 | 1 | 0 | 20 | 20 | 20 |
| 5 | 2 | 0 | 50 | 50 | 50 |
| 2.5 | 1 | 0 | 20 | 20 | 20 |

Data is not shown for experiments where there was no fungal death or where one compound and the combination of compounds killed 100%.

1. A pesticidal mixture comprising a component A and a component B, wherein component A is a compound of formula I 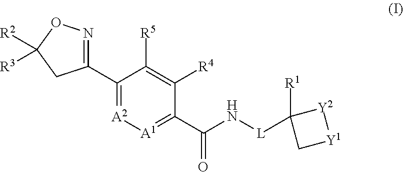
wherein
one of Y¹ and Y² is S, SO or SO₂ and the other is CH₂;
L is a direct bond or methylene;
A¹ and A² are C—H, or one of A¹ and A² is C—H and the other is N;
R¹ is hydrogen or methyl;
R² is chlorodifluoromethyl or trifluoromethyl;
R³ is 3,5-dibromo-phenyl, 3,5-dichloro-phenyl, 3,4-dichloro-phenyl, or 3,4,5-trichloro-phenyl;
R⁴ is methyl;
R⁵ is hydrogen;
or R⁴ and R⁵ together form a bridging 1,3-butadiene group;
and component B is a fungicide selected from
a strobilurin fungicide including those selected from the group consisting of: Azoxystrobin, Dimoxystrobin, Enestrobin, Fluoxastrobin, Kresoxim-methyl, Metominostrobin, Orysastrobin, Picoxystrobin, Pyraclostrobin and Trifloxystrobin;
an azole fungicide including those selected from the group consisting of: Azaconazole, Bromuconazole, Cyproconazole, Difenoconazole, Diniconazole, Diniconazole-M, Epoxiconazole, Fenbuconazole, Fluquinconazole, Flusilazole, Flutriafol, Hexaconazole, Imazalil, Imibenconazole, Ipconazole, Metconazole, Myclobutanil, Oxpoconazole, Pefurazoate, Penconazole, Prochloraz, Propiconazole, Prothioconazole, Simeconazole, Tebuconazole, Tetraconazole, Triadimefon, Triadimenol, Triflumizole, Triticonazole, Diclobutrazol, Etaconazole, Furconazole, Furconazole-cis, Thiabendazole and Quinconazole;
a phenyl pyrrole fungicide including those selected from the group consisting of: Fenpiclonil and Fludioxonil;
an anilino-pyrimidine fungicide including those selected from the group consisting of: Cyprodinil, Mepanipyrim and Pyrimethanil;
a morpholine fungicide including those selected from the group consisting of: Aldimorph, Dodemorph, Fenpropimorph, Tridemorph, Fenpropidin and Spiroxamine;
a carboxamide fungicide including those selected from the group consisting of: Isopyrazam, Sedaxane, Bixafen, Penthiopyrad, Fluxapyroxad, Boscalid, Penflufen, Fluopyram, a compound of formula II 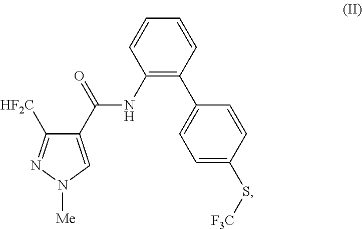
a compound of formula III 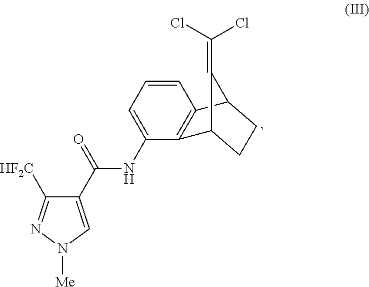
and a compound of formula IV 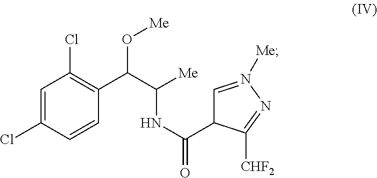
a carboxylic acid amide fungicide including those selected from the group consisting of: Mandipropamid, Benthiavalicarb and Dimethomorph;
Chlorothalonil, Fluazinam, Dithianon, Metrafenone, Tricyclazole, Mefenoxam, Metalaxyl, Acibenzolar, Mancozeb, Ametoctradine and Cyflufenamid.

2. A pesticidal mixture according to claim 1, wherein in the compound of formula I L is a direct bond or methylene; one of Y¹ and Y² is S and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.

3. A pesticidal mixture according to claim 1, wherein in the compound of formula I L is a direct bond or methylene; one of Y¹ and Y² is SO and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.

4. A pesticidal mixture according to claim 3, wherein the molar proportion of the cis SO compounds of formula I compared to the total amount of compounds of cis SO and trans SO compounds of formula I is greater than 50%.

5. A pesticidal mixture according to claim 1, wherein in the compound of formula I L is a direct bond or methylene; one of Y¹ and Y² is SO₂ and the other is CH₂; A¹ and A² are C—H; R¹ is hydrogen or methyl; R² is trifluoromethyl; R³ is 3,5-dichloro-phenyl; R⁴ is methyl; and R⁵ is hydrogen.

6. A pesticidal mixture according to claim 1, wherein when L is a direct bond Y² is CH₂ and Y¹ is S, SO or SO₂, and wherein when L is methylene Y² is S, SO or SO₂ and Y¹ is CH₂.

7. A pesticidal mixture according to claim 1, wherein component A is a mixture of compounds I* and I** 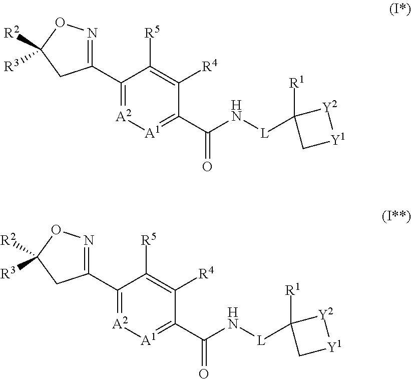
wherein the molar proportion of compound I** compared to the total amount of both enantiomers is greater than 50%.

8. A pesticidal mixture according to claim 1, wherein component B is a fungicide selected from
a strobilurin fungicide selected from the group consisting of: Azoxystrobin, Dimoxystrobin, Enestrobin, Fluoxastrobin, Kresoxim-methyl, Metominostrobin, Orysastrobin, Picoxystrobin, Pyraclostrobin and Trifloxystrobin;
an azole fungicide selected from the group consisting of: Azaconazole, Bromuconazole, Cyproconazole, Difenoconazole, Diniconazole, Diniconazole-M, Epoxiconazole, Fenbuconazole, Fluquinconazole, Flusilazole, Flutriafol, Hexaconazole, Imazalil, Imibenconazole, Ipconazole, Metconazole, Myclobutanil, Oxpoconazole, Pefurazoate, Penconazole, Prochloraz, Propiconazole, Prothioconazole, Simeconazole, Tebuconazole, Tetraconazole, Triadimefon, Triadimenol, Triflumizole, Triticonazole, Diclobutrazol, Etaconazole, Furconazole, Furconazole-cis, Thiabendazole and Quinconazole;
a phenyl pyrrole fungicide selected from the group consisting of: Fenpiclonil and Fludioxonil;
an anilino-pyrimidine fungicide selected from the group consisting of: Cyprodinil, Mepanipyrim and Pyrimethanil;
a morpholine fungicide selected from the group consisting of: Aldimorph, Dodemorph, Fenpropimorph, Tridemorph, Fenpropidin and Spiroxamine;
a carboxamide fungicide selected from the group consisting of: Isopyrazam, Sedaxane, Bixafen, Penthiopyrad, Fluxapyroxad, Boscalid, Penflufen, Fluopyram, a compound of formula II 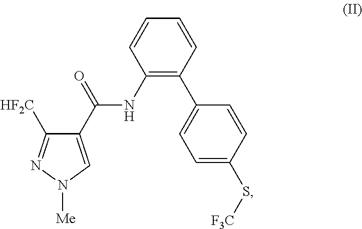
a compound of formula III 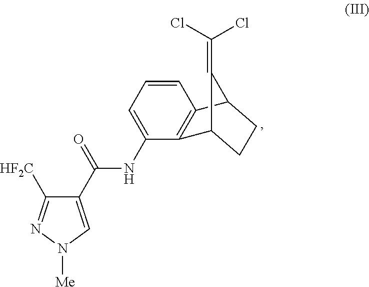
and a compound of formula IV 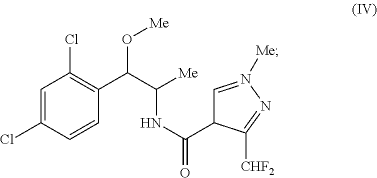
a carboxylic acid amide fungicide selected from the group consisting of: Mandipropamid, Benthiavalicarb and Dimethomorph;
Chlorothalonil, Fluazinam, Dithianon, Metrafenone, Tricyclazole, Mefenoxam, Metalaxyl, Acibenzolar, Mancozeb, Ametoctradine and Cyflufenamid.

9. A pesticidal mixture according to claim 1, wherein component B is a compound selected from the group consisting of Azoxystrobin, Isopyrazam, Chlorothalonil, Cyproconazole, Difenoconaozle, Mandipropamid, Mefenoxam, Metalaxyl, Sedaxane, Acibenzolar, Fludioxonil, Cyprodinil, Penconazole, Propiconazole, Mancozeb, Prothioconazole, Pyraclostrobin, Boscalid, Bixafen, Fluopyram, Penthiopyrad, Thiabendazole, Fluazinam, Fenpropidin, Cyclufenamid, Tebuconaozle, Trifoxystrobin, Fluxapyroxad, Penflufen, Fluoxastrobin, Kresoxim-methyl, Benthiavalicarb, Dimethomorph, a compound of formula II, a compound of formula III and a compound of formula IV.

10. A pesticidal mixture according to claim 1, wherein component B is a compound selected from the group consisting of Azoxystrobin, Isopyrazam, Chlorothalonil, Cyproconazole, Difenoconaozle, Mandipropamid, Mefanoxam, Metalaxyl, Sedaxane, Acibenzolar, Fludioxonil, Cyprodinil, Penconazole, Propiconazole, Mancozeb, Prothioconazole, Pyraclostrobin, Boscalid, Bixafen, Fluopyram, Penthiopyrad, Thiabendazole, a compound of formula III a compound of formula IV.

11. A pesticidal mixture according to claim 1, wherein component B is a compound selected from the group consisting of Azoxystrobin, Isopyrazam, Chlorothalonil, Cyproconazole, Difenoconaozle, Mandipropamid, Mefenoxam, Metalaxyl, Sedaxane, Acibenzolar, Fludioxonil, Thiabendazole, a compound of formula III and a compound of formula IV.

12. A pesticidal mixture according to claim 1, wherein component B is a compound selected from the group consisting of Azoxystrobin, Isopyrazam, Chlorothalonil, Cyproconazole, Difenoconaozle, Mandipropamid, Mefenoxam, a compound of formula III and a compound of formula IV.

13. A pesticidal mixture according to claim 1, wherein the mixture comprises an agricultural acceptable carrier and optionally a surfactant.

14. A pesticidal mixture according to claim 1, wherein the weight ratio of A to B is 1000:1 to 1:1000.

15. A method of controlling phytopathogenic diseases on useful plants or on propagation material thereof, which comprises applying to the useful plants, the locus thereof or propagation material thereof a combination of components a and b, wherein components a and b are as defined in claim 1.

16. A method of controlling insects, acarines, nematodes or molluscs which comprises applying to a pest, to a locus of a pest, or to a plant susceptible to attack by a pest a combination of components A and B, wherein components A and B are as defined in claim 1.

17. A seed comprising a pesticidal mixture as defined in claim 1.

18. A method comprising coating a seed with a mixture as defined in claim 1.
